(19) United States
(12) Patent Application Publication
(10) Pub. No.: US 2003/0013650 A1
(43) Pub. Date: Jan. 16, 2003

(54) VR-OAC, an osomotically activated channel protein, nucleic acids encoding it, and uses thereof

(75) Inventors: Wolfgang Liedtke, New York, NY (US); Stefan Heller, Rockland, MA (US); Albert James Hudspeth, New York, NY (US); Jeffrey M. Friedman, New York, NY (US)

Correspondence Address: KLAUBER & JACKSON, 411 HACKENSACK AVENUE, HACKENSACK, NJ, 07601

(73) Assignee: The Rockefeller University, New York, NY

(21) Appl. No.: 10/027,828

(22) Filed: Oct. 25, 2001

Related U.S. Application Data
(60) Provisional application No. 60/243,568, filed on Oct. 26, 2000.

(51) Int. Cl. A61K 38/17

(52) U.S. Cl. 514/12

(57) ABSTRACT

The present invention relates to the identification in vertebrate animals, including humans, of an ion channel which is involved in osmoregulation and mechanoreception. This ion channel, named VR-OAC, functions as a cation channel which is activated by osmotic and mechanical stimulation. In particular, the present invention relates to the broad applications of VR-OAC that capitalize on its newly discovered properties and activities, including both diagnostic and therapeutic methodologies. The invention further relates to methods for using the receptor therapeutically, such as polypeptide or gene therapy, diagnostically. and to methods and assays for identification and screening of VR-OAC analogs, agonists or antagonists and uses thereof.

The research leading to the present invention was supported, in part, by the following grants from the National Institutes of Health: GM54762, DC00317 and DK41096. Accordingly, the United States Government may have certain rights in the invention.

### FIELD OF THE INVENTION

The present invention relates to the identification in vertebrate animals including humans, of an ion channel for rapid conduction of cations, among them, Ca²⁺. This ion channel, named VR-OAC, demonstrates activity as an osmoreceptor, and also demonstrates a role in mechanical stimulation and responsiveness. In particular, the present invention relates to identification and characterization of a cellular receptor which functions as a cation channel which is activated by osmotic and mechanical stimulation, and more particularly to the broad applications of VR-OAC that capitalize on its newly discovered properties and activities, including both diagnostic and therapeutic methodologies.

### BACKGROUND OF THE INVENTION

Most organisms are sensitive to mechanical and osmotic stimulation (French, 1992). This responsiveness is thought to be mediated by proteins that measure the tension in membranes (Kernan and Zuker, 1995; Sackin, 1995) or other elastic elements (Hudspeth and Gillespie, 1994). The ability of higher organisms to sense and react to external and internal mechanical and osmotic stimuli depends on the electrical activity of sensory cells in response to touch, vibration, and osmotic pressure. In the inner ear, mechanosensory cells respond to sound and acceleration (Hudspeth, 1989). Exteroceptive and interoceptive nerve endings in skin and mucous membranes emanate from sensory ganglionic neurons and mediate responses to touch, vibration, and mechanical pain (Lynn, 1975; Gardner et al., 2000). In the vertebrate central nervous system, neurons in the circumventricular organs, areas of the brain without a blood-brain barrier, respond to changes in plasma osmolality and elicit adaptive changes in fluid and electrolyte intake and excretion (McKinley and Oldfield, 1990; Denton et al., 1996; Bourque and Oliet, 1997). The molecular mechanisms by which neurosensory cells in vertebrates convert mechanical stimuli into electrical signals are unknown.

Among bacteria and invertebrates, several mechanically-gated ion channels have been identified. Stretch-reponsive ion channels have been isolated from bacteria and their molecular structure has been elucidated (Sukharev et al., 1994). A stretchactivated channel was recently reported in yeast (Kanzaki et al., 1999), and a group of genes have been shown by genetic approaches in Caenorhabditis elegans and Drosophila melanogaster to encode putative ion channels involved in mechanosensation (mec-4, mec-10, Osm-9, and NompC; Gu et al., 1996; Lai et al., 1996; Colbert et al., 1997; Walker et al., 2000). Mutations in these genes cause variously touch insensitivity, loss of osmotic avoidance, lack of responsiveness to sound, and dyscoordination through impaired proprioception (Kaplan and Horvitz, 1993; Kernan et al., 1994; Gu et al., 1996; Lai et al., 1996; Colbert et al., 1997; Walker et al., 2000).

Because no genes encoding osmotically or mechanically activated ion channels have been identified in vertebrates, a search was undertaken for mammalian and avian homologues of OSM-9.

In this connection, Delany et al. WO00/32766, published Jun. 8, 2000, disclosed certain human proteins that they named human vanilloid receptor (hVR) proteins, that were determined to play a role in the transmission of pain from sensory neurons to pain-processing centers in the central nervous system. The connection was drawn with the action of capsaicin, and the applicants proposed that the hVR proteins were receptors for vanilloid ligands, which may modulate pain, analgesia, respiratory disorders and inflammatory disorders. Delany et al. identified a particular protein that they named hVR3, that corresponds to the proteins in object herein, however, the applicants neither disclosed nor suggested the particular properties that have been observed herein, and the consequent applications and utilities that have been identified and established by the experiments and data presented herein.

In summary, the field of osmoregulation and particularly, mechanoreception and regulation remain largely unexplained. As the present disclosure will reveal, the identification of legitimate mediators for such activities will result in the development of new and unexpected therapeutic strategies with a broad variety of conditions that are at least partially affected by dysfunctions in either osmotic signal transmission or mechanoreception and signalling.

The citation of any reference herein should not be construed as an admission that such a reference is available as prior art to the application.

### SUMMARY OF THE INVENTION

In accordance with the present invention, a vertebrate receptor that functions as an osmotically gated ion channel, is described that has been named by the present applicants Vanilloid Receptor-Related Osmotically Activated Channel (VR-OAC). VR-OAC has been found to be structurally related to OSM-9 and to the vanilloid receptor 1 (VR1; Caterina et al., 1997), and appears to have significant identity to hVR3 of Delany et al,. supra. The functional properties of this channel that have been observed and determined by the present applicants upon heterologous expression in eukaryotic cells, as well as its gene expression pattern, are unexpected over observations in the literature, as it is suggested herein that it is an osmoreceptor involved in the regulation of systemic osmotic pressure. Equally importantly, and as established by data presented herein, the proteins of the invention are believed to act as mechanoreceptors and thereby could mediate functions where physical movement, touch, and the like are involved. In particular, VR-OAC is involved in inner-ear function, mechanical extero- and interoception, and is expressed in measurable levels in a variety of organs.

Thus, in a first aspect of the invention, a molecule identified as Vanilloid Receptor Related Osmotically Activated Channel (VR-OAC) polypeptide and nucleic acids encoding such polypeptide are set forth herein, and have the sequences presented in FIGS. 1-4. The invention may be extended to oligonucleotides that hybridize to such nucleic acids, antibodies to the polypeptide, and diagnostic and therapeutic compositions and corresponding methods utilizing the polypeptide, nucleic acids, or antibodies, or combinations thereof.

The receptor of the present invention (also termed herein VR-OAC) is characterized by expression at high levels in the lung, spleen, kidney, testis and fat, and expression in lower but significant levels in sensory ganglia, and the inner ear, a finding in line with the concept suggesting and confirming that it may function as an osmoreceptor and as a mechanoreceptor.

In addition to the coding DNA, the present invention provides vectors comprising such DNA. A vector of the invention may be a cloning vector, or it may be an expression vector, which comprises the DNA encoding VR-OAC receptor operatively associated with an expression control sequence. Naturally, the invention extends to an unicellular host transformed or transfected with a DNA molecule, cloning vector, or expression vector of the invention. Such a unicellular host may be selected from the group consisting of bacteria, yeast, mammalian cells, plant cells, and insect cells, in tissue culture. In specific embodiments, the host may be selected from the group consisting of E. coli, Pseudomonas, Bacillus, Streptomyces, Saccharomyces, Pichia, Candida, Hansenula, Torulopsis, CHO, R1.1, B-W, LM, COS 1, COS 7, BSC1, BSC40, BMT10, Sf9 cells and HEK293 cells.

The invention further relates to a recombinant method for preparing a VR-OAC receptor polypeptide comprising culturing a host cell comprising an expression vector of the invention under conditions that provide for expression of the VR-OAC receptor polypeptide; and recovering the expressed polypeptide.

The invention further provides an antisense nucleic acid that hybridizes with an mRNA encoding VR-OAC receptor, and a ribozyme which cleaves an mRNA encoding a VR-OAC receptor.

In another embodiment, the invention provides a transgenic vector comprising a DNA molecule encoding VR-OAC, or an expression vector of the invention. The invention also relates to transgenic animals wherein the expression of VR-OAC is altered, controlled or tagged with an identifier or marker. The invention relates to transgenic animals wherein expression of VR-OAC is enhanced or blocked, so termed VR-OAC overexpressors and VR-OAC knockout animals. Also contemplated are transgenic animals wherein VR-OAC expression is under the control of a promoter or enhancer which may be selectively activated or inhibited. Further contemplated are transgenic animals wherein VR-OAC expression can be monitored or assayed by virtue of an epitope-tagged molecule or marker molecule, which is expressed from the VR-OAC promoter or as a VR-OAC-marker/tag fusion product.

In another aspect, the invention provides an antibody specific for a VR-OAC receptor. The antibody may be a monoclonal, polyclonal or chimeric (bispecific) antibody. Such antibodies include antibodies generated to antigenic fragments of the VR-OAC receptor, including synthetic polypeptide fragments of about 10 to 30 amino acid residues. In a specific embodiment, the antibody may be labeled with a detectable label. Naturally, the invention extends to an immortal cell line that produces a monoclonal antibody.

In a specific embodiment, the invention provides a method for preparing an antibody specific for a VR-OAC receptor, comprising immunizing a host animal with VROAC or an immunogenic fragment thereof admixed with an adjuvant; and obtaining antibody from the immunized host animal. In another specific embodiment, exemplified infra, the method for preparing an antibody specific for a VR-OAC receptor comprises conjugating an immunogenic fragment of a peptide having a sequence selected from the group consisting of FIGS. 2, 4 and 5 to a carrier protein; immunizing a host animal with the peptide-carrier protein conjugate of step (a) admixed with an adjuvant; and obtaining antibody from the immunized host animal.

In conjunction with the antibodies of the invention, the invention provides a method for measuring the presence of a VR-OAC receptor in a sample, comprising contacting a sample suspected of containing a VR-OAC receptor with an antibody that specifically binds to the VR-OAC receptor under conditions which allow for the formation of reaction complexes comprising the antibody and the VR-OAC receptor; and detecting the formation of reaction complexes comprising the antibody and VROAC receptor in the sample, wherein detection of the formation of reaction complexes indicates the presence of VR-OAC receptor in the sample. In a specific embodiment, the antibody is bound to a solid phase support. As a corollary to the method of measuring the presence of VR-OAC receptor in a sample, the invention provides an in vitro method for evaluating the level of VR-OAC receptor in a biological sample comprising detecting the formation of reaction complexes in a biological sample as described; and evaluating the amount of reaction complexes formed, which amount of reaction complexes corresponds to the level of VR-OAC receptor in the biological sample. The invention further relates to an in vitro method for detecting or diagnosing the presence of a disease associated with elevated or decreased levels of VR-OAC receptor in a subject comprising evaluating the level of VR-OAC receptor in a biological sample from a subject as described; and comparing the level detected in step (a) to a level of VR-OAC receptor present in normal subjects or in the subject at an earlier time, wherein an increase in the level of VR-OAC receptor as compared to normal levels indicates a disease associated with elevated levels of VR-OAC receptor, and decreased level of VROAC receptor as compared to normal levels indicates a disease associated with decreased levels of VR-OAC receptor.

The present invention also provides a pharmaceutical composition comprising a soluble VR-OAC receptor, and a pharmaceutically acceptable carrier. Alternatively, a pharmaceutical composition of the invention may comprise a transgenic vector, e.g., a viral vector or naked DNA, for administration to a subject for gene therapy. Preferably, such a vector is targeted to the organ in need of treatment. The invention further provides a method for treating a condition selected from among those having as a primary cause a debilitation or dysfunction in osmotic regulation or mechanoreception in a subject comprising administering a therapeutically effective amount of the pharmaceutical composition of the invention.

Accordingly, it is a principal object of the present invention to provide modulators of osmotic pressure regulation or mechanoreception activity as defined herein in purified form, that exhibit certain characteristics and activities associated with control and variation of the aforementioned and later recited conditions.

It is a further object of the present invention to provide methods for the detection and measurement of the osmotic pressure/mechanoreception modulators as set forth herein, as a means of the effective diagnosis and monitoring of pathological conditions wherein the variation in level of such modulators is or may be a characterizing feature.

It is a still further object of the present invention to provide a method and associated assay system for the screening of substances, such as drugs, agents and the like, that are potentially effective to either mimic, activate or inhibit the activity of VR-OAC, e.g., agonists and antagonists and other modulators of the invention in mammals.

In an related aspect, the invention provides compounds or substances, such as drugs, agents and the like, that are effective to either mimic, activate or inhibit the activity of VR-OAC, e.g., agonists and antagonists.

The invention provides a method for modulating mechanoreception or mechanosensation in a mammal comprising administering to said mammal an effective amount of VR-OAC polypeptide, or active fragments or portions thereof.

The invention further provides a method for modulating mechanoreception or mechanosensation in a mammal comprising administering to said mammal an effective amount of VR-OAC polypeptide, or active fragments or portions thereof wherein said VR-OAC polypeptide comprises the sequence set out in any of SEQ ID NOS: 2, 4, 8 or 9. In a further aspect, the invention provides a method for modulating mechanoreception or mechanosensation in a mammal comprising administering to said mammal an effective amount of VR-OAC polypeptide, or active fragments or portions thereof wherein said active fragment or portion of VR-OAC polypeptide comprises the sequence set not in any of SEQ ID NOS: 5, 6, or 7.

The present invention includes a method for treating a condition characterized by altered mechanoreception or mechanosensation in a mammal comprising administering to said mammal an effective amount of VR-OAC polypeptide, or active fragments or portions thereof, wherein said VR-OAC polypeptide comprises the sequence set out in any of SEQ ID NOS: 2, 4, 8 or 9. The invention further includes a method for treating a condition characterized by altered mechanoreception or mechanosensation in a mammal comprising administering to said mammal an effective amount of VR-OAC polypeptide, or active fragments or portions thereof, wherein said active fragment or portion of VR-OAC polypeptide comprises the sequence set out in any of SEQ ID NOS: 5, 6 or 7.

The invention includes a method for treating a condition characterized by altered mechanoreception or mechanosensation in a mammal, wherein said condition is selected from hearing disorders, vertigo of labyrinthine origin including motion sickness, Meniere disease, neurological disorders (including ataxia due to alterations of afferent input to the CNS, and paraesthesia), male infertility, immune dysfunction with alterations of antigen presentation (including HIV infection), obesity and diabetes mellitus, chronic obstructive lung disorder, bronchial asthma, sexual dysfunction due to sensory deficits, blindness due to corneal or retinal causes, and skin disorders (including psoriasis, pemphigus vulgaris and other forms of pemphigoids, pruritus, allergic skin diseases).

The invention provides in a further aspect a method for modulating mechanoreception or mechanosensation in a mammal comprising introducing to said mammal a nucleic acid vector capable of expressing an effective amount of VROAC polypeptide, or active fragments or portions thereof, wherein said VR-OAC polypeptide comprises the amino sequence set out in any of SEQ ID NOS: 2, 4, 8 or 9. In an additional aspect, the invention provides a method for modulating mechanoreception or mechanosensation in a mammal comprising introducing to said mammal a nucleic acid vector capable of expressing an effective amount of VROAC polypeptide, or active fragments or portions thereof, wherein said active fragment or portion of VR-OAC polypeptide comprises the sequence set not in any of SEQ ID NOS: 5, 6, or 7.

The present invention additionally provides a method for determining whether a subject is suffering from altered mechanoreception or mechanosensation comprising determining the expression of VR-OAC polypeptide or ribonucleic acid capable of encoding VR-OAC polypeptide.

In one aspect the method for determining whether a subject is suffering from altered mechanoreception or mechanosensation comprising the steps of:

a) contacting a sample from a subject for which altered mechanoreception or mechanosensation is suspected with an antibody to the VROAC polypeptide under conditions that allow binding of the VR-OAC polypeptide to the antibody to occur; and

b) detecting whether binding has occurred between the VR-OAC from the sample and the antibody; wherein the detection of binding indicates that presence or activity of the VR-OAC polypeptide in the sample.

The invention further provides a method of screening for modulators of mechanoreception or mechanosensation comprising the steps of:

a) contacting a sample in the presence of a candidate modulator with an antibody to the VR-OAC polypeptide under conditions that allow binding of the VR-OAC polypeptide to the antibody to occur; and

b) detecting whether binding has occurred between the VR-OAC from the sample and the antibody;

wherein the detection of binding indicates that presence or activity of the VR-OAC polypeptide in the sample.

The present invention includes a method of screening for modulators of mechanoreception or mechanosensation comprising the steps of:

a) contacting a C. elegans osm-9 mutant which expresses VR-OAC polypeptide with a candidate modulator; and

b) assessing the activity of VR-OAC in the presence of said modulator by determining nose touch sensitivity and/or osmotic avoidance in said C. elegans mutant.

In a further aspect, the invention provides a biosensor or nanotechnological device, which comprises as one of its components the VR-OAC polypeptide or active fragments or portions thereof.

The biosensor or technological device of the present invention includes a biosensor or technological device wherein said VR-OAC polypeptide or active fragments or portions thereof comprises the amino acid sequence set out in any of SEQ ID NOS: 2, 4, 5, 6, 7, 8 or 9.

In an additional aspect of the invention, is provided the use of compounds or substances, such as drugs, agents and the like, that are effective to either mimic, activate or inhibit the activity of VR-OAC, e.g., agonists and antagonists, and other modulators of the invention in mammals in modulating VR-OAC activity for the treatment, alleviation or prophylaxis of a disorder which is responsive to the modulation of VR-OAC activity.

It is a still further object of the present invention to prepare genetic constructs for use in genetic therapeutic protocols and/or pharmaceutical compositions for comparable therapeutic methods, which comprise or are based upon one or more of the modulators, binding partners, or agents that may control their production, or that may mimic or antagonize their activities.

Other objects and advantages will become apparent to those skilled in the art from a review of the ensuing description which proceeds with reference to the following illustrative drawings.

### BRIEF DESCRIPTION OF THE DRAWINGS

FIG. 1 is the nucleic acid sequence of human VR-OAC as determined from HEK293 cells.

FIG. 2 is the amino acid sequence of human VR-OAC as determined from HEK293 cells.

FIG. 3 is the amino acid sequence for human VR-OAC as determined from human kidney.

FIG. 4 is the nucleic acid sequence for human VR-OAC as determined from human kidney.

FIG. 5—Analysis of VR-OAC amino acid sequences

Comparison of the amino acid sequences for VR-OAC from the rat (Rn; 871 amino acids), mouse (Mm; 873 amino acids), human (Hs; 871 amino acids), and chicken (Gg; 852 amino acids) with related proteins, including RnVR1, RnVRL-1, Caenorhabditis elegans (Ce) OSM-9 (GenBank accession number AF031408), and its putative Drosophila melanogaster (Dm) orthologue CG4536 (GenBank accession number AAF46203). Amino acid residues are numbered from the first methionine of RnVR-OAC. The amino and carboxyl termini of RnVR1, RnVRL-1, OSM-9, and CG4536 that do not align with the VR-OACs are omitted. Lower-case letters denote the first and last residues of insertions with respect to the RnVR-OAC sequence. Red columns highlight positions with identical residues over all sequences. Blue columns indicate positions with identical residues within sequence groups (the VR-OACs, RnVR1 and RnVRL-1, and OSM-9 and CG4536). Cyan columns denote conserved positions. The last alignment row shows the consensus sequence. The lower-case characters indicate conservation of chemical classes: o=alcohol, 1=aliphatic, a=aromatic, c=charged, h=hydrophobic, p=polar, s=small, u=tiny, and t=turnlike. The percentage of sequence identity of each sequence to the RnVR-OAC sequence is shown at the end of the alignment. Ankyrin-repeat domains (ARD, pale blue boxes), transmembrane regions predicted by PHDhtm (TM, magenta boxes), putative pore-loop regions (PL, gray box), and the secondary structures predicted for RnVR-OAC by PHDsec are indicated. The triangle indicates a putative cAMP-dependent phosphorylation site, open circles denote predicted PKC phosphorylation sites, and filled circles indicate possible asparagine glycosylation sites.

FIGS. 6A-6B—Schematic structure and phylogenetic relations of VR-OAC

(A) Schematic structure of VR-OAC predicted by hydropathy analysis. Three ankyrin-repeat domains (ARD) occur near the amino terminus. The channel's core comprises six α-helical transmembrane domains (TM) and a pore loop (PL).

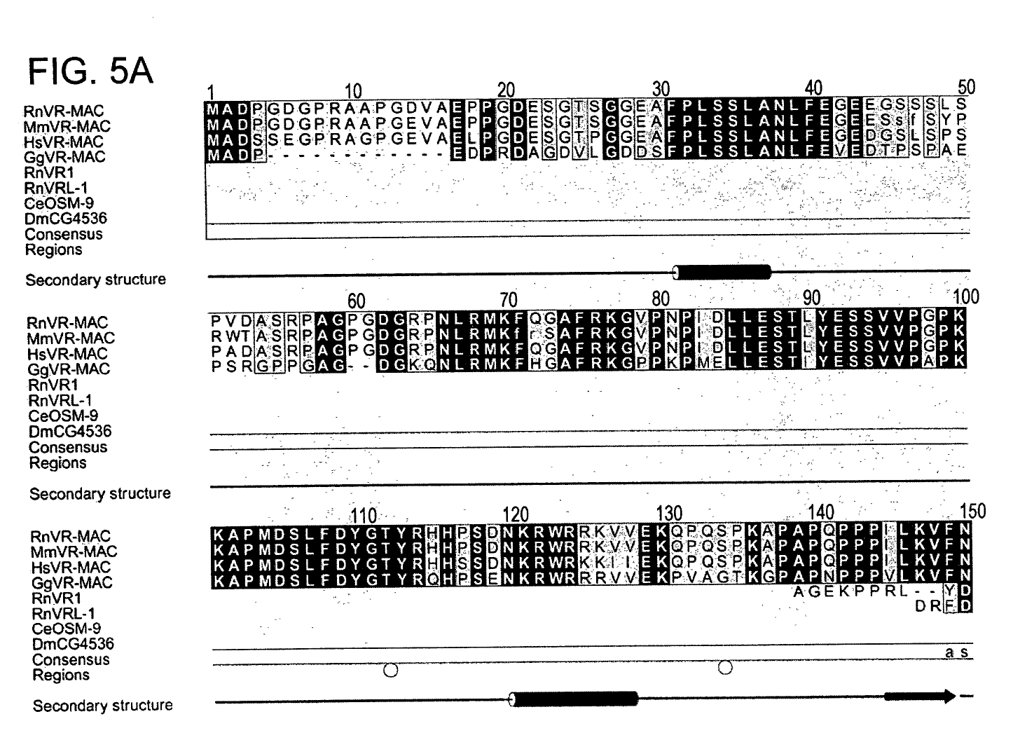

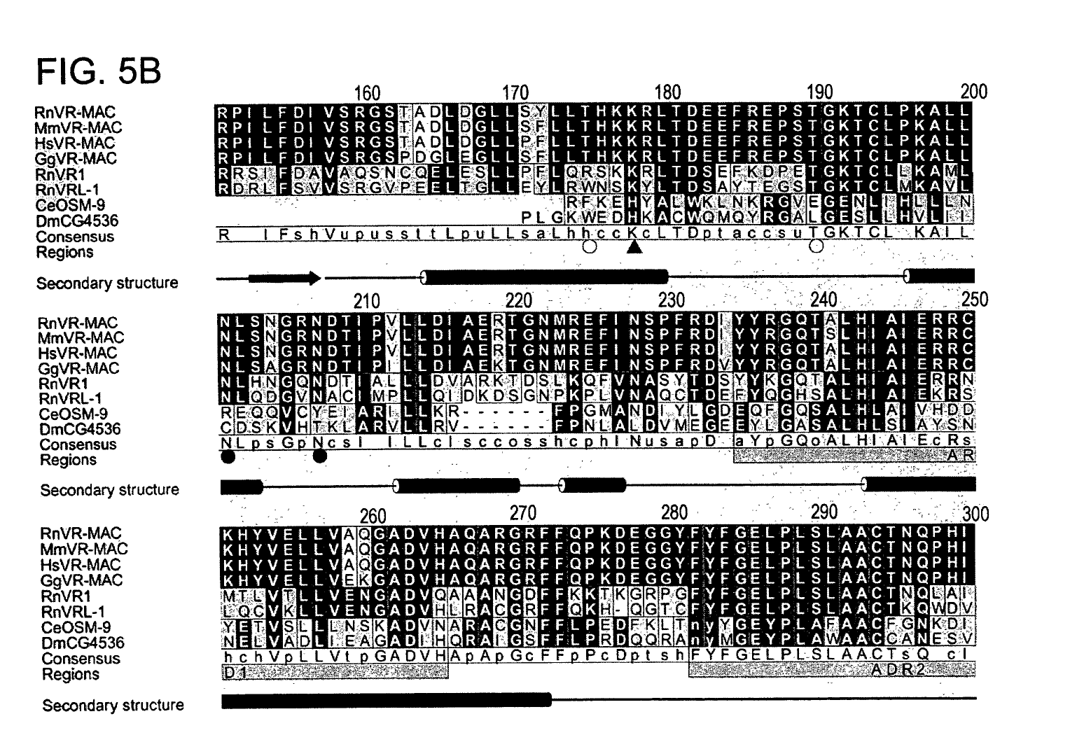

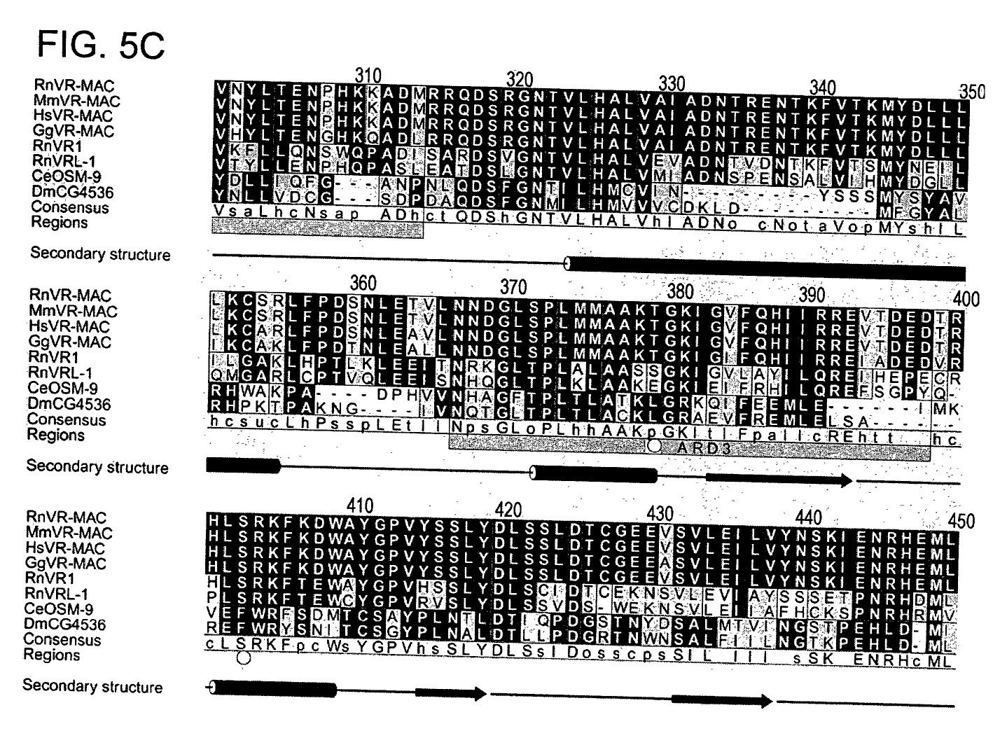

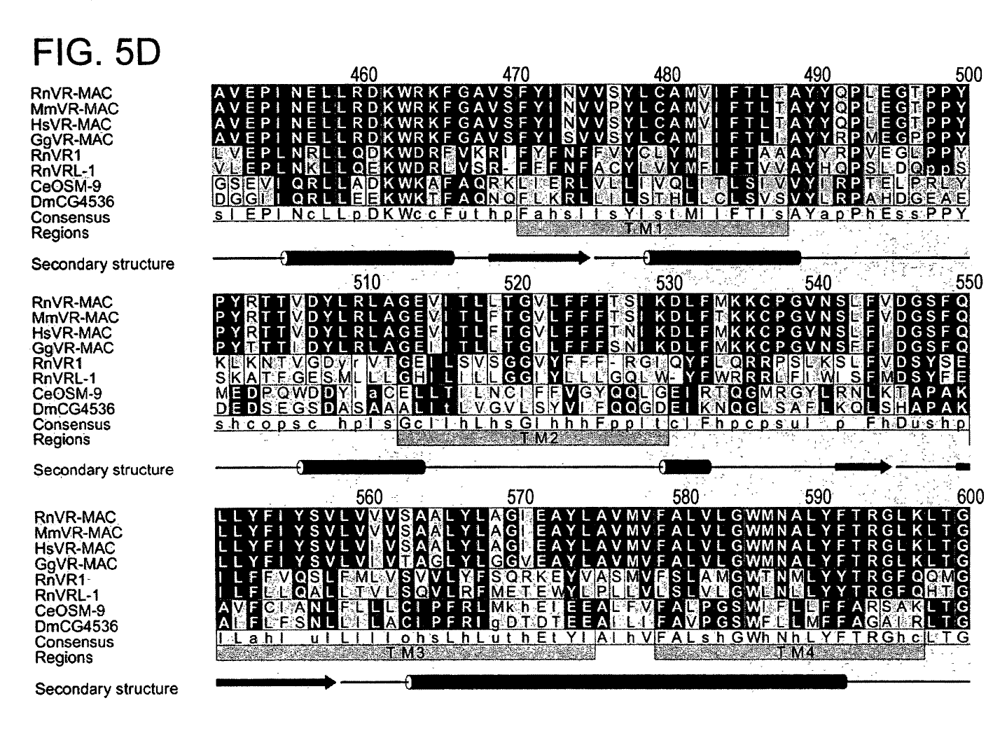

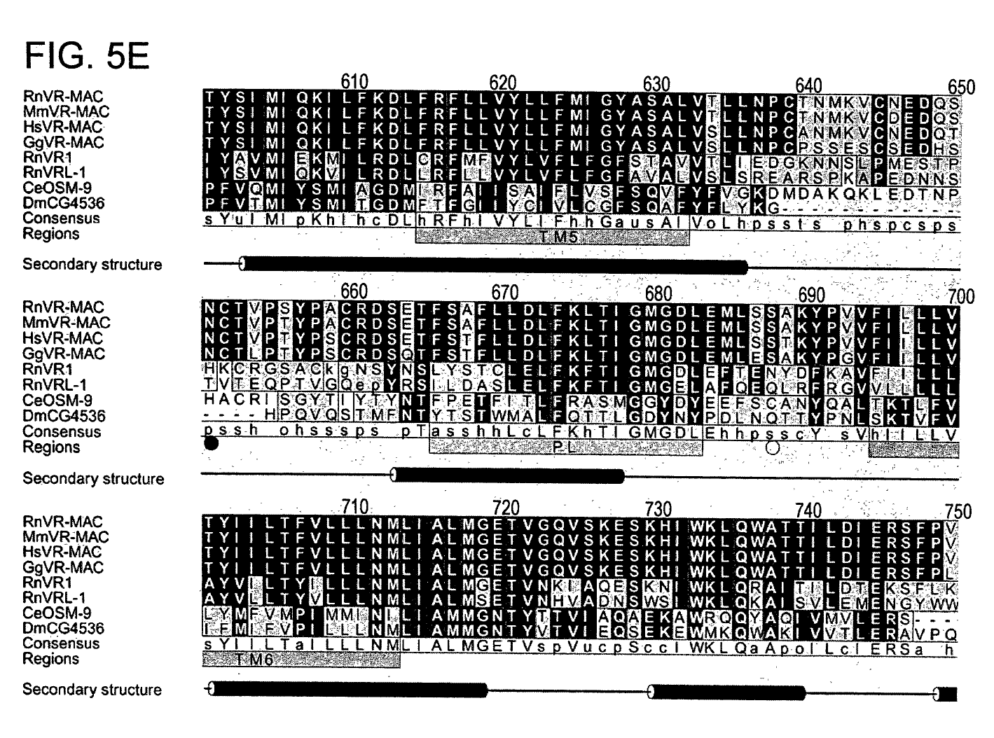

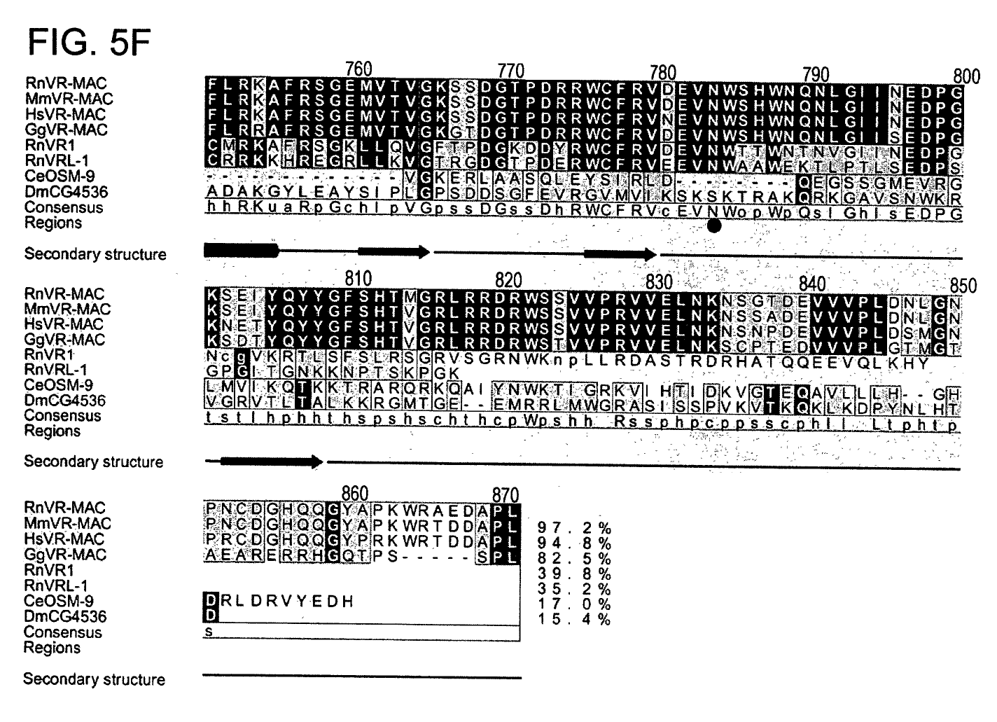

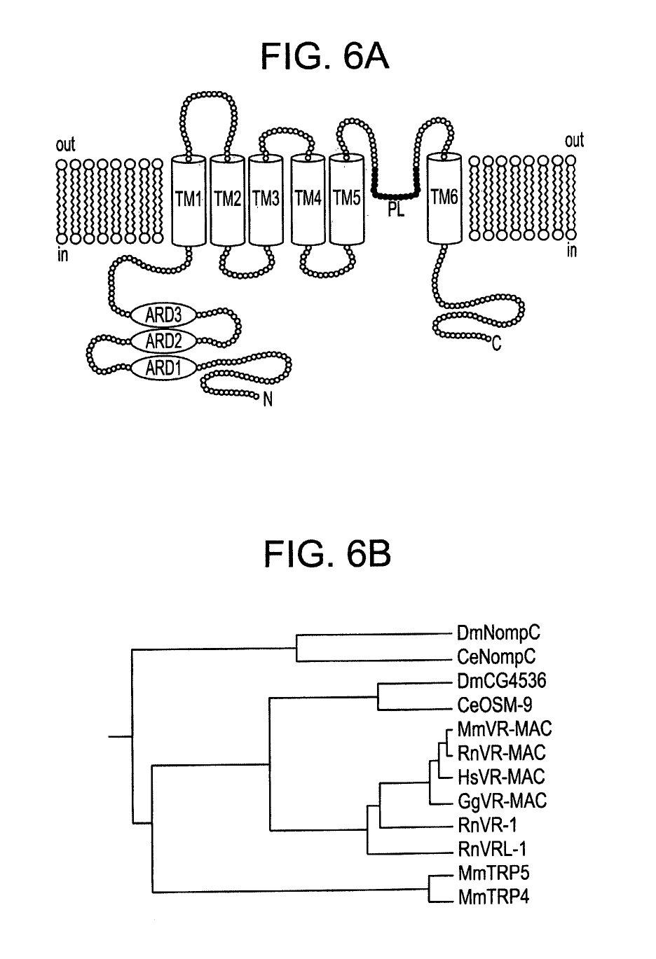

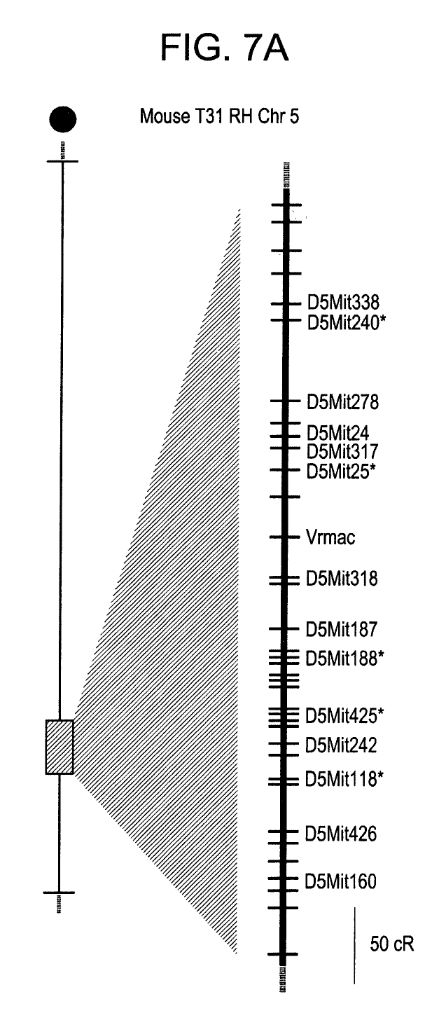

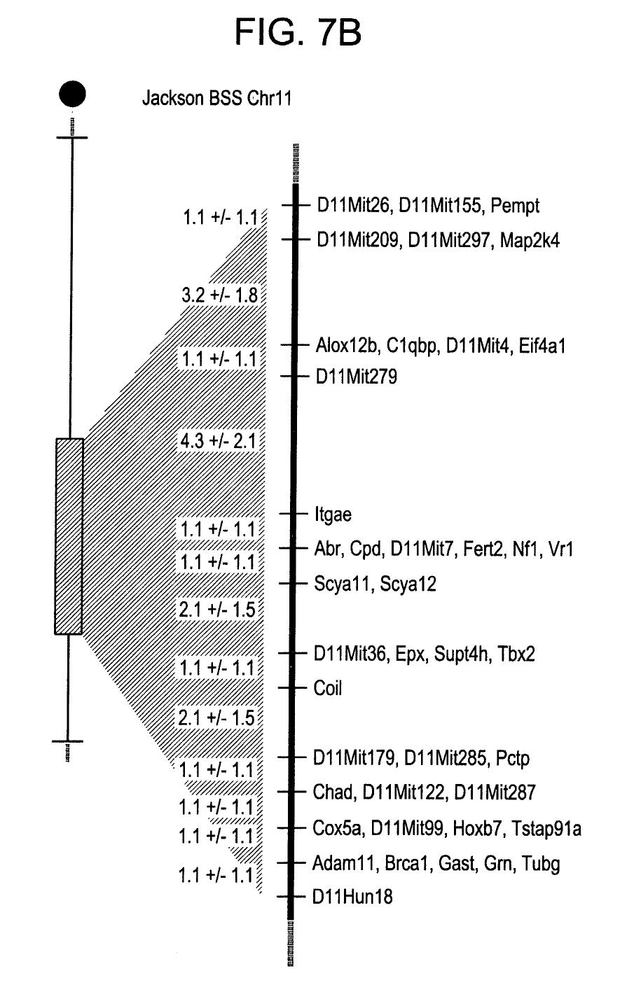

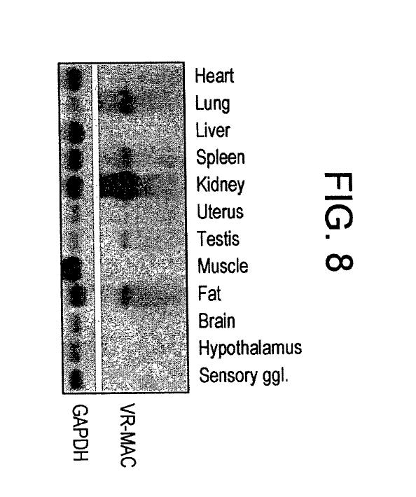

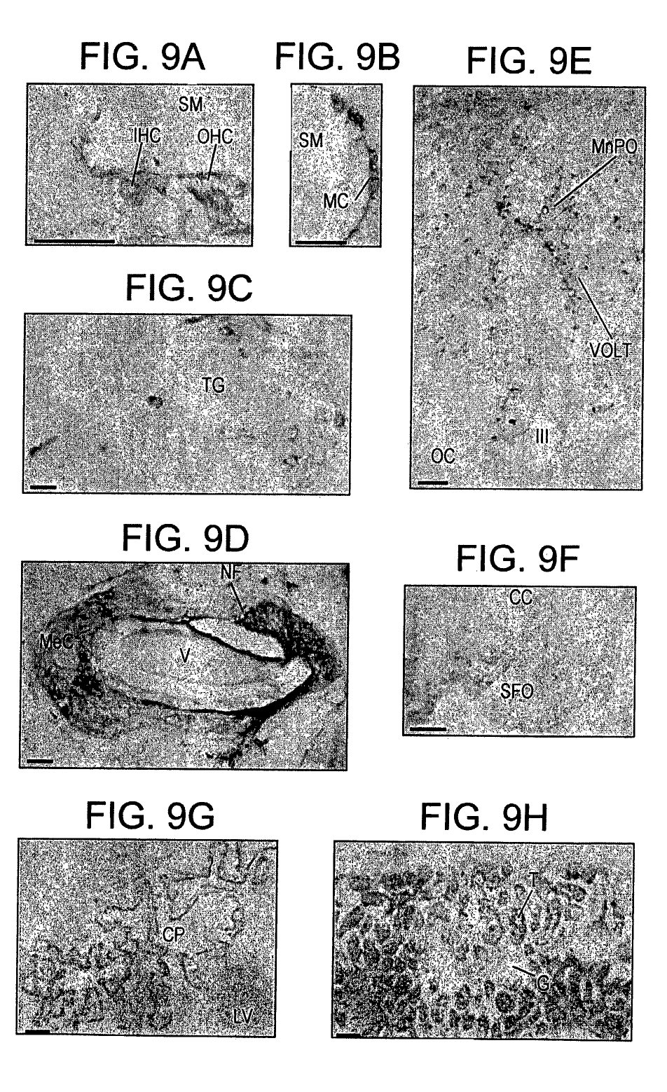

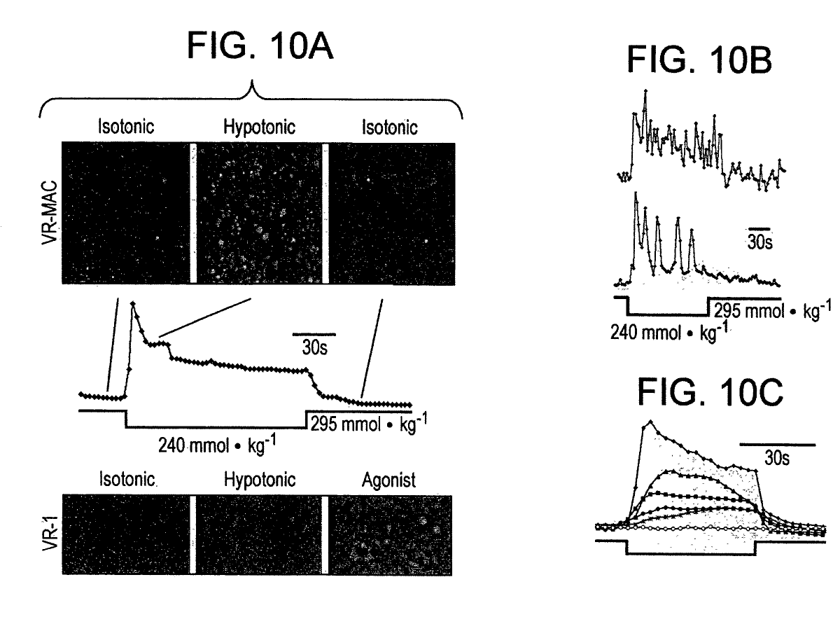

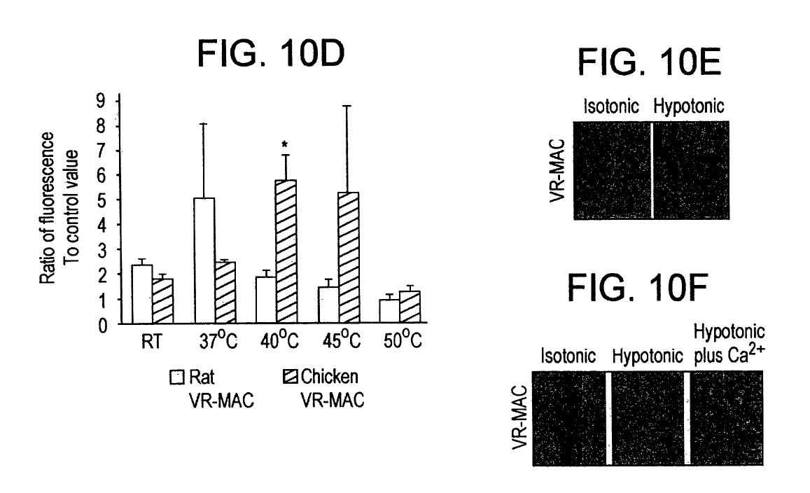

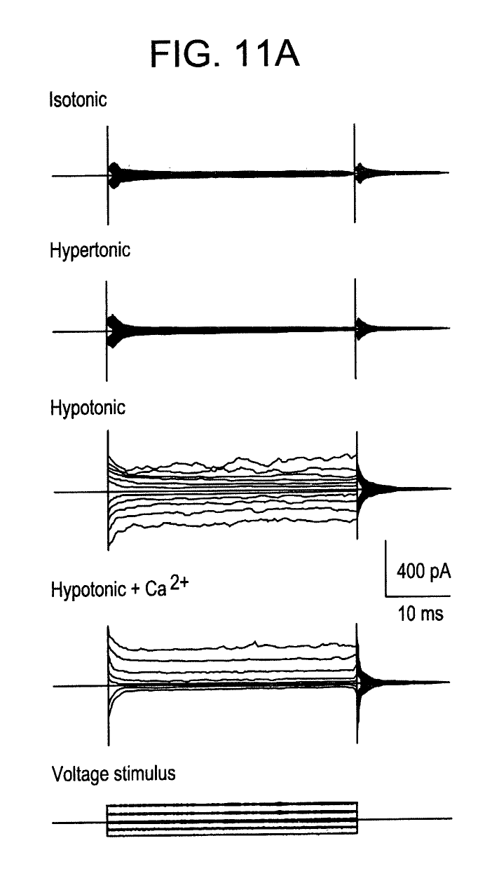

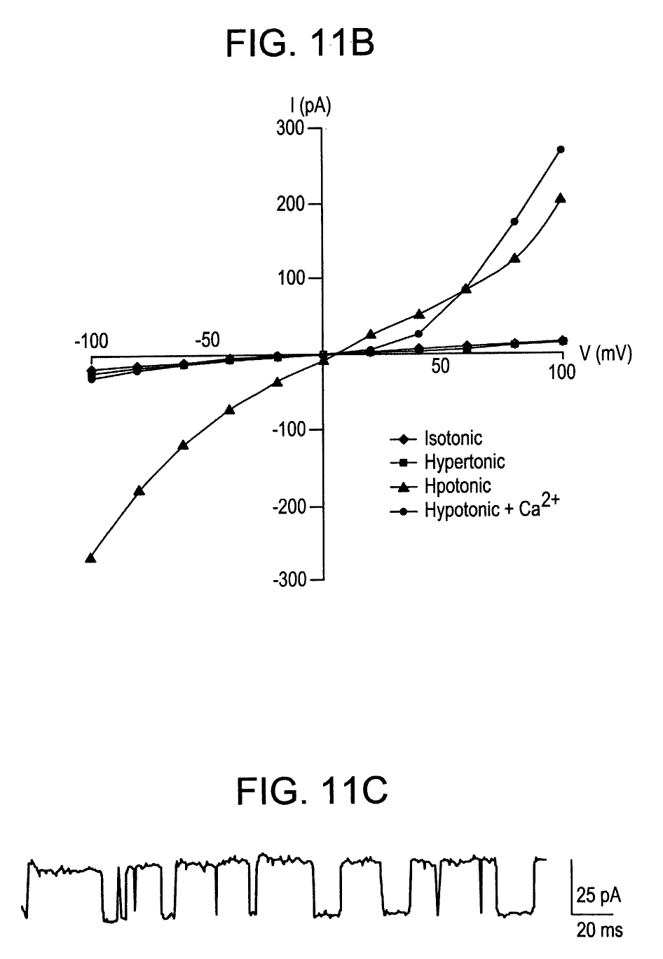

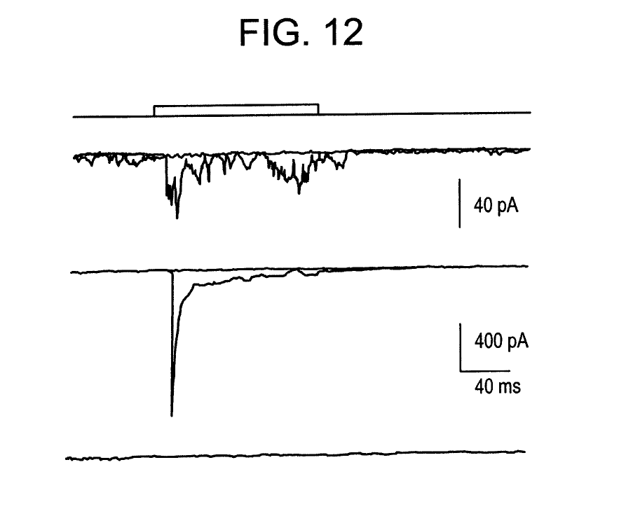

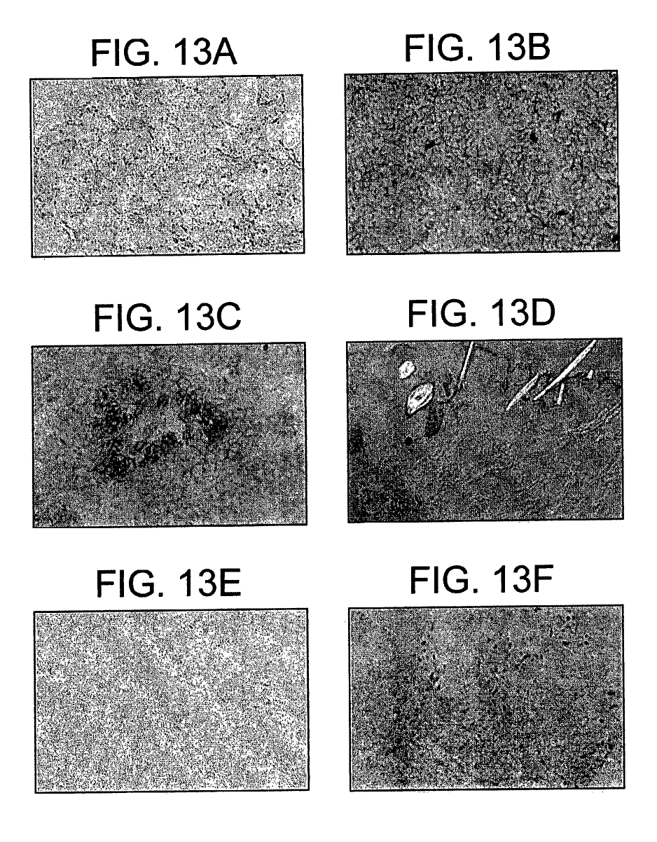

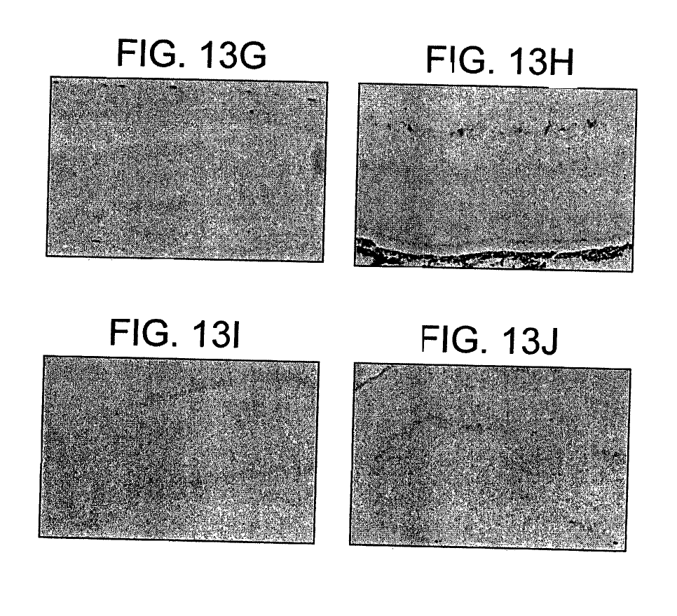

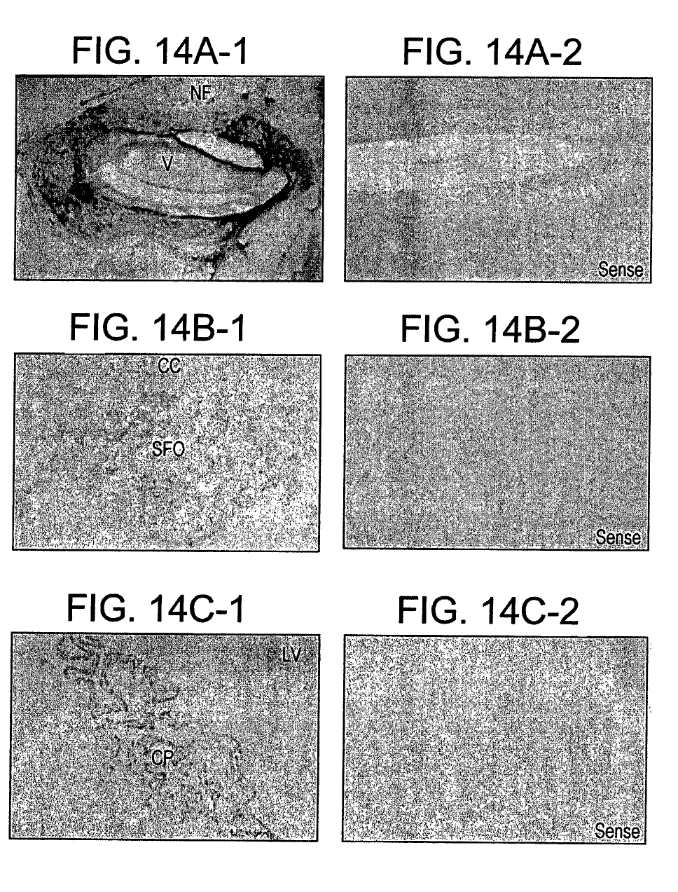

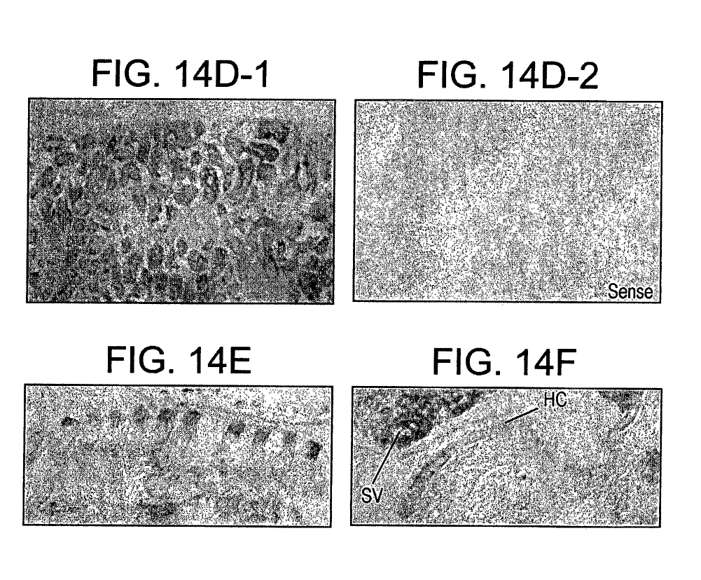

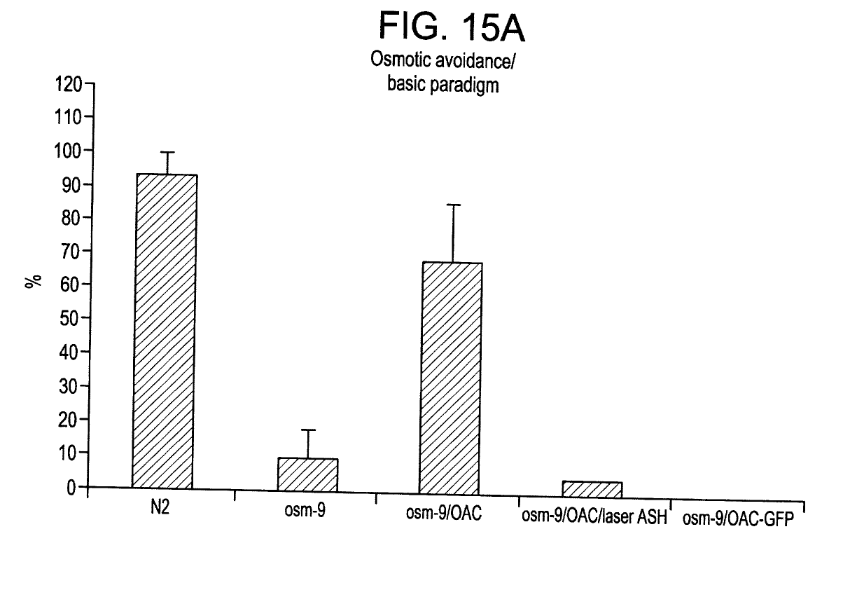

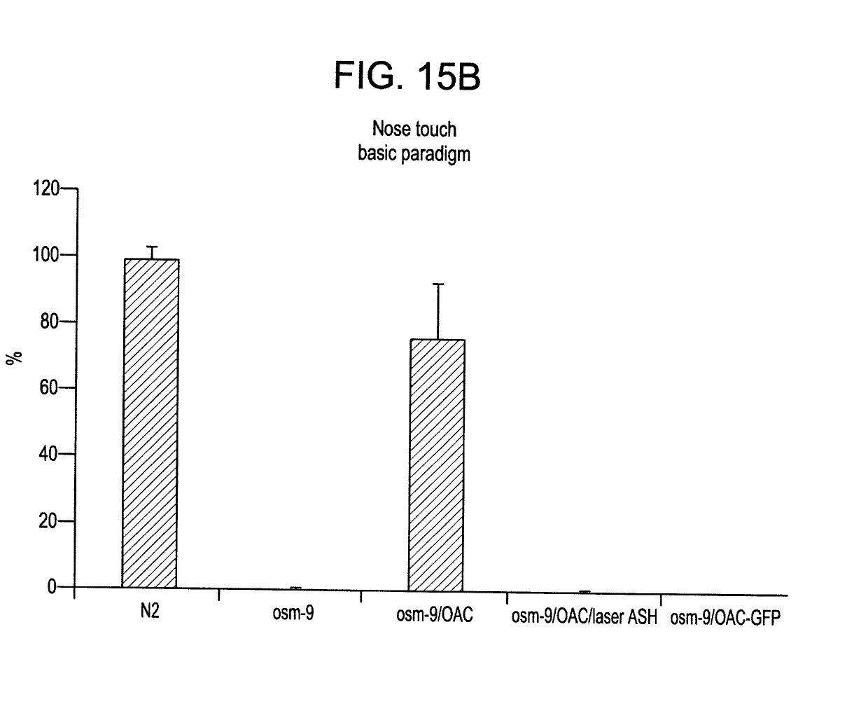

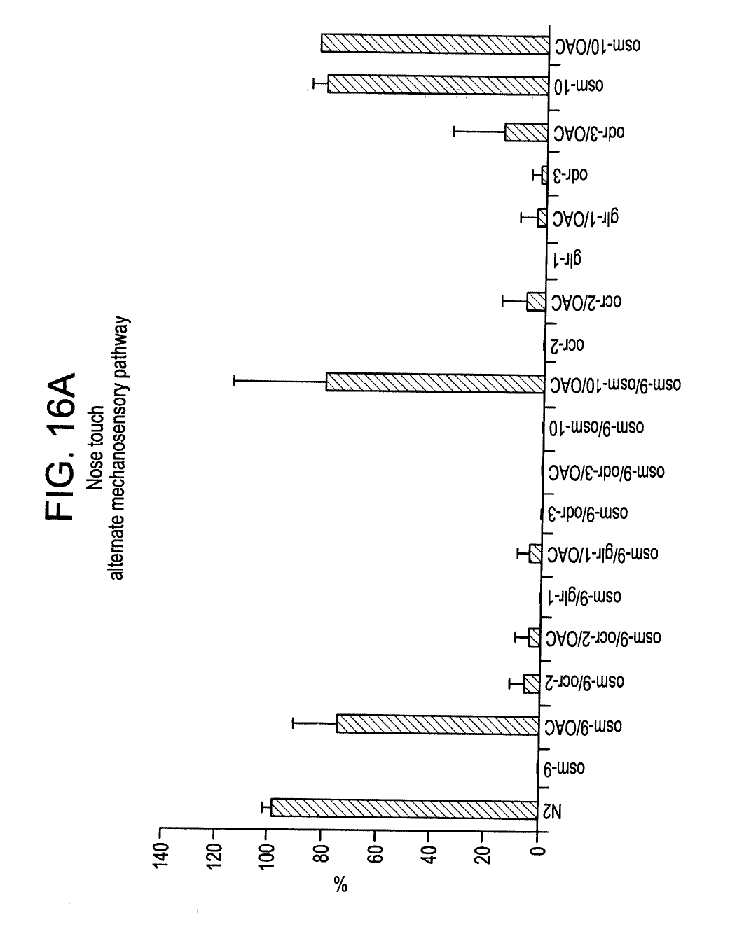

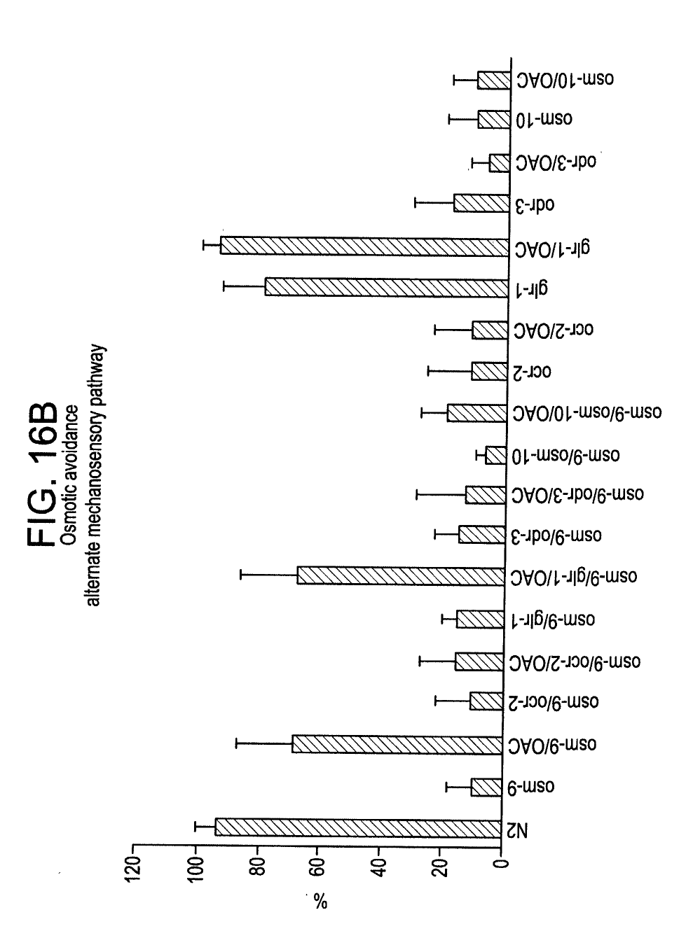

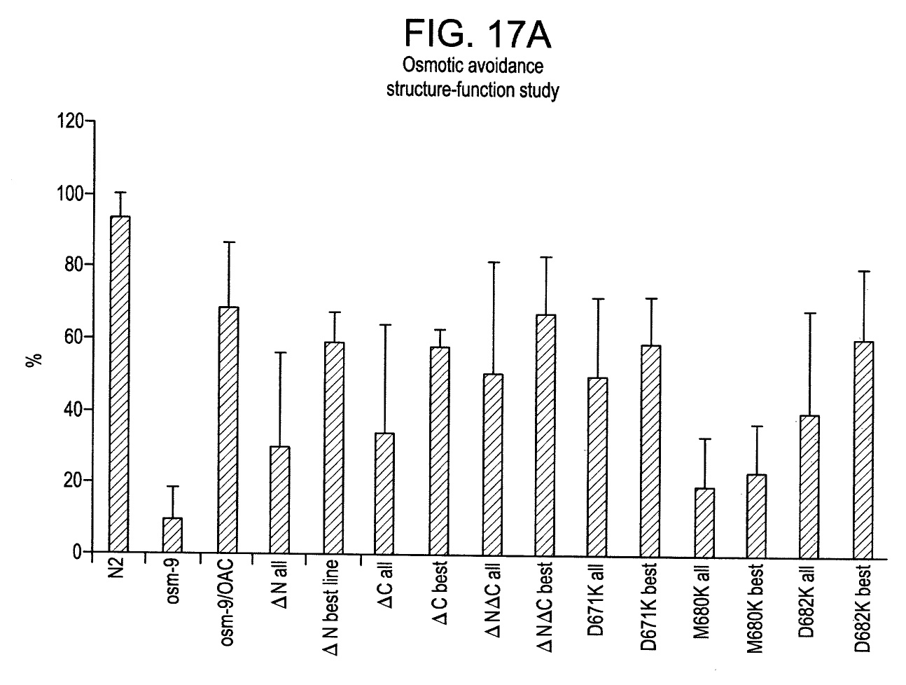

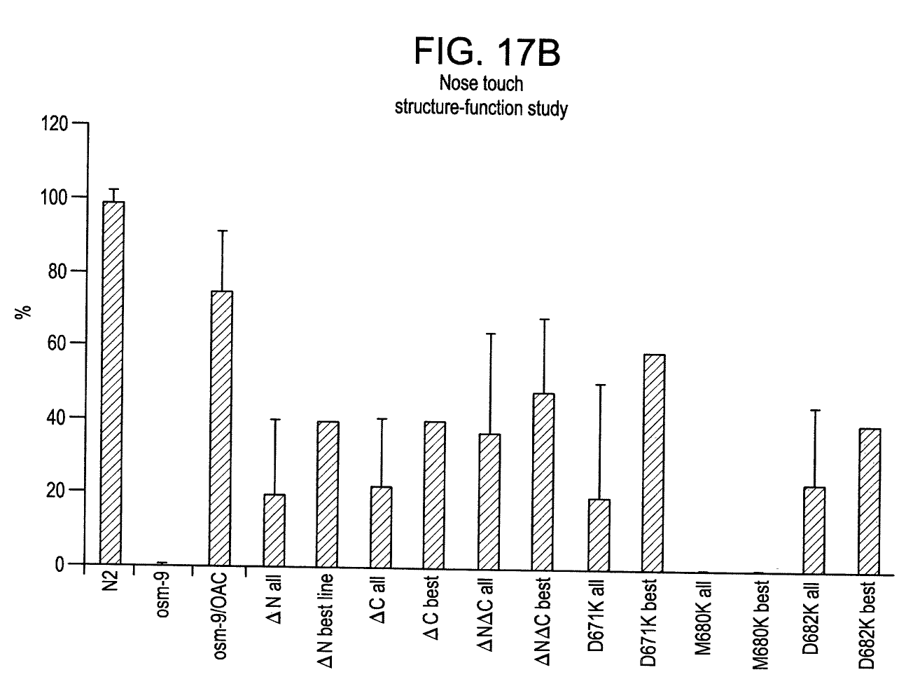

(B) The phylogenetic relations among VR-OAC-related proteins, including NOMPC and mammalian TRP proteins. The species abbreviations are provided in the caption of FIG. 5.

FIG. 7-VR-OAC mRNA expression in rat organs

A multiple-organ Northern blot demonstrates expression of a 3.2-kb VR-OAC mRNA in lung, spleen, kidney, testis, fat, and faintly in trigeminal ganglia. The upper panel shows an autoradiograph of the membrane hybridized with a VR-OAC-specific probe. As an indicator of the relative mRNA loading, the lower panel shows the signal after hybridization to detect the mRNA of glyceraldehyde 3-phosphate dehydrogenase.

FIGS. 8A-8D—In situ hybridization analysis of VR-OAC expression in the central nervous system

(A) In a coronal section of the lamina terminalis of the mouse brain, VR-OAC-expressing neurons occur in the arched vascular organ of the lamina terminalis (VOLT). Positive neurons are also located in the median preoptic area (MnPO); a few labeled neurons are scattered through the adjacent brain. The optic chiasm (OC) lies below the third ventricle (III), whose ependymal cells are unlabeled.

(B) In another coronal section of the murine lamina terminalis, VR-OAC mRNA is abundantly expressed in neurons of the subfornical organ (SFO). VHC, ventral hippocampal commissure.

(C) The ependymal cells of the choroid plexus (CP) of the rat's lateral ventricle (LV) express VR-OAC mRNA. CC, corpus callosum.

(D) Two orientation drawings situate the structures in panels A-C in coronal sections of the rodent brain. The abbreviations are as noted for those illustrations. The sections in panels A-C were lightly counterstained with nuclear fast red. The scale bars correspond to 50 μm.

FIGS. 9A-9E—In situ hybridization analysis of rodent VR-OAC expression

(A) In the murine cochlea, VR-OAC mRNA occurs in both inner hair cells (1HC) and outer hair cells (OHC). SM, scala media.

(B) Marginal cells (MC) of the cochlear stria vascularis in the mouse display VR-OAC mRNA.

(C) In the murine trigeminal ganglion (TG), VR-OAC mRNA occurs in a population of large neurons. Specific staining is not detectable in small and very large sensory ganglion cells.

(D) Surrounding the obliquely sectioned shaft of a vibrissa (V) from an albino rat's snout, Merkel cells (MeC) strongly express VR-OAC (blue reaction product). Nerve fibers (NF) innervating the Merkel cells are black following anti-neurofilament immunolabeling.

(E) In the cortex of the murine kidney, VR-OAC is strongly expressed by epithelial cells of tubules (T). The expression in glomeruli (G) is much weaker. The scale bars correspond to 50 μm.

FIGS. 10A-10F—Gating of VR-OAC investigated by Ca²⁺ imaging

(A) CHO cells, permanently transfected with an expression vector for chicken VR-OAC and loaded with the Ca²⁺-indicator fluo-4, are observed by confocal microscopy. Replacement of the isotonic extracellular solution (295 mmol kg⁻1) with hypotonic medium (245 mmol kg-1) results in a dramatic increase in fluorescence, reflecting a rise in the intracellular Ca²⁺ concentration (upper panels). Replacement of isotonic solution restores the Ca²⁺ concentration to its background level. In a quantitative analysis of frames from the series, each point represents the fluo-4 fluorescence from a microscopic field containing approximately 2000 cells (plot). The peak fluorescence is 3.8× as great as the control value. Control cells expressing rat VR1 do not exhibit alterations of intracellular Ca²⁺ concentration when the osmotic strength is changed (lower panels). Exposure to 200 nM of the vanilloid agonist resiniferatoxin increases the intracellular Ca²⁺ concentration.

(B) Individual cells respond to hypotonic solution either by an elevated Ca²⁺ concentration throughout the stimulus period (upper trace) or by an oscillatory increase (lower trace). The peak fluorescence for each experiment is 5× as great as the respective control value.

(C) Cells stably transfected with chicken VR-OAC produce graded responses to a range of hypotonic solutions. The data points represent an exchange from isotonic solution (295 mmol kg⁻¹) to solutions with osmotic strengths (in mmol kg⁻¹) of 223 (diamonds), 247 (triangles), 259 (squares), 273 (filled circles), 288 (stars), and 295 (open circles). The stimulus period is indicated below the traces. The peak fluorescence is 4.6× as great as the control value.

(D) The temperature sensitivity of cell lines stably transfected with rat or chicken VR-OAC is demonstrated by the fluorescence from roughly 2000 cells stimulated with hypotonic solution of 260 mmol kg⁻¹. Data are presented as means and standard deviations from 3-4 measurements. For rat VR-OAC, the sensitivity peaks at 37° C., the mammalian core body temperature; for chicken VR-OAC, maximal responsiveness occurs at 40° C., the avian core body temperature. RT, room temperature.

(E) In a control experiment, internal Ca²⁺ stores are depleted with 10 μM thapsigargin and potentiation channels are blocked with 20 μM SKF 96365. Under these conditions, the modest background level of fluo-4 fluorescence does not increase upon exposure to hypotonic medium.

(F) When transfected cells in isotonic medium free of Ca²⁺ are exposed to Ca²⁺ free hypotonic medium, no change occurs in the intracellular fluo-4 fluorescence. When 2 mM Ca²⁺ is added to the medium, however, the intracellular Ca²⁺ concentration promptly rises.

FIGS. 11A-11C—Electrophysiological characterization of VR-OAC-expressing CHO cells

(A) Whole-cell current responses to voltage-step stimuli illustrate the osmotic sensitivity and Ca²⁺-dependent rectification of VR-OAC. The membrane potential was held at 0 mV and stepped in 20-mV increments to ±100 mV (bottom family of traces). Cells exposed to isotonic or hypertonic solutions responded similarly to untransfected control cells. Hypotonic solutions, however, elicited robust whole-cell currents with marked outward rectification in the presence of 1 mM free Ca²⁺. The rectification developed rapidly and disappeared immediately upon withdrawal of Ca²⁺.

(B) The voltage-current relation under hypotonic conditions displays dual rectification. Inclusion of Ca²⁺ in the hypotonic medium significantly reduces the inward current.

(C) A current record from an inside-out patch at +80 mV shows unitary events corresponding to a conductance of 310 pS. The upper level represents the channel's open state.

Although the results shown were taken from chicken VR-OAC recordings, the electrophysiological responses of the rat orthologue corroborated the principal conclusions.

FIG. 12 depicts the current elicited by exposure of a transfected cell to a fluid jet. The upper traces represent the electrical signal controlling the fluid-jet pulse. Each of the lower three pairs of recordings, taken sequentially from the same cell, includes the baseline current in a control trace as well as the response to stimulation. The membrane potential is held at −60 mV. Note that the current scale for the first pair of current recordings differs from that in the latter two pairs. Similar responses were obtained in five of ten cells examined by this paradigm.

FIGS. 13A-13J comprises a panel of photographs presenting expression data confirming the presence of VR-OAC in the following compartments/organs: The panel shows in situ hybridization of rodent tissue samples with nucleotide probes specific for VR-OAC. A-J are as follows.

A. Mouse lung. VR-OAC is expressed in lung tissue, in alveolar cells.

B. Mouse spleen. VR-OAC is strongly expressed in cells resembling macrophages and follicular dendritic cells, key antigen presenting cells of the immune system.

C. Rat testis. VR-OAC is heavily expressed in spermatocytes.

D. Rat snout skin. VR-OAC is expressed in Merkel cells in the sinus of vibrissae and also in the vicinity of smaller hairs. VR-OAC is also expressed in touch-sensitive Merkel cells associated with epidermis and in epidermal cells.

E. Rat white adipose tissue. VR-OAC is expressed in adipocytes.

F. Mouse orbital tissue. VR-OAC is expressed in orbital adipocytes.

G. Mouse cornea. VR-OAC is expressed in corneal squamous epithelial cells. It was also found in the angle of the anterior chamber of the eye (not shown).

H. Mouse retina. VR-OAC is expressed in photoreceptors and retinal ganglion cells.

I. Mouse brain. VR-OAC is expressed in nerve-cells of the hippocampus, CAl region, a region of importance for memory and in epileptic seizures.

J. Mouse brain. VR-OAC is expressed in cerebellar nerve cells. All sections with mouse tissue have been recapitulated with rat tissue and vice versa. With a protein sequence similarity of 94.8% between rat and human, a similar gene expression profile can be reasonably assumed to be detected in human tissue.

FIGS. 14A-14F are photographs demonstrating additional expression data confirming the presence of VR-OAC in the following compartments/organs:

A, A′) Albino rat snout vibrissa (V)

A) An in situ hybridization with a VR-OAC antisense cRNA. The blue specific signal can be detected in Merkel cells (MeC). The black deposit stems from immunolabeling for neurofilament protein.

A′) No specific signal is obtained with a sense control cRNA.

No counterstain.

B, B′) Mouse central nervous system, subformical organ

B) In an in situ hybridization with a VR-OAC antisense cRNA, specific labeling can be detected in neurons of the subformical organ (SFO). For orientation: corpus callosum (CC).

B′) No specific signal occurs when a sense control cRNA is used.

Light counterstain with nuclear fast red.

C, C′) Rat central nervous system, lateral ventricle (LV) with choroid plexus (CP)

C) In an in situ hybridization with a VR-OAC antisense cRNA, specific labeling is detected in ependymal cells of the choroid plexus. No counterstain.

C′) No specific signal occurs when a sense control cRNA is used. Light counterstain with nuclear fast red.

D, D′) Mouse renal cortex

D) In an in situ hybridization with a VR-OAC antisense cRNA, a specific signal is detectable in tubular epithelial cells (T) and to a much lesser extent in glomeruli (G).

D′) No specific signal is detected when a sense control cRNA was employed.

Light counterstain with nuclear fast red.

E) Longitudinal section, mouse inner ear, organ of Corti

An in situ hybridization with a mouse VR-OAC antisense cRNA shows intense apical labeling in tangentially sectioned outer hair cells. No counterstain.

F) Chicken inner ear, cochlea

In an in situ hybridization of the chicken's cochlea with chicken VR-OAC antisense cRNA, a specific signal occurs in hair cells (HC) and in cells of the tegmentum vasculosum (TV), the chicken equivalent of the mammalian stria vascularis. No counterstain.

FIGS. 15A and 15B depict assessment of (A) osmotic avoidance and (B) nose touch in C. elegus wild type, mutant and recombinant strains. N2=wild type, osm-9=osm-9 mutant, osm-9/OAC=osm-9 mutant expressing VR-OAC, osm9/OAC/laser ASH=osm-9 mutant expressing VR-OAC where both ASH neurons have been laser ablated, osm-9/OAC-GFP=osm-9 mutant expressing GFP-tagged VR-OAC.

FIGS. 16A and 16B depict assessment of (A) nose touch and (B) osmotic avoidance in various C. elegans mutants, specifically single mutants, double mutants, and single or double mutants expressing VR-OAC.

FIG. 17A OR 17B depict assessment of (A) osmotic avoidance and (B) nose touch in various C. elegans mutants. ΔN indicates N-terminal VR-OAC deletion lacking amino acids 1-410 of VR-OAC, ΔC indicates C-terminal VR-OAC deletion lacking amino acids 741-781 of VR-OAC, ΔNΔC is a VR-OAC deletion of both N-terminal and C-terminal regions as above noted, D671K is a VR-OAC mutation of Lysine for Aspartic Acid at amino acid 671, D682K is a VR-OAC mutation of Lysine for Aspartic Acid at amino acid 682, M680K is a VR-OAC mutation of Lysine for Methionine at amino acid 680.

### DETAILED DESCRIPTION OF THE INVENTION

The present invention relates to the elucidation of a protein termed herein VR-OAC and to the observations as to its activities as a osmotic pressure sensitive and mechanical stimulation-sensitive factor. The invention includes the protein, nucleic acids encoding the protein, and the uses to which the protein and analogs thereof may be put, relating to the diagnosis and treatment of a variety of conditions, among them, the following non-limiting listing: disabilities and dysfunctions associated with sensory organs, neurological disorders, hearing disorders, kidney disorders, male infertility, immune dysfunction, obesity and diabetes mellitus, eye diseases, skin disorders, lung disorders and bronchial asthma.

With respect to reference to the proteins and the nucleic acids associated or corresponding to VR-OAC, it should be noted that where all capitals are used it refers to the natural protein or gene; all lower case refers to a mutant protein or gene; italics indicates a gene or nucleic acid molecule; and normal type indicates a protein or polypeptide), including degenerate variations thereof, e.g., that incorporate optimal codons for expression in a particular expression system, which protein demonstrates the ability to act as an osmoreceptor and a mechanoreceptor.

The VR-OAC of the invention contains six membrane spanning domains and a pore loop (FIG. 6), and an amino-terminal domain with three ankyrin repeats, that occurs intracellularly. The VR-OAC polypeptide of the present invention comprises the amino acid sequence as set out in any of SEQ ID NOS: 2, 4, 5, 6, 7, 8 and 9. The VR-OAC polypeptides of the present invention include VR-OAC polypeptide, mutants thereof and active fragments or portions thereof.

In its primary aspect, the present invention is directed to the application of VR-OAC as a modulator of osmoreception and functionality, and similarly, with respect to mechanoreception and functionality. Accordingly, the receptor of the invention may be employed in methods for the examination and testing of certain organ systems to determine their condition and to diagnose any pathologies or dysfunctions that may be manifested in the presence and/or activity of VR-OAC. Likewise, assays may be prepared and used for the identification of agents that may exhibit the activity of VR-OAC, and to thereby develop new drugs or diagnostic/prognostic indicators. Such an assay may comprise a colony of cells from a particular organ, such as from the inner ear, where eg. cells having measurable levels of VR-OAC, may be present, so that the introduction of a potential agent/drug with appropriate labeling, may be observed and its activity, if any, assessed for possible selection and further efficacy testing. Further, an assay may be constructed that will test cells taken from an organ system for VR-OAC activity to thereby assess the well being and functionality of the organ.

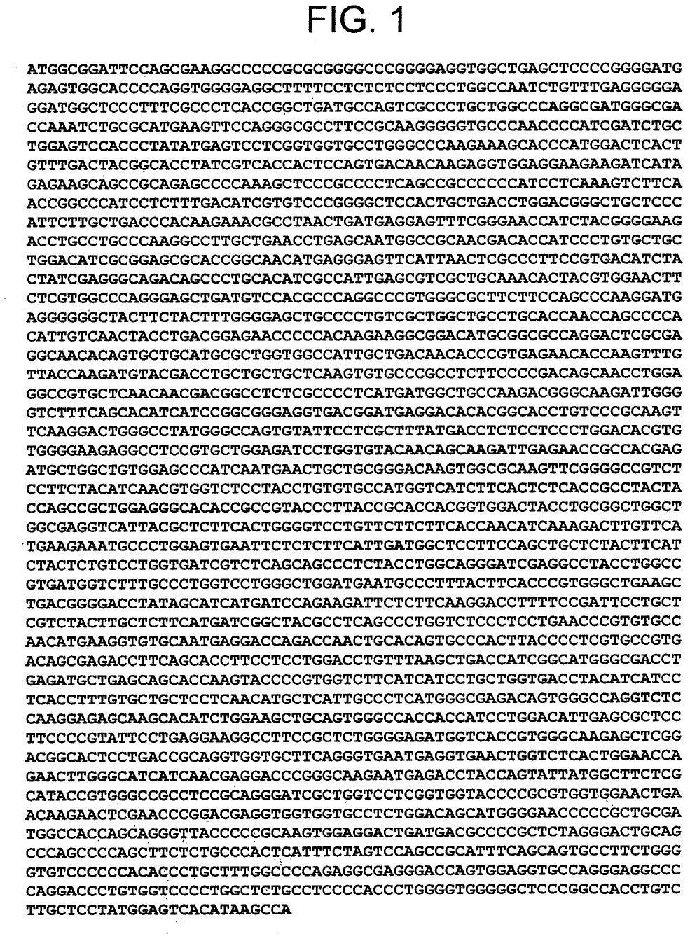

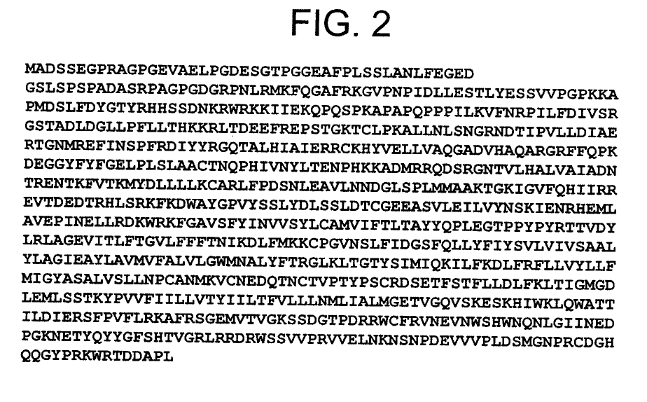

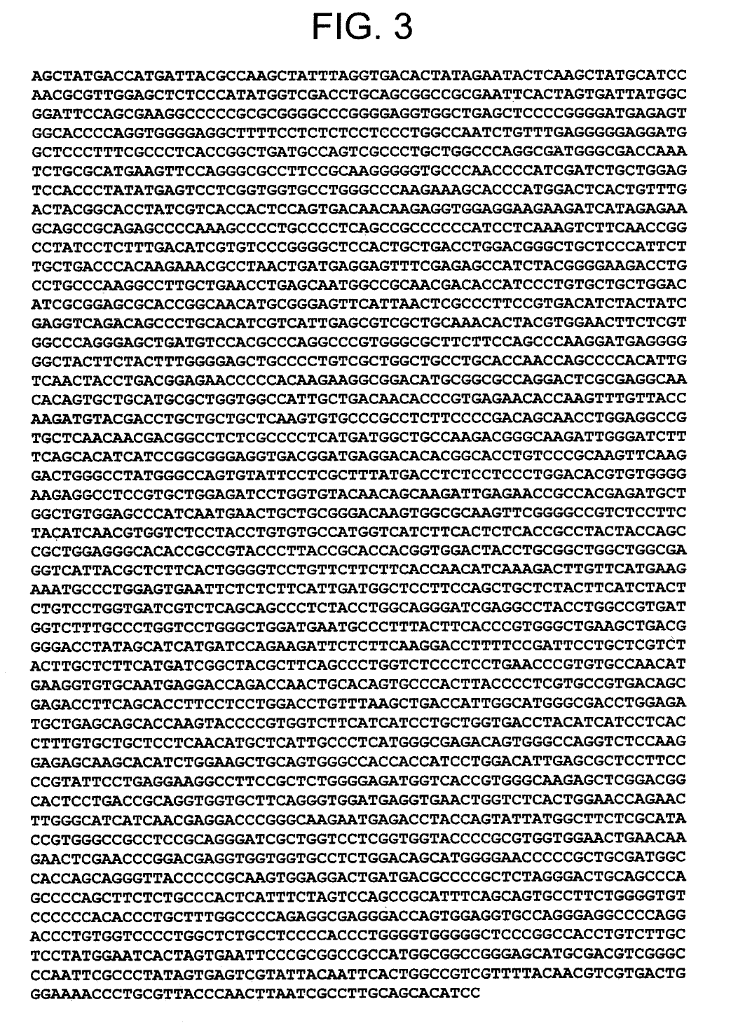

The invention also extends to the identification of materials that function as modulators of VR-OAC activity. In particular, the invention concerns the isolation, purification, and sequencing of certain nucleic acids that may correspond either in structure or function to the Vroac gene or its coding region in both mice and humans, as well as the corresponding polypeptides expressed by these nucleic acids. The invention thus comprises the discovery of nucleic acids having the nucleotide sequences set forth in FIGS. 1 and 3, and to degenerate variants, alleles and fragments thereof, all possessing the activity of sensing systemic osmotic pressure and/or mechanical stimuli of relevant organs and tissues. The invention extends to the proteins expressed by the nucleic acids of the invention, and particularly to those proteins set forth in FIGS. 2, 4 and 5, as well as to conserved variants, mutants and active fragments.

Various forms of VR-OAC, which may act as agonists or antagonists, may be prepared in pharmaceutical compositions, with a suitable carrier and at a strength effective for administration by various means to a patient experiencing abnormalities in osmoregulation or mechanoreception, which may be manifested in conditions such as hearing disorders; vertigo of labyrinthine origin; Meniere disease; arterial hypertension; kidney diseases characterized by disorders of fluid dysregulation and osmotic regulation; CNS disorders characterized by fluid dysregulation; neurological disorders, including e.g. ataxia due to alterations of afferent input to the CNS, paraesthesia, pain syndromes, memory impairment, Alzheimer's disease and other dementias, disorders of cerebrospinal fluid circulation, hydrocephalus; male infertility; immune dysfunction with alterations of antigen presentation, including HIV; obesity and diabetes mellitus; chronic obstructive lung disorder; bronchial asthma; sexual dysfunction due to sensory deficits; eye diseases, such as blindness due to corneal or retinal debilitiations or dysfunctions, glaucoma, orbital disease such as thyroid orbitopathy; and skin disorders, such as psoriasis, pemphigus vulgaris, pruritus, allergic skin diseases, alopecia and other forms of hair loss, baldness and excessive skin wrinkling; all alone or as part of an adverse medical condition such as cancer or AIDS, for the treatment thereof. A variety of administrative techniques may be utilized, among them oral administration, nasal and other forms of transmucosal administration, parenteral techniques such as subcutaneous, intravenous and intraperitoneal injections, catheterizations and the like. Appropriate quantities of VR-OAC, or analogs, small molecules mimics or antagonists thereof, as appropriate, may be so administered, in amounts that will vary and in particular, that should be based upon the recommendations and prescription of a qualified physician or veterinarian.

In accordance with the above, an assay system for screening potential drugs effective to mimic or antagonize the activity of VR-OAC may be prepared. The prospective drug may be contacted with a soluble form of VR-OAC, or alternatively may be used with cells that express a receptor form of VR-OAC, to determine whether it binds to, or activates (or antagonizes) VR-OAC. For example, in an expression assay system, the culture may be examined to observe any changes in the activity of the cells, due either to the addition of the prospective drug alone, or due to the effect of added quantities of the known osmotic pressure/mechanoresponsive activity modulator VR-OAC.

As stated earlier, the molecular cloning of the VR-OAC gene described herein has led to the identification of a class of materials that function on the molecular level to modulate osmotic and/or mechanical activity and responsiveness of cells, tissues and organs. The discovery of the modulators of the invention has important implications for the diagnosis and treatment of the disorders listed above. In addition, there are potential agricultural uses for the gene product in cases where one might wish to modulate the corresponding systems of animals. The discussion that follows with specific reference to the VR-OAC gene bears general applicability to the class of modulators that comprise a part of the present invention, and is therefore to be accorded such latitude and scope of interpretation.

In a particular embodiment, the functional activity of the VR-OAC polypeptide can be evaluated transgenically. The VR-OAC gene can be used in complementation studies employing transgenic mice. Transgenic vectors, including viral vectors, or cosmid clones (or phage clones) corresponding to the wild type locus of candidate gene, can be constructed using the isolated VR-OAC gene. Cosmids may be introduced into transgenic mice using published procedures [Jaenisch, Science, 240:1468-1474 (1988)].

The invention thereby includes transgenic animals wherein the expression of VR-OAC is altered, controlled or tagged with an identifier or marker. The invention thus encompasses transgenic animals wherein expression of VR-OAC is enhanced or blocked, so termed VR-OAC overexpressors and VR-OAC knockout animals. Also contemplated are transgenic animals wherein VR-OAC expression is under the control of a promoter or enhancer which may be selectively activated or inhibited. Further contemplated are transgenic animals wherein VR-OAC expression can be monitored or assayed by virtue of an epitope-tagged molecule or marker molecule, which is expressed from the VR-OAC promoter or as a VR-OAC-marker/tag fusion product.

Alternatively, VR-OAC genes can be tested by examining their phenotypic effects when expressed in antisense orientation in wild-type animals. In this approach, expression of the wild-type allele is suppressed, which leads to a mutant phenotype. RNA-RNA duplex formation (antisense-sense) prevents normal handling of mRNA, resulting in partial or complete elimination of wild-type gene effect. This technique has been used to inhibit TK synthesis in tissue culture and to produce phenotypes of the Kruppel mutation in Drosophila, and the Shiverer mutation in mice [Izant et al., Cell, 36:1007-1015 (1984); Green et al., Annu. Rev. Biochem., 55:569-597 (1986); Katsuki et al., Science, 241:593-595 (1988)]. An important advantage of this approach is that only a small portion of the gene need be expressed for effective inhibition of expression of the entire cognate mRNA. The antisense transgene will be placed under control of its own promoter or another promoter expressed in the correct cell type, and placed upstream of the SV40 polyA site. This transgene can be used to make transgenic mice.

In the long term, the VR-OAC gene product (the VR-OAC polypeptide or protein) is useful for identifying small molecule agonists and antagonists that affect its activity.

Various terms used throughout this specification shall have the definitions set out herein, for example, below.

The terms “VR-OAC”, “osmoreceptor”, “osmotic pressure modulator”, “mechanoreceptor”, “mechanoreception modulator”, “modulators”, and any variants not specifically listed, may be used herein interchangeably, and as used throughout the present application and claims refers in one instance to both nucleotides and to proteinaceous material, the latter including both single or multiple proteins. More specifically, the aforementioned terms extend to the nucleotides and to the DNA having the sequences described herein and presented in FIGS. 1 and 3 (SEQ ID NOS: 1 and 3). Likewise, the proteins having the amino acid sequence data described herein and presented in FIGS. 2, 4 and 5 are likewise contemplated, and in any of SEQ ID NOS: 2, 4, 5, 6, 7, 8, and 9, as are the profile of activities set forth with respect to all materials both herein and in the claims.

Additionally, nucleotides displaying substantially equivalent or altered activity are likewise contemplated, including substantially similar analogs and allelic variations. Likewise, proteins displaying substantially equivalent or altered activity, including proteins modified deliberately, as for example, by site-directed mutagenesis, or accidentally through mutations in hosts that produce the modulators are likewise contemplated.

The term “allelic variants” refers to the corresponding gene in different individuals that may have point mutations. For example, the various Vroac mutations represent allelic variants of VR-OAC.

The term “homologues” or “homologs”, in all of its grammatical forms, specifically includes the corresponding gene or protein from another species. In a specific embodiment, a homolog of murine VR-OAC is human VR-OAC. The term can also include genes or proteins mutated or altered, e.g., by substitution of variant amino acid residues from one species in the polypeptide of another, so as to correspond to an analogous gene or protein as if from another species. As is well known in the art, homologous genes can readily be identified by sequence similarity, hybridization with probes specific for the gene in another species, detection by PCR analysis using primers for a different species, or mapping to a syntenic location of the chromosome, to mention but a few such methods. Protein homology can be detected by antibody cross reactivity, similar protease digestion profile, comparable molecular weight and isoelectric points, and similar secondary or tertiary structure as evaluated by “in silico” amino acid alignment to mention some of the well known tests for homologous proteins.

The term “substantially similar” as used herein with respect to nucleic acid or amino acid sequences means at least 50% sequence similarity, preferably at least 60% sequence similarity, more preferably at least 70% sequence similarity, even more preferably at least 80% sequence similarity, and most preferably at least 90% sequence similarity.

The term “gene” as used herein refers to a nucleic acid, such as DNA, which codes on expression for a protein. Unless stated otherwise, gene may include mRNA, cDNA, or genomic DNA.

A composition comprising “A” (where “A” is a single protein, DNA molecule, vector, recombinant host cell, etc.) is substantially free of “B” (where “B” comprises one or more contaminating proteins, DNA molecules, vectors, etc., but excluding racemic forms of A) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

A “BAC” is a bacterial artificial chromosome; “STS” refers to sequence tagged site; a “YAC” is a yeast artificial chromosome. Other terms have the standard meanings ordinarily intended in the art.

### The VR-OAC Polypeptides

The terms “protein,” which refers to the naturally occurring polypeptide, and “polypeptide” are used herein interchangeably with respect to the VR-OAC gene product and variants thereof, which would include e.g., any homologs or orthologs thereof and any splice forms of the VR-OAC gene product.

As noted above, in specific embodiments polypeptides of the invention include those having the amino acid sequences set forth herein e.g., FIGS. 2, 4 and 5 and in any of SEQ ID NOS: 2, 4, 5, 6, 7, 8, and 9. The term further includes polypeptides modified with conservative amino acid substitutions, mutants, as well as biologically active fragments, analogs, and derivatives thereof. In yet another embodiment, the term includes polypeptides in which one or more cysteine residues or cystine pairs are replaced with serine, or a similar polar or neutral amino acid residue such as, but not necessarily limited to, threonine, methionine, or alanine.

The term “biologically active,” is used herein to refer to a specific effect of the polypeptide, including but not limited to specific binding, e.g., to VR-OAC, an anti-VR-OAC antibody, or other recognition molecule; activation of signal transduction pathways on a molecular level; and/or induction (or inhibition by antagonists) of physiological effects mediated by the native VR-OAC in vivo. VROAC polypeptides, including fragments, analogs, and derivatives, can be prepared synthetically, e.g., using the well known techniques of solid phase or solution phase peptide synthesis. Preferably, solid phase synthetic techniques are employed. Alternatively, VR-OAC polypeptides of the invention can be prepared using well known genetic engineering techniques, as described infra.

The structure of the VR-OAC polypeptide, preferably human VR-OAC polypeptide, can be analyzed by various methods known in the art. The protein sequence can be characterized by a hydrophilicity analysis [e.g., Hopp et al., Proc. Natl. Acad. Sci. USA, 78:3824 (1981)]. A hydrophilicity profile can be used to identify the hydrophobic and hydrophilic regions of the VR-OAC polypeptide, which may indicate regions buried in the interior of the folded polypeptide, the transmembrane domain, and regions accessible on the exterior of the polypeptide. In addition, secondary structural analysis [e.g., Chou et al, Biochem., 13:222 (1974)] can also be done, to identify regions of VR-OAC polypeptide that assume specific secondary structures. Manipulation of the predicted or determined structure, including secondary structure prediction, can be accomplished using computer software programs available in the art.

By providing an abundant source of recombinant VR-OAC polypeptide, the present invention enables quantitative structural determination of the polypeptide. In particular, enough material is provided for nuclear magnetic resonance (NMR), infrared (IR), Raman, and ultraviolet (UV), especially circular dichroism (CD), spectroscopic analysis. In particular NMR provides very powerful structural analysis of molecules in solution, which more closely approximates their native environment [Marion et al., Biochim. Biophys. Res. Comm., 113:967-974 (1983); Bar et al., J. Magn. Reson., 65:355-360 (1985); Kimura et al., Proc. Natl. Acad. Sci. USA, 77:1681-1685 (1980)]. Other methods of structural analysis can also be employed. These include but are not limited to X-ray crystallography [Engstom, Biochem. Exp. Biol., 11:7-13 (1974)]. In a preferred aspect, either soluble form or a membrane-binding form of VR-OAC is co-crystallized with VR-OAC to provide structural information about both molecules. In a more preferred embodiment, VROAC or soluble VR-OAC lacking one or more cystine crosslinks is used to form crystals or co-crystals with VR-OAC.

In yet a further embodiment, an analog of VR-OAC polypeptide can be tested to determine whether it cross-reacts with an antibody specific for native VR-OAC polypeptide, or specific fragments thereof. The degree of cross-reactivity provides information about structural homology or similarity of proteins, or about the accessibility of regions corresponding to portions of the polypeptide that were used to generate fragment-specific antibodies.

### Fragments of the VR-OAC Polypeptide

In a particular embodiment, the present invention contemplates that naturally occurring fragments, or truncated forms, of the VR-OAC polypeptide may be important. In addition to the naturally occurring isoforms of the polypeptide, the present invention further envisions recombinantly modified isoforms, e.g, by deletion of one or more of the cytoplasmic domain; the transmembrane domain; the ligand binding domain; the extracytoplasmic domain; or portions thereof. In particular VR-OAC fragments are described herein comprising the amino acid sequence set out in any of SEQ ID NOS: 5, 6 and 7.

### VR-OAC Polypeptide Chimeras

One or more of the splice-forms of the cytoplasmic domain can be used in a chimeric construct with another ligand-binding domain to artificially signal VROAC binding [e.g., Capon et al., U.S. Pat. No. 5,359,046, issued Oct. 25, 1994; Sanchez et al., J. Exp. Med., 178:1049 (1993); Burkhardt et al., Mol. Cell. Biol., 14:1095; International Patent Publications WO 96/23814, WO 96/23881, and WO 96/24671; Kotenko et al., J. Biol. Chem. 271:17174 (1996)]. In another embodiment, the extracytoplasmic (VR-OAC-binding) domain can be joined to a different cytoplasmic signal transduction domain, or alternatively to a glycosyl-phosphalidylinositol linker domain to provide for activation of cells via gp130.

### Analogs of the VR-OAC Polypeptide

The present invention specifically contemplates preparation of analogs of the VROAC polypeptide, which are characterized by being capable of a biological activity of VR-OAC polypeptide, or by binding to an anti-VR-OAC antibody. In one embodiment, the analog agonizes VR-OAC activity. Preferably, an VR-OAC agonist is more effective than the native protein. Such an analog may be particularly desirable for gene therapy, where increased signal transduction efficiency can compensate for any deficiency in the level of receptor expression. In another embodiment, the analog antagonizes VR-OAC activity.

In one embodiment, an analog of VR-OAC polypeptide is the VR-OAC polypeptide modified by substitution of amino acids at positions on the polypeptide that are not essential for structure or function. In a particular embodiment analogs or mutants of VR-OAC polypeptide are described herein, particularly VR-OAC polypeptides comprising the amino acid sequence set out in any of SEQ ID NOS: 8 and 9. For example, since it is expected and herein demonstrated that human VR-OAC polypeptide is biologically active in other species, substitution of divergent amino acid residues in the human sequence as compared to the amino acid sequence of the other specie will likely yield useful analogs of VR-OAC polypeptide.

Also contemplated by the present invention are analogs comprising conservative amino acid substitutions. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. In some instances, one polar amino acid may be substituted with another to preserve local hydrophilicity; more likely, a substitution that conserves charge, or at least does not introduce the opposite charge, is required. Such alterations will not be expected to affect apparent molecular weight as determined by polyacrylamide gel electrophoresis, or isoelectric point.

In still another embodiment, amino acid residues can be substituted with residues to form analogs of VR-OAC polypeptide that demonstrate enhanced propensity for forming, or which form more stable, secondary structures. For example, α-helix structure would be preferred if Glu, Ala, Leu, H is, Trp are introduced as substitutes for amino acid residues found in the ligand or other binding partner of VR-OAC. Preferably, conservative amino acid substitutions are employed, e.g., substituting aspartic acid with glutamic acid(s) (Glu); substituting isoleucine(s) with leucine; substituting glycine or valine, or any divergent amino acid (i.e., an amino acid that is not conserved between VR-OAC from different species), with alanine (e.g., serine at position 273 of the human VR-OAC polypeptide with alanine); substituting arginine or lysine with histidine; and substituting tyrosine and/or phenylalanine with tryptophan. Increasing the degree, or more importantly, the stability of α-helix structure may yield an VR-OAC analog with greater activity, increased binding affinity, or longer half-life. Also contemplated are truncated VR-OAC polypeptide analogs that incorporate structure-forming, e.g., helix-forming, amino acid residues to compensate for the greater propensity of polypeptide fragments to lack stable structure.

In another embodiment, an analog of the VR-OAC polypeptide, preferably the human VR-OAC polypeptide, is a truncated form of the polypeptide. For example, the preparation of a truncated form would be possible if it is determined e.g., that a particular domain or region of the molecule is not essential. In addition, the invention contemplates providing an VR-OAC analog having the minimum amino acid sequence necessary for a biological activity. This can be readily determined, e.g., by testing the activity of fragments of VR-OAC for the ability to bind to VROAC-specific antibodies, inhibit the activity of the native VR-OAC (by competitive binding), or agonize the activity of native VR-OAC.

It will be appreciated by one of ordinary skill in the art that the foregoing fragment sizes are approximate, and that additional amino acids e.g. from one to about five, can be included or deleted from each or both ends, or from the interior of the polypeptide or fragments thereof, of the recited truncated analogs.

Analogs, such as fragments, may be produced, for example, by digestion of the VR-OAC, e.g., with trypsin, chymotrypsin, pepsin, papain, thrombolytic proteases, carboxypeptidase A, proteinase-K, etc. Other analogs, such as muteins, can be produced by standard site-directed mutagenesis of weight modulator peptide coding sequences.

### Derivatives of VR-OAC Polypeptides

Generally, the present polypeptide may be derivatized by the attachment of one or more chemical moieties to the polypeptide moiety. The chemically modified derivatives may be further formulated for intraarterial, intraperitoneal, intramuscular, subcutaneous, intravenous, oral, nasal, rectal, buccal, sublingual, pulmonary, topical, transdermal, or other routes of administration. Chemical modification of biologically active proteins has been found to provide additional advantages under certain circumstances, such as increasing the stability and circulation time of the therapeutic protein and decreasing immunogenicity [see U.S. Pat. No. 4,179,337, Davis et al., issued Dec. 18, 1979; for a review, see Abuchowski et al., “Soluble Polymer-Enzyme Adducts”, in Enzymes as Drugs, pp. 367-383, Holcenberg and Roberts, eds., Wiley-Interscience, New York, N.Y., (1981)]. A review article describing protein modification and fusion proteins is Francis, Focus on Growth Factors, 3:4-10 (1992).

### Chemical Moieties For Derivatization

The chemical moieties suitable for derivatization may be selected from among various polymers, in particular water soluble polymers. The polymer selected is preferably water soluble so that the protein to which it is attached does not precipitate in an aqueous environment, such as a physiological environment. However, apolar polymers can also be used where a particular application benefits from their use, e.g., in a controlled release matrix in which accessibility of water is restricted. Preferably, for therapeutic use of the end-product preparation, the polymer will be pharmaceutically acceptable. One skilled in the art will be able to select the desired polymer based on such considerations as whether the polymer/protein conjugate will be used therapeutically, and if so, the desired dosage, circulation time, resistance to proteolysis, and other considerations. For the present proteins and peptides, these may be ascertained using the assays provided herein.

### Polymer Molecules

The water soluble polymer may be selected from the group consisting of, for example, polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, olyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols and polyvinyl alcohol. Polyethylene glycol propionaldenhyde may provide advantages in manufacturing due to its stability in water.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

### Polymer/Protein Ratio

The number of polypeptide molecules attached to each polymer may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to protein (or peptide) molecules will vary, as will their concentrations in the reaction mixture. In general, the optimum ratio (in terms of efficiency of reaction in that there is no excess unreacted protein or polymer) will be determined by factors such as the desired degree of derivatization (e.g., mono, di-, tri-, etc.), the molecular weight of the polymer selected, whether the polymer is branched or unbranched, and the reaction conditions.

### Attachment of the Chemical Moiety to the Protein

The polyethylene glycol molecules (or other chemical moieties) should be attached to the protein with consideration of effects on functional or antigenic domains of the protein. There are a number of attachment methods available to those skilled in the art, e.g., EP 0 401 384 herein incorporated by reference (coupling PEG to G-CSF). See also Malik et al., Exp. Hematol., 20:1028-1035 (1992) (reporting pegylation of GM-CSF using tresyl chloride). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecule(s). Preferred for therapeutic purposes is attachment at an amino group, such as attachment at the N-terminus or lysine group. Attachment at residues important for receptor binding should be avoided if receptor binding is desired.

### N-terminally Chemically Modified Proteins.

One may specifically desire N-terminally chemically modified protein. Using polyethylene glycol as an illustration of the present compositions, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemical modification may be accomplished by reductive alkylation which exploits differential reactivity of different types of primary amino groups (lysine versus the N-terminus) available for derivatization in a particular protein. Under the appropriate reaction conditions, substantially selective derivatization of the protein at the N-terminus with a carbonyl group containing polymer is achieved. For example, one may selectively N-terminally pegylate the protein by performing the reaction at a pH which allows one to take advantage of the pKₐ differences between the E-amino groups of the lysine residues and that of the α-amino group of the N-terminal residue of the protein. By such selective derivatization attachment of a water soluble polymer to a protein is controlled: the conjugation with the polymer takes place predominantly at the N-terminus of the protein and no significant modification of other reactive groups, such as the lysine side chain amino groups, occurs. Using reductive alkylation, the water soluble polymer may be of the type described above, and should have a single reactive aldehyde for coupling to the protein. Polyethylene glycol propionaldehyde, containing a single reactive aldehyde, may be used.

### Nucleic Acids Associated With VR-OAC Polypeptide

As noted above, the present invention is directed to nucleic acids encoding VROAC polypeptides, as well as associated genomic non-coding sequences 5′, 3′, and intronic to the VR-OAC gene. Thus, in accordance with the present invention there may be employed conventional molecular biology, microbiology, and recombinant DNA techniques within the skill of the art. Such techniques are explained fully in the literature [see, e.g., Sambrook et al., Molecular Cloning: A Laboratory Manual, Second Edition, Cold Spring Harbor Laboratory Press, Cold Spring Harbor, N.Y. (1989); Glover ed., DNA Cloning: A Practical Approach, Volumes I and II, MRL Press, Ltd., Oxford, U.K. (1985); Gait ed., Oligonucleotide Synthesis, Oxford University Press (1984); Hames et al., eds., Nucleic Acid Hybridization, Springer-Verlag (1985); Hames et al., eds. Transcription And Translation, Oxford University Press (1984); Freshney ed., Animal Cell Culture, Oxford University Press (1986); Immobilized Cells And Enzymes, IRL Press (1986); Perbal, A Practical Guide To Molecular Cloning, Wiley, New York (1984)]. Of particular relevance to the present invention are strategies for isolating, cloning, sequencing, analyzing, and characterizing a gene or nucleic acid based on the well known polymerase chain reaction (PCR) techniques.

A “replicon” is any genetic element (e.g., plasmid, chromosome, virus) that functions as an autonomous unit of DNA replication in vivo, i.e., capable of replication under its own control.

A “vector” is a replicon, such as a plasmid, phage or cosmid, to which another DNA segment may be attached so as to bring about the replication of the attached segment.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

“Heterologous” DNA refers to DNA not naturally located in the cell, or in a chromosomal site of the cell. Preferably, the heterologous DNA includes a gene foreign to the cell.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

A “clone” is a population of cells derived from a single cell or common ancestor by mitosis.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single-stranded form, or a double-stranded helix. Double-stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary or quaternary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single-stranded form of the nucleic acid molecule can anneal to the other nucleic acid molecule under the appropriate conditions of temperature and solution ionic strength (see Sambrook et al., 1989, supra). The conditions of temperature and ionic strength determine the “stringency” of the hybridization. For preliminary screening for homologous nucleic acids, low stringency hybridization conditions, corresponding to a Tₘ of 55° C., can be used, e.g., 5× SSC, 0.1% SDS, 0.25% milk, and no formamide; or 30% formamide, 5× SSC, 0.5% SDS). Moderate stringency hybridization conditions correspond to a higher Tₘ, e.g., 40% formamide, with 5× or 6× SCC. High stringency hybridization conditions correspond to the highest Tₘ, e.g., 50% formamide, 5× or 6× SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., 1989, supra, 9.50-0.51). For hybridization with shorter nucleic acids, i.e., oligonucleotides, the position of mismatches becomes more important, and the length of the oligonucleotide determines its specificity (see Sambrook et al., 1989, supra, 11.7-11.8). Preferably a minimum length for a hybridizable nucleic acid is at least about 10 nucleotides; more preferably at least about 15 nucleotides; most preferably the length is at least about 20 nucleotides.

In a specific embodiment, the term “standard hybridization conditions” refers to a Tₘ of 55° C., using conditions as set forth above. In a preferred embodiment, the Tₘ is 60° C.; in a more preferred embodiment, the Tₘ is 60° C.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

### Isolation of VR-OAC Coding and Flanking Sequences

The nucleic acids contemplated by the present invention include nucleic acids that code on expression for peptides such as those set forth in FIGS. 2, 4 and 5 and set out in any of SEQ ID NOS: 2, 4, 5, 6, 7, 8, and 9. Accordingly, while specific DNA has been isolated and sequenced in relation to the VR-OAC gene, any animal cell potentially can serve as the nucleic acid source for the molecular cloning of a gene encoding the polypeptides of the invention. The DNA may be obtained by standard procedures known in the art from cloned DNA (e.g., a DNA “library”), by chemical synthesis, by cDNA cloning, or by the cloning of genomic DNA, or fragments thereof, purified from the desired cell [see, for example, Sambrook et al., 1989, supra; Glover, 1985, supra]. Clones derived from genomic DNA may contain regulatory and intronic DNA regions in addition to coding regions; clones derived from cDNA will not contain intron sequences. Whatever the source, the gene should be molecularly cloned into a suitable vector for propagation of the gene.

In the molecular cloning of the gene from genomic DNA, the genomic DNA can be amplified using primers selected from the cDNA sequences. Alternatively, DNA fragments are generated, some of which will encode the desired gene. The DNA may be cleaved at specific sites using various restriction enzymes. One may also use DNase in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired VR-OAC-gene may be accomplished in a number of ways. For example, if an amount of a portion of a VR-OAC-gene or its specific RNA, or a fragment thereof, is available and can be purified and labeled, the generated DNA fragments may be screened by nucleic acid hybridization to a labeled probe [Benton et al., Science, 196:180 (1977); Grunstein et al., Proc. Natl. Acad. Sci. USA, 72:3961 (1975)]. The present invention provides such nucleic acid probes, which can be conveniently prepared from the specific sequences disclosed herein, e.g., a hybridizable probe having a nucleotide sequence corresponding to at least a 10, preferably a 15, and more preferably at least a 20 nucleotide fragment of the sequences depicted in FIGS. 1 and 3, and set out in SEQ ID NOS: 2 and 4, as well as SEQ ID NOS: 5, 6, 7, 8, and 9. Preferably, a fragment is selected that is highly unique to the nucleic acids of the invention. Those DNA fragments with substantial sequence similarity to the probe, e.g., a homologous DNA, will hybridize. As noted above, the greater the degree of sequence similarity, the more stringent the hybridization conditions that can be used. In one embodiment, low stringency hybridization conditions are used to identify a homologous VR-OAC receptor nucleic acid. However, in a preferred aspect, and as demonstrated experimentally herein, a nucleic acid encoding a polypeptide of the invention will hybridize to a nucleic acid having a nucleotide sequence such as depicted in FIGS. 1 and 3, or a hybridizable fragment thereof, under moderately stringent conditions; more preferably, it will hybridize under high stringency conditions.

In another specific embodiment, the DNA of the invention can be identified using one of the PCR probes obtained by exon trapping and cDNA selection. For example, probes such as are described in Example 1 can be used to will amplify a DNA of the invention.

Preferably, these primers will amplify DNA under moderately to high stringency conditions, e.g., using pre-hybridization at 65° using Rapid-hyb buffer (Amersham Life Sciences), followed by hybridization for 6 hours at 65°, followed by washing first with 2× SSC/0.1% SDS for 30 min at room temperature (RT), and a second wash at higher stringency with 0.3× SSC/0.1% SDS, RT, for 30 min. As will be appreciated by those of skill in the art, the stringency of the second wash is flexible and depends on the length of the probe and the degree of sequence similarity of each probe. For example, since human and mouse coding regions are highly homologous, the same hybridization conditions may be employed with a lower the stringency second wash (e.g., twice with 2× SSC/0.1% SDS, RT). If this results in no signal with no-background, hybridization can be attempted at a lower temperature (lower stringency), e.g., 42° C. If there is too much background, the stringency of the second wash can be increased, (e.g., 0.5 or 0.3× SSC, 0.1% SDS, RT). According to the invention, the above-noted PCR probes will define a nucleic acid molecule, e.g., DNA, encoding VR-OAC from human as well as murine DNA libraries under similar hybridization conditions.

Alternatively, the presence of the gene may be detected by assays based on the physical, chemical, or immunological properties of its expressed product. For example, cDNA clones, or DNA clones which hybrid-select the proper mRNAs, can be selected which produce a protein that, e.g., has similar or identical electrophoretic migration, isoelectric focusing behavior, proteolytic digestion maps, VR-OAC binding activity, or antigenic properties as known for the present VROAC. For example, antibodies of the instant invention can conveniently be used to screen for homologs of VR-OAC from other sources. Preferably, proteins from candidate genes are tested for VR-OAC binding.

A gene encoding a polypeptide of the invention can also be identified by mRNA selection, i.e., by nucleic acid hybridization followed by in vitro translation. In this procedure, fragments are used to isolate complementary mRNAs by hybridization.

Such DNA fragments may represent available, purified modulator DNA. Immunoprecipitation analysis or functional assays (e.g., VR-OAC binding activity) of the in vitro translation products of the products of the isolated mRNAs identifies the mRNA and, therefore, the complementary DNA fragments, that contain the desired sequences. In addition, specific mRNAs may be selected by adsorption of polysomes isolated from cells to immobilized antibodies specifically directed against a VR-OAC peptide.

A radiolabeled VR-OAC peptide cDNA can be synthesized using the selected mRNA (from the adsorbed polysomes) as a template. The radiolabeled mRNA or cDNA may then be used as a probe to identify homologous modulator peptide DNA fragments from among other genomic DNA fragments.

As mentioned above, a DNA sequence encoding weight modulator peptides as disclosed herein can be prepared synthetically rather than cloned. The DNA sequence can be designed with the appropriate codons for the VR-OAC amino acid sequences. In general, one will select preferred codons for the intended host if the sequence will be used for expression. The complete sequence may be assembled from overlapping oligonucleotides prepared by standard methods and assembled into a complete coding sequence [see, e.g., Edge, Nature, 292:756 (1981); Nambair et al., Science, 223:1299 (1984); Jay et al., J. Biol. Chem., 259:6311 (1984)].

Synthetic DNA sequences allow convenient construction of genes that will express VR-OAC analogs, as described above. Alternatively, DNA encoding analogs can be made by site-directed mutagenesis of native VR-OAC genes or cDNAs, and analogs can be made directly using conventional polypeptide synthesis.

A general method for site-specific incorporation of unnatural amino acids into proteins is described in Noren et al, Science, 244:182-188 (1989). This method may be used to create analogs of the VR-OAC polypeptide with unnatural amino acids.

Due to the degeneracy of nucleotide coding sequences, other DNA sequences which encode substantially the same amino acid sequence as a VR-OAC gene may be used in the practice of the present invention. These include but are not limited to allelic genes, homologous genes from other species, and nucleotide sequences comprising all or portions of VR-OAC genes which are altered by the substitution of different codons that encode the same amino acid residue within the sequence, thus producing a silent change. Likewise, the VR-OAC derivatives of the invention include, but are not limited to, those containing, as a primary amino acid sequence, all or part of the amino acid sequence of a VR-OAC protein including altered sequences in which functionally equivalent amino acid residues are substituted for residues within the sequence resulting in a conservative amino acid substitution, as described above in connection with VR-OAC analogs.

### Non-Coding Nucleic Acids

The present invention extends to the preparation of antisense nucleotides and ribozymes that may be used to interfere with the expression of the proteins at the translational level. This approach utilizes antisense nucleic acid and ribozymes to block translation of a specific mRNA, either by masking that mRNA with an antisense nucleic acid or cleaving it with a ribozyme.

Antisense nucleic acids are DNA or RNA molecules that are complementary to at least a portion of a specific mRNA molecule [See Weintraub, Sci. Am., 262:40-46 (1990); Marcus-Sekura, Anal. Biochem., 172:289-295 (1988)]. In the cell, they hybridize to that mRNA, forming a double-stranded molecule. The cell does not translate an mRNA complexed in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into weight modulator peptide-producing cells. Antisense methods have been used to inhibit the expression of many genes in vitro [(Marcus-Sekura, 1988 supra; Hambor et al., J. Exp. Med., 168:1237-1245 (1988)].

Ribozymes are RNA molecules possessing the ability to specifically cleave other single-stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it [Cech, J. Am. Med. Assoc., 260:3030-3034 (1988)]. Because ribozymes are sequence-specific, only mRNAs with particular sequences are inactivated.

Investigators have identified two types of ribozymes, Tetrahymena-type and “hammerhead”-type. Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The DNA sequences described herein may thus be used to prepare antisense molecules against and ribozymes that cleave mRNAs for osmo/mechanoreceptor modulator proteins and their ligands, thus inhibiting expression of the VR-OAC gene.

In another embodiment, short oligonucleotides complementary to the coding and complementary strands of the VR-OAC nucleic acid, or to non-coding regions of the VR-OAC gene 5′, 3′, or internal (intronic) to the coding region are provided by the present invention. Such nucleic acids are useful as probes, either as directly labeled oligonucleotide probes, or as primers for the polymerase chain reaction, for evaluating the presence of mutations in the VR-OAC gene, or the level of expression of VR-OAC mRNA. Preferably, the non-coding nucleic acids of the invention are from the human VR-OAC gene.

In a specific embodiment, the non-coding nucleic acids provide for homologous recombination for integration of an amplifiable gene and/or other regulatory sequences in proximity to the VR-OAC gene, e.g., to provide for higher levels of expression of the VR-OAC polypeptide, or to overcome a mutation in the VR-OAC gene regulatory sequences that prevent proper levels of expression of the VR-OAC polypeptide [See International Patent Publication WO 91/06666, published May 16, 1991 by Skoultchi; International Patent Publication No. WO 91/09955, published Jul. 11, 1991 by Chappel; see also International Patent Publication No. WO 90/14092, published Nov. 29, 1990, by Kucherlapati and Campbell].

### Production of VR-OAC Polypeptide: Expression and Synthesis

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A coding sequence is “under the control” of transcriptional and translational control sequences in a cell when RNA polymerase transcribes the coding sequence into mRNA, which is then trans-RNA spliced and translated into the protein encoded by the coding sequence.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted upstream (5′) of and in reading frame with the gene.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal, and synthetic DNA sequences. Suitable vectors include derivatives of SV40 and known bacterial plasmids, e.g., E. coli plasmids col E1, pCR1, pBR322, pMB9, pUC or pUC plasmid derivatives, e.g., pGEX vectors, pET vectors, pmal-c, pFLAG, etc., and their derivatives, plasmids such as RP4; phage DNAs, e.g., the numerous derivatives of phage λ, such as NM989, and other phage DNA, e.g., M13 and filamentous single-stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the AOX 1 promoter of methylotrophic yeast, the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces; fungi such as yeasts (Saccharomyces, and methylotrophic yeast such as Pichia, Candida, Hansenula, and Torulopsis); and animal cells, such as CHO, R1.1, B-W and LM cells, African Green Monkey kidney cells (e.g., COS 1, COS 7, BSC1, BSC40, and BMT10), insect cells (e.g., Sf9), and human cells and plant cells in tissue culture. Particularly preferred is expression in baculovirus with an insect signal peptide replacing the VR-OAC signal peptide, for example, in vector pMelBac (Invitrogen; Catalog No. V1950-20).

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors, a person skilled in the art will be able to construct a variety of vector/expression control sequence/host combinations that will express the DNA sequences of this invention on fermentation or in large scale animal culture.

In a specific embodiment, an VR-OAC fusion protein can be expressed. An VROAC fusion protein comprises at least a functionally active portion of a non-VROAC protein joined via a peptide bond to at least a functionally active portion of a VR-OAC binding partner. The non-VR-OAC sequences can be amino- or carboxy-terminal to the VR-OAC sequences. For example, in preparing “artificial” receptors, joining the VR-OAC encoding coding domain for the VR-OAC binding portion at the 5′ position will yield a protein that binds VR-OAC and mediates some other action based on the non-VR-OAC protein's activity. Conversely, joining a different protein (such as a growth factor, cytokine, or hormone receptor binding coding domain) 5′ to a VR-OAC cytoplasmic coding domain will allow for activation via VR-OAC upon binding a different ligand than VR-OAC. In another embodiment, a chimeric construct may simply facilitate expression of VR-OAC. In a specific embodiment, infra, VR-OAC and fragments thereof are expressed with an N-terminal melittin signal peptide.

In another aspect, the pGEX vector [Smith et al., Gene 67:31-40 (1988)] can be used. This vector fuses the schistosoma japonicum glutathionine S-transferase cDNA to the sequence of interest. Bacterial proteins are harvested and recombinant proteins can be quickly purified on a reduced glutathione affinity column. The GST carrier can subsequently be cleaved from fusion proteins by digestion with site-specific proteases. After cleavage, the carrier and uncleaved fusion protein can be removed by absorption on glutathione agarose. Difficulty with the system occasionally arises when the encoded protein is insoluble in aqueous solutions.

Expression of recombinant proteins in bacterial systems may result in incorrect folding of the expressed protein, requiring refolding. The recombinant protein can be refolded prior to or after cleavage to form a functionally active polypeptide. The polypeptide may be refolded by the steps of (i) incubating the protein in a denaturing buffer that contains a reducing agent, and then (ii) incubating the protein in a buffer that contains an oxidizing agent, and preferably also contains a protein stabilizing agent or a chaotropic agent, or both. Suitable redox (reducing/oxidizing) agent pairs include, but are not limited to, reduced glutathione/glutathione disulfide, cystine/cysteine, cystamine/cysteamine, and 2-mercaptoethanol/2hydroxyethyldisulfide. In a particular aspect, the fusion protein can be solubilized in a denaturant, such as urea, prior to exchange into the reducing buffer. In preferred embodiment, the protein is also purified, e.g., by ion exchange or Ni-chelation chromatography, prior to exchange into the reducing buffer. Denaturing agents include but are not limited to urea and guanidine-HCl. The recombinant protein is then diluted about at least 10-fold, more preferably about 100-fold, into an oxidizing buffer that contains an oxidizing agent, such as but not limited to 0.1 M Tris-HCl, pH 8.0, 1 mM EDTA, 0.15 M NaCl, 0.3 M oxidized glutathione. The fusion protein is then incubated for about 1 to about 24 hours, preferably about 2 to about 16 hours, at room temperature in the oxidizing buffer. The oxidizing buffer may comprise a protein stabilizing agent, e.g., a sugar, an alcohol, or ammonium sulfate. The oxidizing buffer may further comprise a chaotropic agent at low concentration, to destabilize incorrect intermolecular interactions and thus promote proper folding. Suitable chaotropic agents include but are not limited to a detergent, a polyol, L-arginine, guanidine-HCl and polyethylene glycol (PEG). It is important to use a low enough concentration of the chaotropic agent to avoid denaturing the protein. The refolded protein can be concentrated by at least about 10-fold, more preferably by the amount it was diluted into the oxidizing buffer.

Alternatively, the invention contemplates periplasmic expression of a protein of the invention.

Bacterial fermentation processes can also result in a recombinant protein preparation that contains unacceptable levels of endotoxins. Therefore, the invention contemplates removal of such endotoxins, e.g., by using endotoxin-specific antibodies or other endotoxin binding molecules. The presence of endotoxins can be determined by standard techniques, such as by employing E-TOXATE Reagents (Sigma, St. Louis, Mo.), or with bioassays.

In addition to the specific example, the present inventors contemplate use of baculovirus, mammalian, and yeast expression systems to express the VR-OAC protein. For example, in baculovirus expression systems, both non-fusion transfer vectors, such as but not limited to pVL941 (BamH1 cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedrin initiation codon; Invitrogen (195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220)). In a specific embodiment, infra, the pMel Bac expression vector (Invitrogen) is employed.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR [see Kaufman, Current Protocols in Molecular Biology, 16.12 (1991)]). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein Ia gene promoter, hygromycin selectable marker: Invitrogen), pREP8 (BamH1, XhoI, NotI, HindIII, NheI, and KpnI cloning site, RSV-LTR promoter, histidinol selectable marker, Invitrogen), pREP9 (KpnI, NheI, HindIII, NotI, XhoI, SfiI, and BamH1 cloning site, RSV-LTR promoter, G418 selectable marker; Invitrogen), and pEBVH is (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors [see, Kaufman, 1991, supra)] for use according to the invention include but are not limited to pSC11 (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF1S (EcoRI, PstI, SalI, AccI, HindIII, SbaI, BamH1, and Hpa cloning site, TK or XPRT selection).

Yeast expression systems can also be used according to the invention to express OB polypeptide. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

In addition to recombinant expression of VR-OAC polypeptide, the present invention envisions and fully enables preparation of VR-OAC polypeptide, or fragments thereof, using the well known and highly developed techniques of solid phase peptide synthesis. The invention contemplates using both the popular Boc and Fmoc, as well as other protecting group strategies, for preparing VR-OAC polypeptide or fragments thereof. Various techniques for refolding and oxidizing the cysteine side chains to form a disulfide bond are also well-known in the art.

### Antibodies to the VR-OAC Polypeptide

According to the invention, VR-OAC polypeptide produced recombinantly or by chemical synthesis, and fragments or other derivatives or analogs thereof, including fusion proteins, may be used as an immunogen to generate antibodies that recognize the VR-OAC polypeptide. Such antibodies include but are not limited to polyclonal, monoclonal, chimeric, single chain, Fab fragments, and an Fab expression library.

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10, amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567, as well as antigen binding portions of antibodies, including Fab, F(ab′)₂ and F(v) (including single chain antibodies). Accordingly, the phrase “antibody molecule” in its various grammatical forms as used herein contemplates both an intact immunoglobulin molecule and an immunologically active portion of an immunoglobulin molecule containing the antibody combining site. An “antibody combining site” is that structural portion of an antibody molecule comprised of heavy and light chain variable and hypervariable regions that specifically binds antigen.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)2 and F(v), which portions are preferred for use in the therapeutic methods described herein.

Fab and F(ab′)2 portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also wellknown and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The term “adjuvant” refers to a compound or mixture that enhances the immune response to an antigen. An adjuvant can serve as a tissue depot that slowly releases the antigen and also as a lymphoid system activator that non-specifically enhances the immune response [Hood et al., in Immunology, p. 384, Second Ed., Benjamin/Cummings, Menlo Park, Calif. (1984)]. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

Various procedures known in the art may be used for the production of polyclonal antibodies to VR-OAC polypeptide, or fragment, derivative or analog thereof. For the production of antibody, various host animals can be immunized by injection with the VR-OAC polypeptide, or a derivative (e.g., fragment or fusion protein) thereof, including but not limited to rabbits, mice, rats, sheep, goats, etc. In one embodiment, the VR-OAC polypeptide or fragment thereof can be conjugated to an immunogenic carrier, e.g., bovine serum albumin (BSA) or keyhole limpet hemocyanin (KLH). Specific VR-OAC antigenic fragments may be derived from FIGS. 2, 4 and 5. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the VR-OAC polypeptide, or fragment, analog, or derivative thereof, any technique that provides for the production of antibody molecules by continuous cell lines in culture may be used. These include but are not limited to the hybridoma technique originally developed by Kohler et al., Nature, 256:495-497 (1975), as well as the trioma technique, the human B-cell hybridoma technique [Kozbor et al., Immunology Today, 4:72 (1983)], and the EBV-hybridoma technique to produce human monoclonal antibodies [Cole et al., in Monoclonal Antibodies and Cancer Therapy, pp. 77-96, Alan R. Liss, Inc., (1985)]. Immortal, antibody-producing cell lines can be created by techniques other than fusion, such as direct transformation of B lymphocytes with oncogenic DNA, or transfection with Epstein-Barr virus [see, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; and 4,493,890].

In an additional embodiment of the invention, monoclonal antibodies can be produced in germ-free animals [International Patent Publication No. WO 89/12690, published Dec. 28, 1989]. According to the invention, human antibodies may be used and can be obtained by using human hybridomas [Cote et al., Proc. Natl. Acad. Sci. USA, 80:2026-2030 (1983)] or by transforming human B cells with EBV virus in vitro (Cole et al., 1985, supra). In fact, according to the invention, techniques developed for the production of “chimeric antibodies” [Morrison et al., J. Bacteriol., 159-870 (1984); Neuberger et al., Nature, 312:604-608 (1984); Takeda et al., Nature, 314:452-454 (1985)] by splicing the genes from a mouse antibody molecule specific for the VR-OAC polypeptide together with genes from a human antibody molecule of appropriate biological activity can be used; such antibodies are within the scope of this invention. Such human or humanized chimeric antibodies are preferred for use in therapy of human diseases or disorders (described herein) since the human or humanized antibodies are much less likely than xenogenic antibodies to induce an immune response, in particular an allergic response, themselves.

According to the invention, techniques described for the production of single chain antibodies (U.S. Pat. Nos. 5,476,786 and 5,132,405 to Huston; U.S. Pat. No. 4,946,778) can be adapted to produce VR-OAC polypeptide-specific single chain antibodies. An additional embodiment of the invention utilizes the techniques described for the construction of Fab expression libraries [Huse et al., Science, 246:1275-1281 (1989)] to allow rapid and easy identification of monoclonal Fab fragments with the desired specificity for an ob polypeptide, or its derivatives, or analogs.

Antibody fragments which contain the idiotype of the antibody molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a VR-OAC polypeptide, one may assay generated hybridomas for a product which binds to a VR-OAC polypeptide fragment containing such epitope. For selection of an antibody specific to a VR-OAC polypeptide from a particular species of animal, one can select on the basis of positive binding with VR-OAC polypeptide expressed by or isolated from cells of that species of animal.

The foregoing antibodies can be used in methods known in the art relating to the localization and activity of the VR-OAC polypeptide, e.g., for Western blotting, imaging VR-OAC polypeptide in situ, measuring levels thereof in appropriate physiological samples, detecting expression of VR-OAC, etc.

In a specific embodiment, antibodies that agonize or antagonize the activity of VROAC polypeptide can be generated. Such antibodies can be tested using the assays described infra for identifying ligands.

In a particular aspect, antibodies are developed by immunizing rabbits with synthetic peptides predicted by the protein sequence or with recombinant proteins made using bacterial expression vectors. The choice of synthetic peptides is made after careful analysis of the predicted protein structure, as described above. In particular, peptide sequences between putative cleavage sites are chosen. Synthetic peptides are conjugated to a carrier such as KLH hemocyanin or BSA using carbodiimide and used in Freunds adjuvant to immunize rabbits. In order to prepare recombinant protein, the pGEX vector can be used to express the polypeptide [Smith et al., 1988, supra]. Alternatively, one can use only hydrophilic domains to generate the fusion protein. The expressed protein will be prepared in quantity and used to immunize rabbits in Freunds adjuvant.

In a specific embodiment, infra, peptides corresponding to particular amino acid residues from the human VR-OAC polypeptide depicted in either of FIGS. 2, 4 or 5 can be generated by solid phase peptide synthesis or by expression, optionally conjugated to a carrier such as KLH, and used to immunize rabbits, rats, goats, chickens, etc.

In another specific embodiment, recombinant VR-OAC polypeptide is used to immunize chickens, and the chicken anti-VR-OAC antibodies are recovered from egg yolk, e.g., by affinity purification on an VR-OAC-column. Preferably, chickens used in immunization are kept under specific pathogen free (SPF) conditions.

In yet another embodiment, recombinant VR-OAC polypeptide is used to immunize rabbits, and the polyclonal antibodies are immunopurified prior to further use. The purified antibodies are particularly useful for semi-quantitative assays, particularly for detecting the presence of the circulating (soluble) splice form(s) of VR-OAC polypeptide in serum or plasma.

Panels of monoclonal antibodies produced against modulator peptides can be screened for various properties; i.e., isotype, epitope, affinity, etc. Of particular interest are monoclonal antibodies that neutralize the activity of the modulator peptides. Such monoclonals can be readily identified in activity assays for the osmo/mechanoreception modulators. High affinity antibodies are also useful when immunoaffinity purification of native or recombinant polypeptide is desired.

Preferably, the anti-modulator antibody used in the diagnostic and therapeutic methods of this invention is an affinity-purified polyclonal antibody. More preferably, the antibody is a monoclonal antibody (mAb). In addition, it is preferable for the anti-modulator antibody molecules used herein be in the form of Fab, Fab′, F(ab′)₂ or F(v) portions of whole antibody molecules.

### Diagnostics

The present invention also relates to a variety of diagnostic applications, including methods for detecting the presence of conditions and/or stimuli that impact upon abnormalities in sensory organ function as defined herein, by reference to their ability to elicit the activities which are mediated by the present VR-OAC polypeptides. As mentioned earlier, the peptides can be used to produce antibodies to themselves by a variety of known techniques, and such antibodies could then be isolated and utilized as in tests for the presence of particular transcriptional activity in suspect target cells. Alternatively, the nucleic acids of the invention can be employed in diagnosis.

### Antibody-Based Diagnostics

As suggested earlier, a diagnostic method useful in the present invention comprises examining a cellular sample or medium by means of an assay including an effective amount of an VR-OAC binding partner, such as an anti-modulator antibody or VROAC, preferably an affinity-purified polyclonal antibody, and more preferably a mAb. In addition, it is preferable for the antibody molecules used herein be in the form of Fab, Fab′, F(ab′)₂ or F(v) portions or whole antibody molecules. As previously discussed, patients capable of benefiting from this method include those suffering from conditions where abnormal osmotic pressure or mechanoreception is an element of the condition.

Also, antibodies including both polyclonal and monoclonal antibodies, may possess certain diagnostic applications and may for example, be utilized for the purpose of detecting and/or measuring conditions where abnormalities in osmotic pressure or mechanoreception are or may be likely to develop.

VR-OAC can be detected from cellular sources, such as, but not limited to, brain tissue biopsies, adipocytes, testes, heart, and the like. For example, cells can be obtained from an individual by biopsy and lysed, e.g., by freeze-thaw cycling, or treatment with a mild cytolytic detergent such as, but not limited to, TRITON X-1OO® polyoxyethylene ester, digitonin, IGEPAL/NONIDET P(NP)-40® (octylphenoxy)-polyethoxyethanol, saponin, and the like, or combinations thereof (see, e.g., International Patent Publication WO 92/08981, published May 29, 1992). In yet another embodiment, samples containing both cells and body fluids can be used (see ibid.).

The presence of VR-OAC in cells or in a biological fluid can be ascertained by the usual immunological procedures applicable to such determinations. A number of useful procedures are known. Three such procedures which are especially useful utilize either the VR-OAC labeled with a detectable label, antibody Ab₁ labeled with a detectable label, or antibody Ab₂ labeled with a detectable label.

The procedures and their application are all familiar to those skilled in the art and accordingly may be utilized within the scope of the present invention. For example, a “competitive” procedure, is described in U.S. Pat. Nos. 3,654,090 and 3,850,752. A “sandwich” procedure, is described in U.S. Pat. Nos. RE 31,006 and U.S. Pat. No. 4,016,043. Still other procedures are known such as the “double antibody”, or “DASP” procedure.

The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, and auramine. A particular detecting material is anti-rabbit antibody prepared in goats and conjugated with fluorescein through an isothiocyanate.

The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ⁹⁹Tc, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

In a further embodiment of this invention, test kits suitable for use by a medical specialist may be prepared to determine the presence or absence of VR-OAC in suspected target cells or biological fluids. In accordance with the testing techniques discussed above, one class of such kits will contain at least the labeled VR-OAC polypeptide or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

### Nucleic Acid-Based Diagnostics

As demonstrated in the examples, infra, nucleic acids of the invention can be used to detect defects associated with defects in the VR-OAC polypeptide associated with a phenotype comprising defects in osmotic and/or mechanical sensing function. For example, nucleic acid probes (e.g., in Northern analysis or RT-PCR analysis) can be used to determine whether a phenotype is associated with lack of expression of VR-OAC mRNA, or expression of non-functional VR-OAC mRNA, or where a mutation yields a non-transcribed mRNA. Moreover, the nucleic acid-based diagnostic techniques of the invention can be used in conjunction with antibodybased techniques to further develop a molecular understanding of phenotypes reflective of abnormal levels and activity of VR-OAC.

Human cDNA clones may be sequenced. This facilitates the determination of the complete sequence of the human gene. DNA sequences from the introns of the human VR-OAC gene may thus be obtained, and these can be used to prepare PCR primers to PCR amplify the coding sequence of the VR-OAC gene from human genomic DNA so as to identify mutations or allelic variants of the VR-OAC gene, all in accordance with protocols described in detail earlier herein.

Alternatively, the presence of microsatellites that segregate with mutant forms of human VR-OAC can be used for diagnosis. Various PCR primers, can be used in this respect.

The VR-OAC gene may also be useful diagnostically for measurements of its encoded RNA and protein in nutritional disorders. It will be of importance to know, in a particular sensory or neurological disorder, whether VR-OAC RNA and/or its encoded protein is upregulated or downregulated.

### Therapeutics

The therapeutic possibilities that are raised by the existence of VR-OAC, its activity and particular expression derive from the fact that the VR-OAC of the present invention is involved in or required for sensing and/or response to osmotic pressure, osmotic changes and mechanical stimulation. Its expression in recognized responsive centers and sensory organs implicates VR-OAC in osmoregulation and mechanoreception in key areas of the mammalian sensory system.

Systemic osmotic pressure is one of the most aggressively defended set point values in vertebrate animals. Osmoregulation by the central nervous system thus constitutes a homeostatic circuit of vital significance. Mechanoreception occurs at several pivotal locations in the vertebrate nervous system, yet the molecular identity of the receptor molecules has been unknown to date. Evidence is presented herein that VR-OAC is a vertebrate mechanoreceptive ion channel. VR-OAC acts in vitro as a poorly selective cation channel that is gated by osmotic stress and mechanical force. Among the cell types that express VR-OAC are key neurosensory cells that have been previously demonstrated to respond to mechanical stimuli. These cells include those involved in inner-ear function, sensing of systemic osmotic pressure, and somatosensory perception.

As suggested earlier and elaborated further on herein, the present invention contemplates therapeutic intervention in the cascade of reactions and responses, specifically osmoregulation and mechanoreception, in which the polypeptide receptor VR-OAC is implicated, to thereby modulate response to

Therapeutic diseases or disorders for possible treatment and therapeutic intervention via VR-OAC are contemplated based on the herein provided and disclosed osmotic sensing and mechanoreceptive activity of VR-OAC and its expression in various organs and cells involved in osmotic and mechanical recognition. Thus, VR-OAC and its agonists or antagonists have therapeutic utility in various disorders or conditions, including but not limited to the following: hearing disorders, vertigo of labyrinthine origin including motion sickness, Meniere disease, arterial hypertension, CNS disorders of fluid dysregulation (including for instance, diabetes insipidus, adipsia), neurological disorders (including ataxia due to alterations of afferent input to the CNS, and paraesthesia), male infertility, immune dysfunction with alterations of antigen presentation (including HIV infection), obesity and diabetes mellitus, chronic obstructive lung disorder, bronchial asthma, sexual dysfunction due to sensory deficits, blindness due to corneal or retinal causes, and skin disorders (including psoriasis, pemphigus vulgaris and other forms of pemphigoids, pruritus, allergic skin diseases).

The polypeptides, nucleic acids, and antibodies of the invention have significant therapeutic potential. Preferably, a therapeutically effective amount of such an agent (e.g., the polypeptide or an active fragment thereof, or DNA for gene therapy, or an antisense nucleic acid for antagonizing VR-OAC activity) is administered in a pharmaceutically acceptable carrier, diluent, or excipient.

The phrase “pharmaceutically acceptable” refers to molecular entities and compositions that are physiologically tolerable and do not typically produce an allergic or similarly untoward reaction, such as gastric upset, dizziness and the like, when administered to a human. In one embodiment, as used herein, the term “pharmaceutically acceptable” may mean approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or solution saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, Pa. (1990).

The phrase “therapeutically effective amount” is used herein to mean an amount sufficient to reduce by at least about 15%, preferably by at least 50%, more preferably by at least 90%, and most preferably prevent, a clinically significant deficit in the activity, function and response of the host. Alternatively, a therapeutically effective amount is sufficient to cause an improvement in a clinically significant condition in the host. Modulation of VR-OAC activity can be useful for increasing sensory activity (by increasing its activity) or possibly decreasing undesirably high levels of sensation (by decreasing its activity).

Reduction of VR-OAC activity (by developing antagonists, inhibitors, use of neutralizing antibodies, or antisense molecules) should result in a desired reduction in unwanted sensory stimulation.

### Polypeptide-Based Therapeutic Treatment

In the simplest analysis, the VR-OAC gene is intimately associated with osmotic and mechanical stimulation and regulation. The VR-OAC gene product, and, correspondingly, cognate molecules, may be part of a signaling pathway by which sensory and other tissue in which measurable levels of the protein are found, communicates with the brain and with other organs.

The VR-OAC polypeptide, or functionally active fragment thereof, or an analog, antagonist or agonist thereof, may be administered orally or parenterally, preferably parenterally.

The VR-OAC polypeptide, or functionally active fragment thereof, or an analog, antagonist or agonist thereof, can be delivered by intravenous, intraarterial, intraperitoneal, intramuscular, or subcutaneous routes of administration. For example, the polypeptide may be administered using intravenous infusion, an implantable osmotic pump, a transdermal patch, liposomes, or other modes of administration. Alternatively, the VR-OAC polypeptide, or functionally active fragment thereof, or an analog, antagonist or agonist thereof, properly formulated, can be administered by nasal or oral administration. A constant supply of VROAC, or functionally active fragment thereof, or an analog, antagonist or agonist thereof, can be ensured by providing a therapeutically effective dose (i.e., a dose effective to induce metabolic changes in a subject) at the necessary intervals, e.g., daily, every 12 hours, etc. These parameters will depend on the severity of the disease condition being treated, other actions, such as diet modification, that are implemented, the weight, age, and sex of the subject, and other criteria, which can be readily determined according to standard good medical practice by those of skill in the art.

In a further aspect, recombinant cells that have been transformed with the VR-OAC cDNA and that express high levels of the polypeptide can be transplanted in a subject in need of enhancement of VR-OAC activity. Preferably autologous cells transformed with VR-OAC are transplanted to avoid rejection.

In yet another aspect of the present invention, pharmaceutical compositions of the VR-OAC polypeptide, or functionally active fragment thereof, or an analog, antagonist or agonist thereof, are provided. Such pharmaceutical compositions may be for administration by injection, or for oral, pulmonary, nasal or other forms of administration. In general, comprehended by the invention are pharmaceutical compositions comprising effective amounts of protein or derivative products of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimerosal, benzyl alcohol) and bulking substances (e.g., lactose, mannitol); incorporation of the material into particulate preparations of polymeric compounds such as polylactic acid, polyglycolic acid, etc., or into liposomes. Hyaluronic acid or other anionic polymers may also be used. Such compositions may influence the physical state, stability, rate of in vivo release, and rate of in vivo clearance of the present proteins and derivatives [see, e.g., Martin, Remington's Pharmaceutical Sciences, 18th Ed. (1990, Mack Publishing Co., Easton, Pa. 18042) pages 1435-1712 which is herein incorporated by reference]. The compositions may be prepared in liquid form, or may be in dried powder, such as lyophilized form.

Contemplated for use herein are oral solid dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres [U.S. Pat. No. 4,925,673]). Liposomal encapsulation may be used and the liposomes may be derivatized with various polymers [e.g., U.S. Pat. No. 5,013,556]. A description of possible solid dosage forms for the therapeutic is given by Marshall, in Modern Pharmaceutics, Chapter 10, Banker and Rhodes ed., (1979), herein incorporated by reference. In general, the formulation will include the protein (or chemically modified protein), and inert ingredients which allow for protection against the stomach environment, and release of the biologically active material in the intestine.

Also specifically contemplated are oral dosage forms of the above derivatized proteins or compounds. Protein or compound may be chemically modified so that oral delivery of the derivative is efficacious. Generally, the chemical modification contemplated is the attachment of at least one moiety to the protein (or peptide) or molecule itself, where said moiety permits (a) inhibition of proteolysis; and (b) uptake into the blood stream from the stomach or intestine. Also desired is the increase in overall stability of the protein and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline [Abuchowski et al., 1981, supra; Newmark et al., J. Appl. Biochem., 4:185-189 (1982)]. Other polymers that could be used are poly-1,3-dioxolane and poly-1,3,6-trioxocane. Preferred for pharmaceutical usage, as indicated above, are polyethylene glycol moieties.

For the protein (or derivative, fragment, analog, agonist or antagonist) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

The therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Controlled release formulation may be desirable. The drug could be incorporated into an inert matrix which permits release by either diffusion or leaching mechanisms, e.g., chewing gums. Slowly degenerating matrices may also be incorporated into the formulation. Another form of a controlled release of this therapeutic is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Also contemplated herein is pulmonary delivery of the protein or compound. The protein (or derivative) is delivered to the lungs of a mammal while inhaling and traverses across the lung epithelial lining to the blood-stream. Other reports of this include Adjei et al., Pharmaceutical Research, 7(6):565-569 (1990); Adjei et al., International Journal of Pharmaceutics, 63:135-144 (1990) (leuprolide acetate); Braquet et al., Journal of Cardiovascular Pharmacology, 13(suppl. 5):143-146 (1989) (endothelin-1); Hubbard et al., Annals of Internal Medicine, 3(3):206-212 (1989) (α1-antitrypsin); Smith et al., J. Clin. Invest., 84:1145-1146 (1989) (α1-proteinase); Oswein et al., “Aerosolization of Proteins”, Proceedings of Symposium on Respiratory Drug Delivery II, Keystone, Colo., (March 1990) (recombinant human growth hormone); Debs et al., J. Immunol., 140:3482-3488 (1988) and Platz et al., U.S. Pat. No. 5,284,656 (granulocyte colony stimulating factor). Contemplated for use in the practice of this invention are a wide range of mechanical devices designed for pulmonary delivery of therapeutic products, including but not limited to nebulizers, metered-dose inhalers, and powder inhalers, all of which are familiar to those skilled in the art.

All such devices require the use of formulations suitable for the dispensing of protein or compound. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified protein or compound may also be prepared in different formulations depending on the type of chemical modification or the type of device employed.

Nasal delivery of the protein or compound is also contemplated. Nasal delivery allows the passage of the protein to the blood stream directly after administering the therapeutic product to the nose, without the necessity for deposition of the product in the lung. Formulations for nasal delivery include those with dextran or cyclodextran.

For all of the above molecules, as further studies are conducted, information will emerge regarding appropriate dosage levels for treatment of various conditions in various patients, and the ordinary skilled worker, considering the therapeutic context, age and general health of the recipient, will be able to ascertain the proper dosage. Generally, for injection or infusion, dosage will be between 0.01 μg of biologically active protein/kg body weight, (calculating the mass of the protein alone, without chemical modification), and 10 mg/kg (based on the same). The dosing schedule may vary, depending on the circulation half-life of the protein or derivative used, whether the polypeptide is delivered by bolus dose or continuous infusion, and the formulation used.

In yet another aspect of the present invention, methods of treatment and manufacture of a medicament are provided. Conditions alleviated by or modulated by the administration of the present derivatives are those indicated above.

### Nucleic Acid-Based Therapeutic Treatment

A functional VR-OAC gene could be introduced into human sensory cells to develop gene therapy for corresponding dysfunctions. Conversely, introduction of antisense constructs into VR-OAC expressing cells, particularly hypothalamus but also including choroid plexus, or other cells where VR-OAC is expressed, would reduce the levels of active VR-OAC polypeptide.

In one embodiment, a gene encoding an VR-OAC polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, brain tissue can be specifically targeted. Examples of particular vectors include, but are not limited to, a defective herpes virus 1 (HSV1) vector [Kaplitt et al., Molec. Cell. Neurosci., 2:320-330 (1991)], an attenuated adenovirus vector, such as the vector described by Stratford-Perricaudet et al., J. Clin. Invest., 90:626-630 (1992), and a defective adeno-associated virus vector [Samulski et al., J. Virol., 61:3096-3101 (1987); Samulski et al., J. Virol., 63:3822-3828 (1989)].

In another embodiment, the gene can be introduced in a retroviral vector [e.g., Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol., 62:1120 (1988); Temin et al., U.S. Pat. No. 5,124,263; International Patent Publication No. WO 95/07358, published Mar. 16, 1995, by Dougherty et al.; and Kuo et al., Blood, 82:845 (1993)].

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker [Feigner et al., Proc. Natl. Acad. Sci. USA, 84:7413-7417 (1987); see Mackey et al., Proc. Natl. Acad. Sci. USA, 85:8027-8031 (1988)]. The use of cationic lipids may promote encapsulation of negatively charged nucleic acids, and also promote fusion with negatively charged cell membranes [Felgner et al., Science, 337:387-388 (1989)]. The use of lipofection to introduce exogenous genes into specific organs in vivo has certain practical advantages. Molecular targeting of liposomes to specific cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting (see Mackey et al., 1988, supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter [see, e.g., Wu et al., J. Biol. Chem., 267:963-967 (1992); Wu et al., J. Biol. Chem., 263:14621-14624 (1988); Hartmut et al., Canadian Patent Application No. 2,012,311, filed Mar. 15, 1990)].

### Modulators of VR-OAC

In instances where it is desired to modulate the effects and activities of VR-OAC of the present invention, an appropriate modulator of VR-OAC could be introduced to inhibit, block, activate or enhance the activity of VR-OAC.

The identification and therapeutic use and application of agonists and antagonist to ion channels, particularly in the nervous and sensory system is well established.

The present invention contemplates screens for a modulator of VR-OAC. The present invention further contemplates screens for a modulator of VR-OAC, including wherein the activity of the ion channel is modulated. In one such embodiment, an expression vector containing the VR-OAC of the present invention, or a derivative or analog thereof, is placed into a cell in the presence of at least one agent suspected of exhibiting VR-OAC modulator activity. The cell is preferably a sensory cell. The amount of VR-OAC activity is determined and any such agent is identified as a modulator when the amount of VR-OAC activity in the presence of such agent is different than in its absence. The amount of VR-OAC activity may be determined by any number of methods, including by monitoring the flux of ions, e.g. calcium, particularly utilizing a dye or other marker which is altered or evident upon changes in ion concentration or ion flux. The vectors may be introduced by any of the methods described above.

When the amount of VR-OAC activity in the presence of the modulator is greater than in its absence, the modulator is identified as an agonist or activator of VROAC, whereas when the amount of VR-OAC activity in the presence of the modulator is less than in its absence, the modulator is identified as an antagonist or inhibitor of VR-OAC. As any person having skill in the art would recognize, such determinations as these and those below could require some form of statistical analysis, which is well within the skill in the art.

The screens and methods described above may also be utilized to identify and/or characterize general osmo-regulatory molecules or agents and/or to particularly measure mechano-stimulatory or osmotic capacity of an agent, compound or molecule. Such assays/screens may not require direct interaction with VR-OAC, but would necessitate the generation of an osmotic or mechanical stimulus sufficient for recognition by Vr-OAC and thereby activation of the VR-OAC channel.

Natural effectors found in cells expressing VR-OAC can be fractionated and tested using standard effector assays as exemplified herein, for example. Thus an agent that is identified can be a naturally occurring VR-OAC modulator. Alternatively, natural products libraries can be screened using the assays of the present invention for screening such agents.

Various screening techniques are known in the art for screening for analogs of polypeptides. Various libraries of chemicals are available. Accordingly, the present invention contemplates screening such libraries, e.g., libraries of synthetic compounds generated over years of research, libraries of natural compounds, and combinatorial libraries, as described in greater detail, infra, for analogs of VROAC. The invention contemplates screening such libraries for compounds that bind to VR-OAC. Preferably, such molecules agonize or antagonize signal transduction by VR-OAC. Thus, the present invention contemplates screens for small molecule ligands or ligand analogs and mimics, as well as screens for natural ligands that bind to and agonize or antagonize activate VR-OAC in vivo.

Knowledge of the primary sequence of the receptor, and the similarity of that sequence with proteins of known function, can provide an initial clue as to the agonists or antagonists of the protein. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Another approach uses recombinant bacteriophage to produce large libraries. Using the “phage method” [Scott et al., Science, 249:386-390 (1990); Cwirla et al., Proc. Natl. Acad. Sci. USA, 87:6378-6382 (1990); Devlin et al., Science, 249:404-406 (1990)], very large libraries can be constructed (10⁶-10⁸ chemical entities). A second approach uses primarily chemical methods, of which the Geysen method [Geysen et al., Molecular Immunology, 23:709-715 (1986); Geysen et al., J. Immunologic Method, 102:259-274 (1987)] and the recent method of Fodor et al., Science, 251:767-773 (1991) are examples. Other references [Furka et al. 14th International Congress of Biochemistry, Volume 5, Abstract FR:013 (1988); Furka, Int. J. Peptide Protein Res., 37:487-493 (1991); Houghton (U.S. Pat. No. 4,631,211, issued December 1986); and Rutter et al. (U.S. Pat. No. 5,010,175, issued Apr. 23, 1991)] describe methods to produce a mixture of peptides that can be tested as agonists or antagonists.

In another aspect, synthetic libraries [Needels et al., Proc. Natl. Acad. Sci. USA, 90:10700-10704 (1993); Lam et al., International Patent Publication No. WO 92/00252; Kocis et al., International Patent Publication No. WO 94/28028, each of which is incorporated herein by reference in its entirety], and the like can be used to screen for VR-OAC ligands according to the present invention.

The screening can be performed with recombinant cells that express VR-OAC, or alternatively, using purified receptor protein, e.g., produced recombinantly, as described above. For example, the ability of labeled, soluble, or solubilized VR10 OAC, that includes the ligand-binding portion of the molecule, to bind ligand can be used to screen libraries, as described in the foregoing references.

This invention provides antagonist or blocking agents which include but are not limited to: peptide fragments, mimetic, a nucleic acid molecule, a ribozyme, a polypeptide, a small molecule, a carbohydrate molecule, a monosaccharide, an oligosaccharide or an antibody. Also, agents which competitively block or inhibit pneumococcal bacterium are contemplated by this invention. This invention provides an agent which comprises an inorganic compound, a nucleic acid molecule, an oligonucleotide, an organic compound, a peptide, a peptidomimetic compound, or a protein which inhibits the polypeptide.

### Agricultural Applications

The VR-OAC gene can also be isolated from domestic animals, and the corresponding VR-OAC polypeptide obtained thereby. It is expected that the probe derived from the murine VR-OAC gene hybridizes to corresponding homologous coding sequences from a large number of species of animals. Any of the conditions listed above are likely to afflict lower animals as well, and the present therapeutic methods are therefore equally applicable to the treatment of animals.

### Other Applications

Due to its role and capacity in osmotic and mechanical sensing, VR-OAC is uniquely capable of use and application as a biological component or sensor in biosensor or nanotechnological devices and applications. In particular, VR-OAC can be utilized in such applications for detection and assessment of osmotic and mechanical stimuli. Thus, VR-OAC can be utilized to detect osmotic and mechanical stimuli or as the facilitating component in translating an osmotic or mechanical stimulus in nano-technological, biosensor or biorobotic devices.

Biosensors combine a biological recognition mechanism with a physical transduction technique. The transduction mechanism for high sensitivity molecular detection in nature is accomplished through specific ligand-receptor binding—induced activation of ion channels, whereby an inherent signal amplification is achieved via ion flow arising from the channel gating. Cornell et al reported the reduction of this principle to a sensing device utilizing microscopically supported synthetic bilayer membrane incorporating gramicidin ion channels (Cornell et al (1999) Novartis Found Symp 225: 231-254). Biosensors have also been generated utilizing recombinant E. coli cells expressing the enzyme organophosphorous hydrolase, for measurement of organophospate nerve agents (Mulchadani, et al (1998) Anal Chem 70(19): 4140-5; Mulchadani, et al (1998) Anal Chem 70(23): 5042-6). Frog bladder membrane, which naturally contains Na⁺ channels sensitive to blockage by paralytic shellfish toxins (tetrodotoxin (TTX), saxitoxin, gonyautoxin) was integrated in a tissue biosensor to assess Na⁺ transfer (Cheun et al (1998) Toxicon 36(10):1371-81). The tissue biosensor system was able to detect very low concentrations of TTX, at concentrations below the detection limit of the mouse bio-assay. Olfactory neurons expressing G-protein coupled receptor systems, PC12 cells expressing ligand-gated cation channels P2X2, and T2 cells expressing HLA-A2 have been applied in generating biosensors for monitoring or evaluating various physiological responses (Lundstrom et al (1998) Biosens Bioelectron 13(6): 689-695; Hazama et al (1998) 437(1): 31-5; Morgan et al (1998) Biosens Bioelectron 13(10): 1099-1105).

In addition, VR-OAC may be expressed in cells, e.g., neurons, and its capacity to recognize and respond to osmotic or mechanical stimuli can be utilized to detect any such stimulation from an external or natural source.

The invention may be better understood by reference to the following Examples, which are intended to be exemplary of the invention and not limiting thereof.

### EXAMPLE 1

### Vanilloid Receptor-Related Channel (VR-OAC) is Osmotically Activated and is a Candidate Osmotic Receptor

Cloning of VR-OAC

In an attempt to identify osmoreceptive and mechanosensitive ion channels in vertebrates, we sought vertebrate homologues of the C. elegans gene Osm-9, whose product confers sensitivity to osmotic pressure, touch, and specific odorants (Colbert et al., 1997). We also used sequence information about two vertebrate proteins with significant similarity to OSM-9, the vanilloid receptor VR1, which responds to vanilloid ligands, and the vanilloid receptor-like receptor VRL-1, which is activated by noxious temperatures (Caterina et al., 1997; Caterina and Julius, 1999; Caterina et al., 1999).

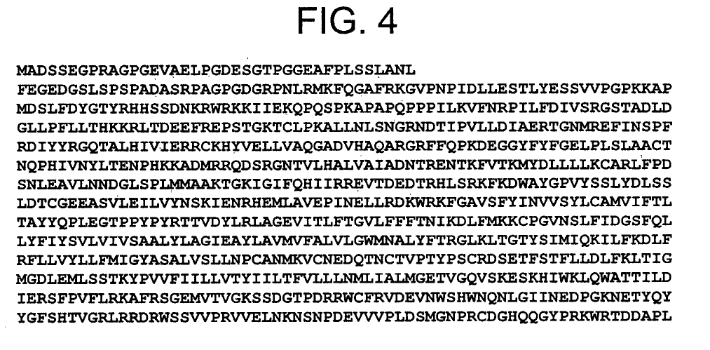

Expressed-sequence tags encoding vertebrate proteins homologous to OSM-9 and VR1 were identified in GenBank and employed as probes for high-stringency screening of a rat kidney cDNA library. This approach resulted in the isolation of a cDNA of 3,211 base pairs that includes an open reading frame of 2,613 nucleotides. In a complementary strategy, a mixture of nucleotide probes corresponding to the transmembrane regions of OSM-9, VR1, and VRL-1 was used for low-stringency screening of a mouse hypothalamic cDNA library and of an arrayed chicken innerear cDNA library (Heller et al., 1998). This led to the isolation of murine and chicken cDNAs homologous to the rat cDNA. Finally, the sequence of the human orthologue was retrieved from the high-throughput genomic-sequence database (GenBank accession number AC007834) and completed by PCR-based cloning from HEK293 cell cDNA. The nucleic acid sequence is presented in FIG. 1, and the corresponding amino acid sequence is set forth in FIG. 2. Cloning was also conducted from human kidney cells, and the nucleic acid sequence from this procedure is set forth in FIG. 3, while the corresponding amino acid sequence is presented in FIG. 4.

The novel protein identified in the four species has the primary-structural characteristics of an ion channel and is named VR-OAC, for vanilloid receptorrelated osmotically activated ion channel. The predicted amino acid sequences of VR-OAC from the four species are depicted in FIG. 5. Hydrophobicity analysis of VR-OAC indicates a structure similar to those of OSM-9, VR1, and VRL-1, with six predicted membrane-spanning domains and a putative pore loop (FIG. 6A). VR-OAC's amino-terminal domain bears three ankyrin repeats and, like its carboxyl terminus, is predicted to occur intracellularly. Analysis of phylogenetic relationships indicates that VR-OAC represents a member of the OSM-9 family in the TRP superfamily of ion channels (Harteneck et al., 2000).

The rat and mouse VR-OACs are respectively 871 and 873 amino acids in length and are 97.2% identical. Conceptual translation of the human VR-OAC sequence reveals an 871-amino-acid protein that is 94.8% identical to rat VR-OAC. The cDNA sequence of the chicken VR-OAC encodes a 852-amino acid protein that is 82.5% identical to the rat protein.

Interestingly, the VrOAC genes of both species map to chromosomal regions harboring loci for hearing disorders, bronx waltzer (by) in the mouse (Bussoli et al., 1997) and DFNA25 in the human (http://dnalab-www.uia.ac.be/dnalab/hhh). Vroac therefore constitutes a candidate gene to underlie either or both conditions.

Chromosomal Location of Vroac and Vr1 Genes

We identified genomic sequence-tagged sites flanking the human VROAC locus and mapped the gene to chromosome 12q24 between the markers D12S1339 and D12S2291. Because the human VR1 gene is located on chromosome 17 (Touchman et al., 2000), VR-OAC and VR1 stem from separate but related genes, rather than from splice variants of the same gene.

To establish the map positions of these genes in the mouse, we typed a radiationhybrid panel and a BSS interspecific cross from the Jackson Laboratory with probes corresponding respectively to Vroac and Vr1. Vroac mapped to the distal arm of chromosome 5 between the anchored markers D5Mit25 and D5Mitl88; Vr1 was localized on chromosome 11 between D11Mit7 and D11Mit36. The map positions for both genes represent chromosomal locations with conserved synteny between the murine and human genomes.

Expression of VR-OAC

To determine the expression pattern of VR-OAC, we first performed Northern blot analysis (FIG. 7). A single mRNA species of 3.2 kb was abundant in kidney, lung, spleen, testis, and fat. A lower level of expression was observed in sensory ganglia. When polyA+RNA from 150 mouse cochleae was subjected to Northern blot analysis, a faint 3.2 kb band of VR-OAC mRNA was detected (not shown).

A more detailed analysis of VR-OAC expression was performed by in situ hybridization. Although relatively faint signals are characteristically associated with low-abundance messages such as those for ion channels, we detected VR-OAC mRNA in several sites. In the central nervous system, VR-OAC was expressed in neurons of two circumventricular organs, the vascular organ of the lamina terminalis (VOLT) and the subformical organ (SFO; FIGS. 8A,B,D). The median preoptic area (MnPO) of the lamina terminalis also contained labeled neurons (FIGS. 8A,D). VR-OAC mRNA occurred in ependymal cells lining the choroid plexus of the lateral ventricles (FIGS. 4C,D); the ependymal cells of the third ventricle, on the contrary, did not express VR-OAC (FIG. 4A). Scattered neurons in other regions of the brain, including the cerebral cortex, thalamus, hippocampus, and cerebellum, expressed VR-OAC mRNA weakly (not shown). For these and all the other in situ results presented, sense controls were uniformly negative (not shown).

In the mouse's inner ear, VR-OAC was expressed in both inner and outer hair cells of the organ of Corti (FIG. 9A) as well as in hair cells in the cristae of the semicircular canals and in the utricular macula (not shown). VR-OAC mRNA also occurred in large cell bodies of the auditory ganglion and in marginal cells of the stria vascularis (FIG. 9B). Similar results were obtained for avian VR-OAC in the chicken's inner ear.

VR-OAC mRNA was abundantly expressed in the trigeminal ganglion in a subpopulation of sensory neurons with large somata (FIG. 9C). We also detected expression in Merkel cells within the sinuses of vibrissae on the snout (FIG. 9D). In the kidney, VR-OAC mRNA was found in tubular epithelial cells; a significantly weaker signal was also apparent in glomeruli (FIG. 9E).

Osmotic Gating of VR-OAC

To confirm that VR-OAC constitutes an ion channel and to determine the stimulus that gates it, we expressed the rat and chicken proteins in stably transfected Chinese hamster ovary (CHO) cells. Untransfected CHO cells and a line stably transfected with a VR1-expressing plasmid served as controls. We loaded VR-OAC-expressing cells with the Ca²⁺ indicator fluo-4 AM and used conventional and confocal icroscopy to monitor changes in fluorescence, which reflected alterations in the ntracellular Ca²⁺ concentration.

Upon osmotic stimulation of transfected cells, intracellular fluorescence increased significantly within seconds under hypotonic but not hypertonic conditions (FIG. 10A, upper panels). Cells expressing the rat and the chicken orthologues were indistinguishable in their responsiveness. A control cell line expressing rat VR1 did not respond to changes in osmotic strength but did display strong Ca²⁺ influx after exposure to the vanilloid hyperagonist resiniferatoxin (FIG. 10A, lower panels). Individual transfected cells displayed two response patterns during exposure to hypotonic conditions. Some cells had elevated intracellular Ca²⁺ levels throughout the exposure (FIG. 10B, upper trace). Other cells showed oscillating intracellular Ca²⁺ concentrations (FIG. 10B, lower trace) with as many as eight cycles of increased fluorescence during a 2-min stimulus period.

If VR-OAC is naturally gated by changes in the transmembrane osmotic pressure, one would expect the channel to respond to minute fluctuations. We confirmed this by observing responses as the osmotic strength of the extracellular medium was decreased in small increments (FIG. 10C). VR-OAC-mediated Ca²⁺ influx was detectable even upon exposure to an osmotic strength of 292 mmol kg⁻¹, a deviation of only 1% from the control value (not shown).

We performed additional experiments to test the specificity of VR-OAC's response to osmotic stimuli. Because chicken and rat VR-OAC-transfected cells did not respond to isotonic reduction of the Na⁺ concentration to as low as 50 mM, the channel is unlikely to sense hyponatremia. The cells responded neither to the vanilloid hyperagonist resiniferatoxin nor to anandamide, an endogenous cannabinoid and agonist of human and rodent VR1 (Zygmunt et al., 1999). Furthermore, no response was observed upon exposure of VR-OAC-transfected cells in isotonic medium to temperatures ranging from room temperature to 55° C.

The sensitivity of VR-OAC to osmotic stimulation was noticeably increased at physiological temperatures (FIG. 10D). Interestingly, the temperatures of greatest responsiveness differed between the rat and chicken VR-OACs. The maximal sensitivity of the rat VR-OAC occurred at the mammalian core body temperature of 37° C. For the chicken VR-OAC, maximal responsiveness corresponded to the avian core body temperature of 40° C. (Eppley, 1996). At 40° C., the difference in increase of Ca²⁺ influx between rat and chicken VR-OACs was statistically significant (p=0.03, Student's t-test). VR-OAC is therefore capable of detecting small osmotic-pressure changes under physiological conditions.

Origin of VR-OAC-Induced Ca²⁺ Signals

The large and often oscillatory Ca²⁺ signals observed after stimulation of VR-OAC-transfected cells suggest responses amplified by the release of Ca²⁺ from internal stores, which is known to occur in CHO cells (Penner et al., 1989). To confirm this possibility, we depleted internal stores in control experiments by opening IP₃-gated channels with thapsigargin; we additionally prevented Ca²⁺ replenishment by blocking potentiation channels with SKF 96365 (Merritt et al., 1990). Under these conditions, hypotonic stimuli failed to further increase the slightly elevated background level of Ca²⁺-induced fluorescence (FIG. 10E). These results suggest that VR-OACs do not admit most of the Ca²⁺ that appears in the cytoplasm upon osmotic stimulation but rather trigger Ca²⁺ release from intracellular stores.

We next sought to determine whether Ca²⁺ release from internal stores is triggered by Ca²⁺ entering the cells through open VR-OACs. When loaded with fluo-4 AM in isotonic saline solution without Ca²⁺, VR-OAC-transfected cells displayed only low baseline fluorescence (FIG. 10F). No change in fluorescence was apparent upon substitution of hypotonic solution lacking Ca²⁺; subsequent addition of 2 mM Ca²⁺ led within a few seconds to a strong increase of intracellular fluorescence. Two other experiments buttressed the conclusion that Ca²⁺ entering through VR-OACs triggers internal release. First, the fluo-4 fluorescence did not increase upon depolarization of VR-OAC-transfected cells by addition of 20 mM K⁺ to Ca²⁺-containing isotonic medium. It follows that neither depolarization per se nor any subsequent transmembrane flux of Na⁺ or K⁺ suffices to trigger release. Second, when transfected cells were maintained in a solution in which the Na+ and K⁺ had been replaced by 120 mM of the impermeant cation N-methyl-D-glucamine, but the Ca²⁺ concentration remained 2 mM, an increase in fluorescence occurred upon exposure to hypotonic solution of otherwise identical composition (not shown). These control experiments establish that Ca²⁺ entry through VR-OACs is both necessary and sufficient to evoke internal Ca²⁺ release, but do not not exclude the possibility that additional molecular signals intervene in the process.

Electrophysiological Characterization of VR-OAC

We evaluated the properties of VR-OAC by tight-seal recordings from transfected cells. Consistent with the fluorescence imaging experiments, hypotonic stress of cells during whole-cell recording induced channel opening within a few seconds to 2 min, whereas isotonic stasis or hypertonicity did not (FIG. 11A). Reversal-potential measurements indicated that the channel's permeability to K⁺ slightly exceeds that to Na⁺ and that the permeability to Cl⁻ is substantially lower.

VR-OACs displayed the dual rectification (FIG. 11B) found in some other members of the TRP superfamily (Caterina et al., 1999). Because we used EDTA to lower divalent-cation concentrations into the nanomolar range and maintained identical ionic compositions for internal and external solutions, rectification may be an intrinsic property of VR-OAC, rather than a consequence of divalent-cation blockage. Extracellular Ca²⁺ evoked a pronounced outward rectification by greatly reducing the inward current (FIGS. 11A,B). This rapid and reversible response suggests that Ca²⁺ produces a flicker block of the channel's pore. Ca²⁺ exposure also increased the outward current relative to that seen in the standard hypotonic solution. This response, which was rapid, reversible, and reproducible across many cells, betokens an additional Ca²⁺-dependent modulation of the channel.

Gadolinium ion (Gd³⁺) blocks the activity of many stretch-activated channels (Yang and Sachs, 1989). Within the first few minutes of extracellular application, the effect of 500 mM Gd³⁺ on VR-OAC-transfected cells resembled that of Ca²⁺. More protracted exposure to Gd³⁺ irreversibly abolished the whole-cell current (not shown). Similar observations were made by inside-out patch recordings. The slow development of the Gd³⁺-dependent block suggests that the ion suppresses conduction in VR-OACs through an indirect mechanism, rather than by tightly binding in the pore. Consistent results were obtained by fluo-4 imaging during Gd³⁺ application.

In patch recordings from VR-OAC-expressing cells, we observed large singlechannel currents (FIG. 7C) never encountered in control cells. On the basis of clearly defined unitary currents, the single-channel conductance of VR-OAC is 310 pS. In both inside-out and outside-out excised patches containing multiple channels, gating exhibited strong voltage dependence. Because the current during the largest voltage steps was often smaller than the response to smaller stimuli, the channels may display inactivation.

We performed a control experiment to confirm that the current through VR-OACs was unchanged under conditions in which the internal Ca²⁺ stores had been depleted. After incubation in 10 μM thapsigargin and 20 μM SKF 96365, cells displayed characteristic VR-OAC currents when stimulated with hypotonic solution. Like VR1 (Caterina et al., 1997), VR-OAC is therefore not a store-operated channel.

Deletion of the Ankyrin-Repeat Domain

The amino-terminal domain of VR-OAC includes three ankyrin repeats that might physically link the receptor to the cytoskeleton (FIGS. 5, 6A). To evaluate the potential importance of this domain, we constructed two VR-OAC variants: eGFP-OAC, which bears the complete coding sequence of rat VR-OAC fused at its amino teminus to the carboxyl terminus of enhanced green fluorescent protein (eGFP), and eGFP-OAC, a similar construct lacking the first 402 amino acids of VR-OAC, including all three ankyrin repeats. Confocal microscopy of transiently and stably transfected cells indicated that both the intact and deleted fusion proteins were localized to the plasma membrane. The fusion proteins sometimes produced membrane-associated fluorescent clusters that did not colocalize with focal adhesion points visualized by paxillin immunolabeling (not shown).

Both eGFP-OAC and eGFP-OAC Responded to Hypotonic Stimuli.

Electrophysiological evaluation of osmotically stimulated cells expressing the two fusion proteins and native VR-OAC disclosed no significant differences in their steady-state responses to hypotonic stimulation (not shown). Ca²⁺-imaging experiments revealed, however, that cells expressing the fusion protein without ankyrin repeats responded less robustly than cells expressing the intact variant during the first 60 s after application of a hypotonic stimulus. The ratio of fluorescence 60 s after stimulus application to that before stimulation differed significantly (p<0.01, Student's t-test) between cells transfected with GFP-OAC (4.4±2.1, mean±standard deviation, n=7) and eGFP-OAC (0.8±0.4, n=8).

### Discussion

Although osmoreception and mechanoreception occur at numerous locations in the vertebrate nervous system, the identity of the receptor molecules is unknown. In the above experiment, it is demonstrated that VR-OAC acts in vitro as a poorly selective vertebrate cation channel that is gated by osmotic stress. Among the cell types that express VR-OAC are neurosensory cells that have previously been demonstrated to respond to systemic osmotic pressure. Because osmosensitivity is thought to stem from the detection of membrane tension by mechanosensitive channels, VR-OACs might be expected to respond to other mechanical stimuli as well. Consistent with this hypothesis, VR-OACs occur in mechanosensory cells of the inner ear and the somatosensory system.

VR-OAC belongs to the OSM-9 family of the TRP superfamily of ion channels, whose members respond to ligands (VR1), heat (VR1 and VRL-1), and osmotic stimuli (OSM-9 and VR-OAC). In C. elegans, the single protein OSM-9 mediates responsiveness to ligands, osmotic pressure, and touch; heat has not been tested (Colbert et al., 1997). The gene ancestral to Osm-9 was presumably replicated several times during vertebrate evolution, so that the sensory functions mediated by OSM-9 have been allocated to at least three different proteins, VR1, VRL-1, and VR-OAC. VR-OAC is also distantly related to the mechanosensitive channel NOMPC in Drosophila, but has no homology with the mechanosensitive channel MIDI in yeast or with the putative channel components MEC-4 and MEC-6 in Caenorhabditis.

Gating of VR-OAC

The above experiments establish that osmotic stimuli, presumably acting through membrane stretch, efficiently gate VR-OAC. Although the response of VR-OAC to osmotic strengths slightly below the physiological setpoint supports the relevance of subtle changes in membrane tension as an effective stimulus, the gating mechanism of VR-OAC is unknown. The relatively normal sensitivity of the mutant receptor lacking the ankyrin repeats implies that an ankyrin-mediated connection to the cytoskeleton is not essential for gating by osmotic stress, but a connection might be required for rapid responsiveness. VR-OAC might be part of a multimeric complex in which a binding partner provides additional anchoring to the cytoskeleton so that VR-OAC lacking its ankyrin repeats nevertheless responds.

VR-OAC is the first osmotically activated channel to be characterized in vertebrates. A fuller understanding of its function in vivo awaits the results of analysis of gene-targeted mice. However, the functional properties obtained upon heterologous expression of VR-OAC and the localization of its mRNA suggest several possibilities that are discussed below.

Potential Relevance of VR-OAC for Systemic Osmoregulation

Systemic osmotic pressure is one of the most aggressively defended set point values in vertebrate animals. Osmoregulation by the central nervous system thus constitutes a homeostatic circuit of vital significance. In the nuclei of the central nervous system that are known to function in osmoregulation, VR-OAC is expressed in neurons of two circumventricular organs, the vascular organ of the lamina terminalis (VOLT) and the subformical organ (SFO). The circumventricular organs represent the only part of the brain lacking a blood-brain barrier (McKinley and Oldfield, 1990). VOLT and SFO neurons, which respond to changes in osmotic pressure, project to the antidiuretic hormone-secreting magnocellular neurosecretory cells in the supraoptic and paraventricular nuclei of the hypothalamus (Sibbald et al., 1988; McKinley et al., 1992; Denton et al., 1996; Bourque and Oliet, 1997). VOLT and SFO are thus regarded as osmoreceptive sensory organs within the central nervous system. Lesioning experiments have established a role for the VOLT and SFO in osmotically induced drinking behavior (Thrasher et al., 1982; McKinley et al., 1999).

In support of the hypothesis that VR-OAC serves as an osmoreceptor responsive to systemic hypotonicity, expression in the VOLT corresponds with peroxidase labeling that signals the absence of a blood-brain barrier (Bisley et al., 1996). Moreover, the sensitivity of VR-OAC is maximal at the core body temperature and lies within the physiological range of osmoregulation.

The third part of the lamina terminalis that bears VR-OAC-expressing neurons, the median preoptic area (MnPO), differs from the VOLT and SFO by possessing an intact blood-brain barrier. The MnPO has nevertheless been implicated in osmotically induced drinking behavior and in adaptive responses leading to the secretion of antidiuretic hormone (Travis and Johnson, 1993). It is plausible that MnPO neurons sense circulatory osmolality in the adjacent VOLT and SFO by means of osmoreceptor-bearing axonal projections into those areas.

The occurrence of VR-OAC in the choroid plexus of the lateral ventricles suggests a role in sensing the hydrostatic or osmotic pressure of cerebrospinal fluid (McKinley and Oldfield, 1990). The expression of VR-OAC in the kidney in tubular cells, and to a lesser degree in glomerular cells, also fits with a postulated osmosensory function of the channel.

Potential Relevance of VR-OAC for Inner-Ear Function

In the cochlea, expression of VR-OAC mRNA was detected in marginal cells of the stria vascularis, non-sensory cells that maintain the ionic composition of endolymph and the endocochlear potential. VR-OAC in these cells could participate in regulation of the osmotic or hydrostatic pressure in the endolymph. VR-OAC mRNA was also detected in cochlear outer and inner hair cells and in vestibular hair cells. The channel might therefore act as an osmotic sensor in the fluid homeostasis of these cells.

Might VR-OAC form the hair cell's mechanoelectrical transduction channel? The channel's physiological properties, including voltage sensitivity and a single-channel conductance of 310 pS, make this role improbable for VR-OAC alone. The VR-OAC protein could, however, be part of a heteromultimeric transduction channel. It might alternatively mediate a second form of mechanosensitivity in hair cells, the slow mechanical response that persists after tip-link disruption (Meyer et al., 1998). It is interesting in this context that nompC null mutants display a small mechanoreceptor potential (Walker et al., 2000) that might be caused by the Drosophila orthologue of VR-OAC. More generally, the data and discussion in Example 2, below, reflects the determination that VR-OAC does act as a mechanoreceptor.

Potential Relevance of VR-OAC for Somatosensory Function

VR-OAC mRNA is abundantly expressed by a subpopulation of large neurons in the trigeminal ganglion. The labeled cells include neither the small sensory neurons that give rise to unmyelinated C fibers nor the very large neurons whose Aα (class I) fibers innervate muscle spindles. VR-OAC mRNA occurs in neurons whose myelinated, rapidly conducting Aγ (class 11) and Aδ (class III) fibers mediate epicritic mechanosensation. It is noteworthy that the maximal sensitivity of VR-OAC at normal body temperatures (FIG. 6D) corresponds to the peak thermal sensitivity for tactile and vibratory stimuli in mammals (Weitz, 1941; Fucci et al., 1976; Dehnhardt et al., 1998).

We also observed VR-OAC mRNA in the Merkel cells associated with vibrissae. Because of their synaptic contacts with sensory nerve endings, Merkel cells have been suggested to be mechanoreceptors (Andres and von Düring, 1973).

### EXAMPLE 2

### Vanilloid Receptor-Related Channel is Mechanically Activated and is a Candidate Vertebrate Mechanoreceptor

Summary

As discussed above, mechanoreceptive proteins have heretofore never been identified in vertebrates. The experiment and findings that follow below reveal and substantiate the discovery by the present inventors of just such a mechanoreceptor in the elucidation and testing of VR-OAC. Because this channel is expressed in key neurosensory cells, including inner-ear hair cells and neurons of circumventricular organs and sensory ganglia, VR-OAC is a strong candidate to mediate mechanical responsiveness in vertebrates. The expression pattern of this channel, as well as its functional properties upon heterologous expression in eukaryotic cells, suggest that it is a vertebrate sensory mechanoreceptor involved in inner-ear function, mechanical extero- and interoception, and osmotic sensitivity.

### Results

Direct Mechanical Stimulation of VR-OAC

To investigate the mechanical sensitivity of cell lines stably expressing VR-OAC, we gently pipetted a 100 μL drop of isotonic medium onto the cellular monolayer from a distance of 5 mm. An increase in Ca2+ influx, as measured by fluo-4 fluorescence, was readily apparent within a few seconds at room temperature (not shown). In agreement with the earlier experiments on temperature sensitivity, we noted a much stronger increase in Ca2 + influx when this experiment was conducted at 37° C. No change in the fluorescence signal ensued with VR1-transfected cells or with untransfected controls. These data suggest that VR-OAC is gated by direct force application.

To document mechanosensitivity in VR-OAC-expressing cells more directly and with better time resolution, we recorded from individual cells with tight-seal electrodes in the whole-cell configuration. In one type of experiment, we used a solenoid-controlled pressure source to direct a jet of saline solution at each cell. This mechanical stimulus elicited an inwardly directed current ranging from about −20 pA to more than −1000 pA (FIG. 11A). No such currents were observed after comparable stimulation of untransfected cells, so the response did not reflect artifacts such as deterioration of the tight seal. Control experiments indicated that fluid flow at the cell lagged the electrical command signal by 11 ms. Because the minimal latencies of the electrical responses in these experiments were 11-16 ms, no more than a few milliseconds elapsed between the application of a mechanical stimulus to a cell and the opening of VR-OACs.

In a second set of experiments, individual cells were directly stimulated during whole-cell recording by drawing a flame-polished glass probe across their surfaces. At a holding potential of −60 mV, a VR-OAC-transfected cell responded to such stimuli within 1 ms with inward currents of up to −30 pA. The responses were qualitatively similar to those obtained with similar stimuli from stretch-activated channels (Bett and Sachs, 2000). Untransfected cells again failed to respond. Taken together, these results indicate that mechanical stimulation, whether by the force of a fluid jet or by direct contact, rapidly opens VR-OACs.

### Discussion

Mechanoreception occurs at several pivotal locations in the vertebrate nervous system, yet the molecular identity of the receptor molecules is unknown. In the above experiments, evidence is presented that VR-OAC is a vertebrate mechanoreceptive ion channel. VR-OAC acts in vitro as a poorly selective cation channel that is gated by osmotic stress and mechanical force. Among the cell types that express VR-OAC are key neurosensory cells that have been previously demonstrated to respond to mechanical stimuli. These cells include those involved in inner-ear function, sensing of systemic osmotic pressure, and somatosensory perception.

VR-OAC belongs to the OSM-9-like family of the TRP superfamily of ion channels, whose members respond to ligands (VR1), heat (VR1, VRL-1), and now mechanical stimuli (VR-OAC). Interestingly, the single gene Osm-9 of C. elegans mediates responsiveness to ligands, osmotic pressure, and touch; heat has not been tested (Colbert et al., 1997). The recently completed sequencing of the Drosophila genome revealed the presence of several genes related to Osm-9, one of which is probably its orthologue (FIGS. 5 and 6B). The Osm-9 ancestor was presumably replicated several times during Drosophila evolution, allowing different yet related forms to respond to distinct sensory modalities. A similar phenomenon seems likely to have occurred in vertebrates, in which the sensory functions mediated by Osm-9 have been allocated to at least three different genes, Vr1, Vrl1, and Vroac. VR-OAC is also distantly related to the mechanosensitive channel NompC in Drosophila, but has no homology with the mechanosensitive channel Midl in yeast or with the putative channel components mec-4 and mec-6 in Caenorhabditis.

Gating of VR-OAC

Our experimental findings establish that osmotic stimuli, presumably acting through membrane stretch, suffice to gate VR-OAC. The observations that the channel also responds to mechanical force and that it is insensitive to changes in the extracellular Na+ concentration accord with the possibility that the channel is directly gated by changes in membrane tension. The fact that VR-OAC responds to osmotic strengths differing only slightly from the physiological setpoint further supports the relevance of subtle changes in membrane tension as the effective stimulus.

The gating mechanism of VR-OAC is unknown. The relatively normal sensitivity of the mutant receptor lacking the ankyrin repeats implies that an ankyrin-mediated connection to the cytoskeleton is not essential for gating by osmotic stress. An ankyrin-mediated connection to the cytoskeleton may be necessary, however, for rapid responsiveness. VR-OAC might be part of a multimeric complex, in which a hypothetical binding partner provides additional anchoring to the cytoskeleton so that VR-OAC lacking its ankyrin repeats nevertheless responds.

VR-OAC is the first mechanically activated channel to be characterized in vertebrates. A fuller understanding of its function in vivo awaits the results of analysis of gene-targeted mice. However, the functional properties obtained upon heterologous expression of VR-OAC and the localization of its mRNA in the ear, anterior hypothalamus, trigeminal ganglion, and Merkel cells suggest a number of possibilities that are discussed below.

Potential Relevance of VR-OAC for Inner-Ear Function

VR-OAC mRNA is expressed in cochlear outer and inner hair cells and in vestibular hair cells. Might VR-OAC form the hair cell's mechanoelectrical transduction channel? The channel's physiological properties, including voltage sensitivity and a single-channel conductance of 310 pS, make this role improbable for VR-OAC alone. The VR-OAC protein could, however, be part of a heteromultimeric transduction channel. It might alternatively mediate a second form of mechanosensitivity in hair cells. Removal of tip links, the elastic structures thought to gate the hair cell's transduction channels, largely extinguishes the hair cell's mechanosensitivity. The slow residual mechanical response (Meyer et al., 1998) might be due to VR-OACs. It is interesting in this context that nompC null mutants display a small mechanoreceptor potential (Walker et al., 2000), which might be caused by the Drosophila orthologue of VR-OAC (FIG. 5).

In the cochlea, expression of VR-OAC mRNA was also detected in marginal cells of the stria vascularis, non-sensory cells that maintain the ionic composition of endolymph and the endocochlear potential. VR-OAC in these cells could participate in regulation of the osmotic pressure or fluid pressure in the endolymph. VR-OAC might also act as an osmotic sensor in the fluid homeostasis of hair cells.

Potential Relevance of VR-OAC for Somatosensorv Mechanoreception

Somatosensory mechanoreception is a feature of all body surfaces and accessory structures. The sensory neurons for touch perception and related modalities are localized in sensory ganglia (Gardner et al., 2000). We demonstrated by in situ hybridization that VR-OAC mRNA is abundantly expressed by a subpopulation of large neurons in the trigeminal ganglion. The labeled cells belong neither to the class of small sensory neurons that give rise to unmyelinated C fibers nor to the class of very large neurons whose Aa (class I) fibers innervate muscle spindles. VR-OAC mRNA was detected in neurons whose myelinated, rapidly conducting Ag (class II) and Ad (class III) fibers mediate epicritic mechanosensation. Which of the mechanical submodalities—touch, vibration, and positional sense—might be mediated by VR-OAC cannot yet be determined. It is noteworthy, however, that the maximal sensitivity of VR-OAC at normal body temperatures (FIG. 10D) corresponds to the peak thermal sensitivity for tactile and vibratory stimuli in mammals (Weitz, 1941; Fucci et al., 1976; Dehnhardt et al., 1998).

We also observed VR-OAC mRNA in the Merkel cells associated with vibrissae. Because of their synaptic contacts with sensory nerve endings, Merkel cells have been implicated as mechanoreceptors (Andres and von Düring, 1973; but see Gardner et al., 2000); they might therefore possess mechanically sensitive channels.

VR-OAC mRNA occurs not only in neurosensory cells and the kidney, but also in lung, testis, spleen, fat, and scattered neurons in the central nervous system outside the lamina terminalis.

### EXAMPLE 3

### In situ Hybridization Studies

Additional studies were performed in accordance with the procedures and protocols set forth in Example 1, above, in an effort to further confirm the presence of VR-OAC and consequently, the role that the receptor of the invention plays in the mediation and transmission of mechanical stimuli. Specifically, FIGS. 13 and 14 are panel photographs presenting expression data confirming the presence of VR-OAC in various compartments/organs.

The panel shows in situ hybridization of rodent tissue samples with nucleotide probes specific for VR-OAC in FIGS. 13A-J, as follows:

A. Mouse lung. VR-OAC is expressed in lung tissue, in alveolar cells. Gene expression of an osmotically activated ion channel in alveolar cells points towards a role for this receptor in the fluid- and osmotic regulation of these cells, possibly their reactivity to mechanical stimuli of lung tissue. Therefore, VR-OAC might be important in the pathogenesis of alveolar lung disease such as pulmonary edema and

allergic alveolitis. Through influence on intimately associated smallest airways, a role in bronchial asthma and chronic obstructive lung disease can be assumed.

B. Mouse spleen. VR-OAC is strongly expressed in cells resembling macrophages and follicular dendritic cells, key antigen presenting cells of the immune system. These cells are pivotal antigen presenting cells in the immune system. VR-OAC can reasonably be assumed to play a role in control of movement of these cells and their processes. Follicular dendritic cells of the spleen and splenic macrophages are known to play a key role in HIV infection, including AIDS, and in autoimmune diseases such as rheumatoid arthritis. Since locomotion of these cells including extension of their processes is a vital factor for their functioning, VR-OAC could be a key molecule for the proper functioning of these immune cells and it could play a role in the above diseases.

C. Rat testis. VR-OAC is heavily expressed in spermatocytes. A role for VROAC in spermatocyte/sperm motility is suggested and a dysfunction of VR-OAC could be a cause for male infertility.

D. Rat snout skin. VR-OAC is expressed in Merkel cells in the sinus of vibrissae and also in the vicinity of smaller hairs. VR-OAC is also expressed in touch-sensitive Merkel cells associated with epidermis and in epidermal cells. Merkel cells represent mechanotransductory cells in the skin which form synapses with innervating nerve fibres. VR-OAC could either participate in mechanoreception as part of a mechanosensory transduction channel or as an osmoregulator of osmotically challenged sensory cells such as hair cells in the ear or Merkel cells in the skin. Dysfunction of VR-OAC could be the cause for altered sensation in the skin which could be the reason for dysaesthesia, paraesthesia [and anaesthesia] and pruritus. With respect to the gene expression [albeit at lower levels] of VR-OAC in skin epidermal cells, VR-OAC, as an osmotic sensor for these mechanically and osmotically most challenged cells, could serve in maintaining the integrity of the skin/epidermis. Thus, skin diseases with dysfunction in this respect could be caused by dysfunction of VR-OAC. Such disesaes include psoriasis; pemphigus vulgaris and other forms of pemphigoids; allergic skin diseases, severe skin diseases associated with burns and other wounds/trauma.

E. Rat white adipose tissue (WAT). VR-OAC is expressed in adipocytes. VR-OAC could possibly help maintain the osmotic equilibrium of adipocytes from WAT. That alone renders VR-OAC as a candidate molecule in the pathogenesis of obesity. But another, more attractive scenario of obesity pathogenesis appears likely. VR-OAC can sense the membrane stretch of an adipocyte which is determined by how much lipid the adipocyte has stored intracellularly. The more lipid inside the cell, the higher the tension in the membrane. This would lead to gating of VR-OAC with subsequent calcium ion influx. In this respect, the adipocyte can not only be regarded as a storage cells for fat, but rather can be viewed as an endocrine cell which produces a certain amount of the key weightregulating hormone, leptin, which signals in an “adipostat” homeostatic circuit to the hypothalamus, leading to reduced food intake. It is known from related endocrine cells, namely pancreas islet beta cells, which produce insulin, that calcium influx is a key event in the regulation of insulin secretion. Likewise, VR-OAC could lead to calcium influx into adipocytes regulating, as a key signaling event, the secretion of leptin. Leptin is known to regulate food intake and glucose and lipid metabolism. Therefore, the pathogenesis of obesity and diabetes mellitus are linked to the functioning of VR-OAC in adipocytes.

F. Mouse orbital tissue. VR-OAC is expressed in orbital adipocytes. Similar observations were made in orbital adipose tissue. This adipose tissue does not so much play a role in obesity, but rather in endocrine orbitopathy associated with thyroid disease.

G. Mouse cornea. VR-OAC is expressed in corneal squamous epithelial cells. It was also found in the angle of the anterior chamber of the eye (not shown). VROAC could sense changes in osmotic pressure on the surface of the cornea. Therefore, VR-OAC could be key for the integrity and maintenance of integrity of the cornea. It appears likely that the pathogenesis of corneal diseases with compromised structural integrity/maintenance thereof could be linked to the functioning of VR-OAC. Such diseases or conditions include corneal ulceration, herpes virus infections of the cornea, congenital corneal dystrophies, corneal trauma [mechanical, burn, chemical], other corneal infectious diseases, and corneal transplantation. Alternatively, VR-OAC could participate in mechanoreception of the cornea. Altered mechanoreception of the cornea could conceivably be linked to dysfunction of VR-OAC. Such altered mechanoreception, e.g. hypersensitivity, is a common problem in human subjects wearing contact lenses, but also in the ophthalmological manifestation of allergic rhinitis/conjunctivitis [hayfever]. With respect to the weak, but nevertheless significant expression of VR-OAC in the angle of the anterior chamber of the eye, an osmoregulatory role of VR-OAC in the production of anterior chamber fluid is suggested. The disease associated with dysfunction of this delicate process is glaucoma.

H. Mouse retina. VR-OAC is expressed in photoreceptors and retinal ganglion cells. VR-OAC could, as in inner ear hair cells and Merkel cells of the skin, serve as an osmoregulatory ion channel for the photoreceptors in which it is expressed. Malfunction of VR-OAC could obviously be the cause for malfunction and death of these non-replacable cells and subsequently cause impaired vision or even blindness.

I. Mouse brain. VR-OAC is expressed in nerve-cells of the hippocampus, CA1 region, a region of importance for memory and in epileptic seizures. VR-OAC could serve as an osmoregulatory ion channel for the hippocampal neurons which have been shown to be particularly involved in the formation of memory, but also in the pathogenesis of epilepsy. Thus, diseases with memory impairment such as dementias, but also seizure disorders could be linked to malfunctioning of VR-OAC.

J. Mouse brain. VR-OAC is expressed in cerebellar nerve cells. All sections with mouse tissue have been recapitulated with rat tissue and vice versa. With a protein sequence similarity of 94.8% between rat and human, a similar gene expression profile can be reasonably assumed to be detected in human tissue. VROAC could serve as an osmoregulatory ion channel for cerebellar neurons. These neurons have been shown to play a role in coordination including limb-muscle coordination/stability of gait and precision of arm and finger movements, eyemovement coordination, coordination of the muscular apparatus producing speech and even coordination of emotions. Thus, malfunction of VR-OAC in cerebellar neurons could possibly lead to dyscoordination of the full spectrum of coordination implemented through the cerebellum.

Additional expression data in FIGS. 14A-F confirms the presence of VR-OAC in the following compartments/organs:

A, A′) Albino rat snout vibrissa (V)

A) An in situ hybridization with a VR-OAC antisense cRNA. The blue specific signal can be detected in Merkel cells (MeC). The black deposit stems from immunolabeling for neurofilament protein.

A′) No specific signal is obtained with a sense control cRNA.

No counterstain.

B, B′) Mouse central nervous system, subformical organ

B) In an in situ hybridization with a VR-OAC antisense cRNA, specific labeling can be detected in neurons of the subformical organ (SFO). For orientation: corpus callosum (CC).

B′) No specific signal occurs when a sense control cRNA is used.

Light counterstain with nuclear fast red.

C, C′) Rat central nervous system, lateral ventricle (LV) with choroid plexus (CP)

C) In an in situ hybridization with a VR-OAC antisense CRNA, specific labeling is detected in ependymal cells of the choroid plexus. No counterstain.

C′) No specific signal occurs when a sense control cRNA is used. Light counterstain with nuclear fast red.

D, D′) Mouse renal cortex

D) In an in situ hybridization with a VR-OAC antisense cRNA, a specific signal is detectable in tubular epithelial cells (T) and to a much lesser extent in glomeruli (G).

D′) No specific signal is detected when a sense control cRNA was employed. Light counterstain with nuclear fast red.

E) Longitudinal section, mouse inner ear, organ of Corti

An in situ hybridization with a mouse VR-OAC antisense cRNA shows intense apical labeling in tangentially sectioned outer hair cells. No counterstain.

F) Chicken inner ear, cochlea

In an in situ hybridization of the chicken's cochlea with chicken VR-OAC antisense cRNA, a specific signal occurs in hair cells (HC) and in cells of the tegmentum vasculosum (TV), the chicken equivalent of the mammalian stria vascularis. No counterstain.

The above hybridization studies and their results further establish and support the roles set forth for the receptor of the present invention as both an osmoreceptor and a mechanoreceptor. These findings portend significant advances in the understanding of sensory signal reception and transmission, among other applicable areas, and offer the hope of remediation of sensory deficits resulting from trauma, disease or genetic deficiency. Therapies would extend to and include transplantation and gene therapy to restore or appropriately modulate channel function and activity, and all of the areas and compartments discussed and listed herein are contemplated within the spirit and scope of the invention.

### Experimental Procedures

The following discusses common procedures that are used in connection with the above examples and experiments presented.

Cloning of VR-OAC

10⁵ clones of a rat kidney cDNA library (Tate et al., 1992) were screened with a 283-base-pair fragment derived from W53556, an EST with homology to VR1. This led to the direct isolation of rat VR-OAC. 2 10⁶ clones of a mouse hypothalamus cDNA library, constructed in the 1-phage vector 1-ZAP (Stratagene Inc., La Jolla, Calif.), were screened with a mixture of nucleotide probes corresponding to the transmembrane regions of VR1, VRL-1, and OSM-9. Two 2.6-kb clones harbored an incomplete mouse VR-OAC cDNA. The missing 5′ end was retrieved through 5′-RACE (Clontech Laboratories, Palo Alto, Calif.).

221,184 randomly selected clones from a chicken auditory epithelium cDNA library (Heller et al., 1998) were arrayed on nylon filters (Genome Systems/Incyte Genomics Inc., St. Louis, Mo.). The resulting macroarrays were screened with 33P-labeled probes in a fashion similar to that used for the hypothalamic cDNA library. Analysis of the arrays was performed using a phosphoimager (STORM 840, Molecular Dynamics, Sunnyvale, Calif.) and Array Vision software (Image Research Inc., St. Catharines, Ontario, Canada). One clone contained the full-length coding sequence of chicken VR-OAC. Six additional chicken VR-OAC cDNAs were isolated by conventional plaque hybridization from the original chicken library.

cDNAs were sequenced in the Protein/DNA Technology Center of The Rockefeller University. Amino-acid sequences were analyzed using the PSI-BLAST algorithm (http://www.ncbi.nlm.gov), PSORT (http://psort.nibb.ac.jp), the PHDsec program (Rost and Sander, 1993), Kyte-Doolittle hydropathy analysis (Kyte and Doolittle, 1982), and PHYLIP (Retief, 2000).

Expression Analysis

Northern blot analysis was performed by established protocols (http://www.ambion.com/techlib/index.html) using 32P-dCTP labeled cDNA probes corresponding to nucleotides 384-667 of rat VrOAC, and, for higher sensitivity, digoxigenin-UTP labeled cRNA probes. The latter probes were detected with a chemiluminescence kit (Roche, Basel, Switzerland). polyA+RNA was isolated from total RNA using the Oligotex kit (Qiagen, Hilden, Germany).

In situ hybridization was performed according to established protocols (Heller et al., 1998; http://dir.niehs.nih.gov/dirlep/ish.html). Frozen sections were cut at thicknesses of 6-14 μm with a cryomicrotome (CM3000, Leica GmbH, Nussloch, Germany), mounted on slides, and fixed in 4% paraformaldehyde. Digoxigenin-UTP-labeled riboprobes were immunodetected after 120-min washes at 65°-70° C. with 50% (v/v) formamide in 150 mM NaCl and 15 mM trisodium citrate at pH 7 (1× SSC) and in 0.1× SSC. Two mouse VR-MAC-specific antisense ribroprobes were synthesized, one corresponding to nucleotides 384-667, encoding the amino terminus, and another corresponding to nucleotides 1401-1746, encoding the first two transmembrane domains. The respective sense probes were used as controls. Additional negative control experiments employed RNAse-digested sections or omitted the anti-digoxigenin antibody. Ear and sensory ganglion specimens were examined in both chickens and mice. For the remaining organs, only mice and rats were examined. Immunocytochemistry was performed according to established protocols (Liedtke et al., 1996) and the manufacturers' suggestions (zymed.com; http://home.att.net/^{˜}sternbmonoc/SMI35.htm).

Chromosomal Mapping

Radiation hybrid panel mapping was conducted with the T31 mouse-hamster genomic DNA hybrid cell line panel (Research Genetics, Huntsville, Ala.). A mouse-hamster polymorphism based on VrOAC nucleotides 384-501 was used for the PCR. Results were submitted to the Jackson Laboratory mouse radiation hybrid (http://www.jax.org/resources/documents/cmdata/rhmap/RHIntro.html). Despite several attempts, this approach proved futile for mapping Vr1. To map Vrl, we amplified from mouse genomic DNA a 1.6-kb intronic sequence between nucleotides 2082 and 2346. Using as templates Mus spretus and M. musculus BL6 genomic DNA, we employed an XbaI polymorphism to type the JAX BSS backcross panel, (C57BL/6JEi×SPRET/Ei)F1×SPRET/Ei (Rowe et al., 1994). Results were submitted to the Jackson Laboratory database server (http://www.jax.org/resources/documents/cmdata/).

Cell Lines and Ca2+-Imaging Experiments

The complete cDNAs of rat VR-OAC, chicken VR-OAC, and rat VR1 were subcloned into the eukaryotic expression vector pcDNA3.1 (Invitrogen, Carlsbad, Calif.). CHO-K1 cells (ATCC, Manassas, Va.) were transfected with these vectors and selected with G418 (Life Technology, Gaithersburg, Md.). Stable clones that expressed the mRNAs were identified by Northern blot analysis and, for VR1, by response to ligand.

For Ca2+ imaging, cells were loaded for 30 min at room temperature in 1.5 mM fluo-4 AM ester (Molecular Probes, Eugene, Oreg.) in isoosmotic Hille's saline solution (130 mM Na⁺, 2.5 mM K⁺, 2 mM Ca2+, 1 mM Mg2+, 138.5 mM Cl−, 20 mM D-glucose, and 10 mM HEPES at pH 7.3; Babcock et al., 1997) containing 2 mM probenecid. The cells were then washed and incubated for 15 min at 30° C. to promote deacetylation of internalized fluo-4 AM ester.

Confocal analysis was performed with an MRC-1024ES system (Bio-Rad, Hercules, Calif.) mounted on an inverted microscope (Zeiss Axiovert 135TV, Carl Zeiss, Jena, Germany). Confocal image series were collected using Lasersharp software (BioRad) running on a Poweredge 2300 computer (Dell Computer, Round Rock, Tex.). Images were also recorded with a cooled digital camera (Micromax, Princeton Instruments Inc., Princeton, N.J.) atop a Zeiss Axioplan microscope. These images were analyzed using IPLab software (Scanalytics Inc., Fairfax, Va.). Analysis of single-frame or single-cell integrated signal density was performed on Macintosh computers running NIH Image software (version 1.61; O'Neill et al., 1989).

Solutions with different osmotic strengths were created by adding water or mannitol to Hille's saline solution. Unless otherwise noted, the Ca2+ concentration of all solutions was held constant at 2 mM. The resultant osmolalities were verified with a vapor-pressure osmometer (Vapro 5520, Wescor Inc., Logan, Utah). The test solutions included a graded series of hypotonic solutions (with 220 mmol kg-1 the lowest osmolality), a hypertonic solution (330 mmol kg-1), isotonic solutions at 295 mmol kg-1 with graded concentrations of Na+ (with 50 mM the lowest concentration), and hypotonic solutions containing 100 μM, 250 μM, and 500 μM GdCl3. Ligands used for stimulation were resiniferatoxin (Sigma Chemicals, St. Louis, Mo.) at 0.2 μM and 5 μM and anandamide (Cayman Chemical, Ann Arbor, Mich.) at 5 μM and 25 μM. To test whether VR-OAC is directly gated by increases in temperature, we exposed dishes containing fluo-4-loaded cells to temperatures ranging from room temperature to 30° C., 37° C., 40° C., 45° C., 50° C., and 55° C. Images were acquired during the thermal stimulus series at intervals of 60 s for up to 10 min after the start of heating. A series of calibration experiments confirmed that this interval ensured proper thermal equilibration. A similar procedure was used to test the response to hypotonicity at various temperatures.

Electrophysiological Recordings

Electrophysiological characterization of VR-OAC was performed at room temperature on CHO-K1 cell lines stably or transiently transfected with one of four expression vectors, pcDNA3.1 (Invitrogen) with inserts bearing chicken or rat VR-OAC, eGFP-OAC, or eGFP-ΔOAC. These cells were selected because untransfected HEK 293 cells responded to hypotonic stress and Xenopus laevis oocytes are known to respond to membrane stretch (Yang and Sachs, 1989). Cells plated on glass-bottom Petri dishes were observed through a 40× water-immersion objective lens on an inverted microscope (Aviovert 100 M, Carl Zeiss) equipped with Nomarski optics. We recorded whole-cell and patch currents with a voltage-clamp amplifier (EPC-7, List-Electronic, Darmstadt-Eberstadt, Germany). Voltage stimuli were generated and responses recorded with a computer (P6400 GX1, Dell) running LabVIEW 5.0 software (National Instruments, Austin, Tex.). Voltage and current responses were low-pass filtered at 2 kHz with an eight-pole Bessel filter and sampled at 5 kHz.

Tight-seal pipettes were bent to permit an orthogonal approach to a cell's surface and heat-polished to give resistances of 3-5 MΩ. Internal solutions typically contained 117 mM NaCl, 5 mM HEPES, 1 mM EDTA, and mannitol to achieve isotonicity. EDTA was omitted in the pipette solution during experiments in which the bath solution also lacked EDTA. Pipette and cell capacitance were largely compensated; series resistance compensation ranged from 10% to 60%. Ground reference was provided by an Ag—AgCl electrode through a 150 mM KC1 agar bridge.

A fluid-jet stimulus was delivered by applying a pressure pulse of about 3 kPa to the rear of a glass pipette with a tip diameter of 5-10 μm. Filled with saline solution identical to that in the recording chamber, the pipette's tip was oriented perpendicular to the bottom of the chamber and placed roughly 10 μm above the target cell. The onset and termination of the jet were regulated by a solenoid valve (Picospritzer II, General Valve Corporation, Fairfield, N.J.) under computer control. During whole-cell recording, cells were maintained in isotonic or slightly hypertonic solutions. Mechanical stimulation was accomplished by perfusion of hypotonic solutions. The standard hypotonic solution contained 117 mM NaCl, 5 mM HEPES, and 1 mM EDTA and had an osmotic strength of 225 mmol kg-1. Isotonic and hypertonic solutions were prepared by supplementing the standard hypotonic solution with mannitol to respectively 295 mmol kg−1 and 340 mmol kg−1. All solutions used in perfusion of cells had a pH of 7.3 and contained 1 mM phenol red for visibility. EDTA did not appear to have deleterious effects on the cells, and whole-cell currents could be recorded stably for periods over 20 minutes.

In studies of the effects of Ca2+ and Gd3+ on whole-cell currents, hypotonic test solutions included respectively 2 mM CaCl2 or 500 mM GdCl3. Because carboxylic-acid Ca2+ chelators are known to compromise the capacity of GdC13 to block mechanosensitive channels (Caldwell et al., 1998), EDTA was excluded from the latter medium and the pipette solution. Inside-out and outside-out patch recordings were performed with isotonic NaCl solution without EDTA in the pipette and an identical solution supplemented with 1 mM CaC12 in the bath.

GenBank Accession Numbers

The rat, mouse, human, and chicken cDNA sequences for VR-OAC described in this paper are entered in GenBank under accession numbers AF263521, AF263522, AF263523, and AF261883, respectively.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Where nucleotide or amino acid sequence lengths are provided, or molecular weight values given, they are approximate.

Various publications are cited herein, the disclosures of which are incorporated by reference in their entireties. The following references are identified in short form in the foregoing specification, and are listed in full below.

Andres, K. H., and von Düring, M. (1973). Morphology of cutaneous receptors. In Handbook of Sensory Physiology, Volume II, Somatosensory System, A. Iggo, ed. (Berlin, Germany: Springer), pp. 3-28.

Babcock, D. F., Herrington, J., Goodwin, P. C., Park, Y. B., and Hille, B. (1997). Mitochondrial participation in the intracellular Ca2+ network. J Cell Biol 136, 833-844.

Bett, G. C. L., and Sachs, F. (2000). Whole-cell mechanosensitive currents in rat ventricular myocytes activated by direct stimulation. J Membr Biol 173, 255-263.

Bisley, J. W., Rees, S. M., McKinley, M. J., Hards, D. K., and Oldfield, B. J. (1996). Identification of osmoresponsive neurons in the forebrain of the rat: a Fos study at the ultrastructural level. Brain Res 720, 25-34.

Bourque, C. W., and Oliet, S. H. (1997). Osmoreceptors in the central nervous system. Annu Rev Physiol 59, 601-619.

Caldwell, R. A., Clemo, H. F., and Baumgarten, C. M. (1998). Using gadolinium to identify stretch-activated channels: technical considerations. Am J Physiol 275, 619-C621.

Caterina, M. J., and Julius, D. (1999). Sense and specificity: a molecular identity for nociceptors. Curr Opin Neurobiol 9, 525-530.

Caterina, M. J., Rosen, T. A., Tominaga, M., Brake, A. J., and Julius, D. (1999). A capsaicin-receptor homologue with a high threshold for noxious heat. Nature 398, 436-441.

Caterina, M. J., Schumacher, M. A., Tominaga, M., Rosen, T. A., Levine, J. D., and Julius, D. (1997). The capsaicin receptor: a heat-activated ion channel in the pain pathway. Nature 389, 816-24.

Colbert, H. A., Smith, T. L., and Bargmann, C. I. (1997). OSM-9, a novel protein with structural similarity to channels, is required for olfaction, mechanosensation, and olfactory adaptation in Caenorhabditis elegans. J Neurosci 17, 8259-8269.

Dehnhardt, G., Mauck, B., and Hyvarinen, H. (1998). Ambient temperature does not affect the tactile sensitivity of mystacial vibrissae in harbour seals. J Exp Biol 201, 3023-3029.

Denton, D. A., McKinley, M. J., and Weisinger, R. S. (1996). Hypothalamic integration of body fluid regulation. Proc Natl Acad Sci USA 93, 7397-7404.

Eppley, Z. (1996). Development of thermoregulation in birds: physiology, interspecific variation and adaptation to climate. In Animals and Temperature. Phenotypic and Evolutionary Adaptation. I. A. Johnston and A. F. Bennett, eds. (Cambridge, United Kingdom: Cambridge University Press), pp. 313-345.

French, A. S. (1992). Mechanotransduction. Annu Rev Physiol 54, 135-152.

Fucci, D., Crary, M., and Wilson, H. (1976). Vibrotactile and temperature sensory interaction in the human tongue. Percept Mot Skills 43, 263-266.

Gardner, E. P., Martin, J. H., and Jessell, T. M. (2000). The bodily senses. In Principles of Neural Science, Fourth Edition. E. R. Kandel, J. H. Schwartz, and T. M. Jessell, eds. (New York, N.Y.: McGraw-Hill), pp. 430-450.

Gu, G., Caldwell, G. A., and Chalfie, M. (1996). Genetic interactions affecting touch sensitivity in Caenorhabditis elegans. Proc Natl Acad Sci USA 93, 6577-6582.

Harteneck, C., Plant, T. D., and Schultz, G. (2000). From worm to man: three subfamilies of TRP channels. Trends Neurosci 23, 159-166.

Heller, S., Sheane, C. A., Javed, Z., and Hudspeth, A. J. (1998). Molecular markers for cell types of the inner ear and candidate genes for hearing disorders. Proc Natl Acad Sci USA 95, 11400-11405.

Hudspeth, A. J. (1989). How the ear's works work. Nature 341, 397-404.

Hudspeth, A. J., and Gillespie, P. G. (1994). Pulling springs to tune transduction: adaptation by hair cells. Neuron 12, 1-9.

Iredale, P. A. and Hill, S. J. (1993). Increases in intracellular calcium via activation of an endogenous P2-purinoceptor in cultured CHO-K1 cells. Br. J. Pharmacol. 110, 1305-1310.

Johnson, A. K., and Loewy, A. D. (1990). Circumventricular organs and their role in visceral functions. In Central Regulation of Autonomic Function, A. D. Loewy and K. M. Spyer, eds. (Oxford, United Kingdom: Oxford University Press), pp. 247-267.

Kanzaki, M., Nagasawa, M., Kojima, I., Sato, C., Naruse, K., Sokabe, M., and lida, H. (1999). Molecular identification of a eukaryotic, stretch-activated nonselective cation channel. Science 285, 882-886.

Kaplan, J. M., and Horvitz, H. R. (1993). A dual mechanosensory and chemosensory neuron in Caenorhabditis elegans. Proc Natl Acad Sci USA 90, 2227-2231.

Kernan, M., and Zuker, C. (1995). Genetic approaches to mechanosensory transduction. Curr Opin Neurobiol 5, 443-448.

Kernan, M., Cowan, D., and Zuker, C. (1994). Genetic dissection of mechanosensory transduction: mechanoreception-defective mutations of Drosophila. Neuron 12, 1195-1206.

Kyte, J., and Doolittle, R. F. (1982). A simple method for displaying the hydropathic character of a protein. J Mol Biol 157, 105-132.

Lai, C. C., Hong, K., Kinnell, M., Chalfie, M., and Driscoll, M. (1996). Sequence and transmembrane topology of MEC-4, an ion channel subunit required for mechanotransduction in Caenorhabditis elegans. J Cell Biol 133, 1071-1081.

Liedtke, W., Edelman, W., Bieri, P. L., Chiu, F.-C., Cowan, N.J., Kucherlapati, R., and Raine, C. S. (1996) GFAP is necessary for the integrity of CNS white matter architecture and long-term maintenance of myelination. Neuron 17, 607-615.

Lind, R. W., Van Hoesen, G. W., and Johnson, A. K. (1982). An HRP study of the connections of the subformical organ of the rat. J Comp Neurol 210, 265-277.

Lynn, B. (1975). Somatosensory receptors and their CNS connections. Annu Rev Physiol 37, 105-127.

McKinley, M. J., and Oldfield, B. J. (1990). Circumventricular organs. In The Human Nervous System, G. Paxinos, ed. (San Diego, Calif.: Academic Press), pp. 415-438.

McKinley, M. J., Mathai, M. L., Pennington, G., Rundgren, M., and Vivas, L. (1999). Effect of individual or combined ablation of the nuclear groups of the lamina terminalis on water drinking in sheep. Am J Physiol 276, R673-R683.

McKinley, M. J., Bicknell, R. J., Hards, D., McAllen, R. M., Vivas, L., Weisinger, R. S., and Oldfield, B. J. (1992). Efferent neural pathways of the lamina terminalis subserving osmoregulation. Prog Brain Res 91, 395-402.

Merritt, J. E., Armstrong, W. P., Benham, C. D., Hallam, T. J., Jacob, R., Jaxa-Chamiec, A., Leigh, B. K., McCarthy, S. A., Moores, K. E., and Rink, T. J. (1990). SK&F 96365, a novel inhibitor of receptor-mediated calcium entry. Biochem J 271, 515-522.

Meyer, J., Furness, D. N., Zenner, H. P., Hackney, C. M., and Gummer, A. W. (1998). Evidence for opening of hair-cell transducer channels after tip-link loss. J Neurosci 18, 6748-6756.

O'Neill, R. R., Mitchell, L. G., Merril, C. R., and Rasband, W. S. (1989). Use of image analysis to quantitate changes in form of mitochondrial DNA after x-irradiation. Appl Theor Electrophor 1, 163-167.

Penner, R., Neher, E., Takeshima, H., Nishimura, S., and Numa, S. (1989). Functional expression of the calcium release channel from skeletal muscle ryanodine receptor cDNA. FEBS Lett 259, 217-221.

Retief, J. D. (2000). Phylogenetic analysis using PHYLIP. Methods Mol Biol 132, 243-258.

Reuss, H., Mojet, M. H., Chyb, S., and Hardie, R. C. (1997). In vivo analysis of the drosophila light-sensitive channels, TRP and TRPL. Neuron 19, 1249-1259.

Richard, D., and Bourque, C. W. (1995). Synaptic control of rat supraoptic neurones during osmotic stimulation of the organum vasculosum lamina terminalis in vitro. J Physiol (Lond.) 489, 567-577.

Rost, B., and Sander, C. (1993). Prediction of protein secondary structure at better than 70% accuracy. J Mol Biol 232, 584-599.

Rowe, L. B., Nadeau, J. H., Turner, R., Frankel, W. N., Letts, V. A., Eppig, J. T., Ko, M. S., Thurston, S. J., and Birkenmeier, E. H. (1994). Maps from two interspecific backcross DNA panels available as a community genetic mapping resource. Mamm Genome 5, 253-274.

Sackin, H. (1995). Mechanosensitive channels. Annu Rev Physiol 57, 333-353.

Sibbald, J. R., Hubbard, J. I., and Sirett, N. E. (1988). Responses from osmosensitive neurons of the rat subformical organ in vitro. Brain Res 461, 205-214.

Smart, D., Gunthorpe, M. J., Jerman, J. C., Nasir, S., Gray, J., Muir, A. I., Chambers, J. K., Randall, A. D., and Davis, J. B. (2000). The endogenous lipid anandamide is a full agonist at the human vanilloid receptor (hVR1). Br J Pharmacol 129, 227-230.

Sukharev, S. I., Blount, P., Martinac, B., Blattner, F. R., and Kung, C. (1994). A large-conductance mechanosensitive channel in E. coli encoded by mscL alone. Nature 368, 265-268.

Szolcsanyi, I. (2000). Are cannabinoids endogenous ligands for the VR1 capsaicin receptor? Trends Pharmacol Sci 21, 41-42.

Tate, S. S., Yan, N., and Udenfriend, S. (1992). Expression cloning of a Na(+)-independent neutral amino acid transporter from rat kidney. Proc Natl Acad Sci USA 89, 1-5.

Thrasher, T. N., Keil, L. C., and Ramsay, D. J. (1982). Lesions of the organum vasculosum of the lamina terminalis (OVLT) attenuate osmotically-induced drinking and vasopressin secretion in the dog. Endocrinology 110, 1837-1839.

Touchman, J. W., Anikster, Y., Dietrich, N. L., Maduro, V. V., McDowell, G., Shotelersuk, V., Bouffard, G. G., Beckstrom-Sternberg, S. M., Gahl, W. A., and Green, E. D. (2000). The genomic region encompassing the nephropathic cystinosis gene (CTNS): complete sequencing of a 200-kb segment and discovery of a novel gene within the common cystinosis-causing deletion. Genome Res 10, 165-173.

Travis, K. A., and Johnson, A. K. (1993). In vitro sensitivity of median preoptic neurons to angiotensin II, osmotic pressure, and temperature. Am J Physiol 264, 1200-R1205.

Walker, R. G., Willingham, A. T., and Zuker, C. S. (2000). A Drosophila mechanosensory transduction channel. Science 287, 2229-2234.

Weitz, J. (1941). Effect of temperature on sensitivity of the finger. J Exp Psychol 28, 21-36.

Yang, X. C., and Sachs, F. (1989). Block of stretch-activated ion channels in Xenopus oocytes by gadolinium and calcium ions. Science 243, 1068-1071.

Zitt, C., Zobel, A., Obukhov, A. G., Harteneck, C., Kalkbrenner, F., Luckhoff, A., and Schultz, G. (1996) Cloning and functional expression of a human Ca2+-permeable cation channel activated by calcium store depletion. Neuron 16, 1189-1196.

Zygmunt, P. M., Petersson, J., Andersson, D. A., Chuang, H., Sorgard, M., Di Marzo, V., Julius, D., and Hogestatt, E. D. (1999). Vanilloid receptors on sensory nerves mediate the vasodilator action of anandamide. Nature 400, 452-457.

### EXAMPLE 4

### VR-OAC from the Rat Reconstitutes Mechanical and Osmotic Sensitivity in the Caenorhabditis Elegans Mutant OSM-9

Abstract

In order to understand better metazoan animals' response to osmotic and mechanical stimulation, we have expressed the vanilloid receptor related osmotically activated channel (VR-OAC), a candidate vertebrate osmoreceptor, ¹⁻⁴ in the Caenorhabditis elegans mutant osm-9⁵. These worms were complemented for unresponsiveness to hyperosmotic and nose touch stimuli, not for lack of odorant avoidance. Rescue was specific for osm-9 deficits because it respected genetically defined molecular pathways for nose touch and osmotic avoidance. A point mutation in the pore-loop of VR-OAC, methionin to lysin at position 680, markedly reduced complementation, thus indicating that VR-OAC functions as an ion channel in transduction of osmotic and mechanical stimuli in vivo.

All living organisms are endowed with the capability to respond to osmotic and mechanical stimuli⁶⁻⁹. In multicellular organisms with a nervous system, specialized nerve cells transduce the physical stimulus into neuronal excitation¹⁰⁻¹³. The cellular identity of such transducer cells is known in vertebrates, also in insects and other invertebrates. With respect to mechanotransduction in mammals, inner ear hair cells transduce sound and acceleration¹⁴⁻¹⁶, sensory ganglion nerve cells transduce outer and inner surface mechanical stimuli¹³, and, in the brain, circumventricular organ nerve cells transduce systemic osmotic pressure^{9,17}. Whereas the transductory function of these neurosensory cells is known, at least to a degree, the molecular identity of their respective genes is not.

In the invertebrate genetic model organisms, Drosophila melanogaster and Caenorhabditis elegans, mutant lines with particular deficits in response to mechanical and/or osmotic stimuli have been identified through specific screens^{11,18-22}. In C. elegans, genetic evidence from these mutants clearly points towards the existence of two mechanosensory pathways²³⁻²⁶. In the “standard” mechanosensory pathway the stimulus is applied to the worm's torso. Saturation mutagenesis yielded the mec mutants; their respective genes have been cloned. Mec4 and Mec10 encode ion channels, the other Mec genes ancillary genes which most likely form complexes with MEC4 and MEC10²⁷⁻²⁹. In the alternate mechanosensory pathway, responsiveness to nose touch and osmotic avoidance is mediated by the ASH neuron in the worm's head which extends a dendritic process, tipped with a single cilium of characteristic morphology, into the worm nose^{11,26,30}. Sensitivity to nose touch is partly coupled to osmotic avoidance evidenced by the phenotypes of the osm-9, ocr-2 and odr-3 mutants^{5,31,32}. The Osm-9 and Ocr-2 genes encode ion channels and the Odr-3 gene a G_{α}-protein, and all three mutants lack osmotic avoidance and response to nose touch. The phenotypes of the glr-1 and osm-10 mutants indicate, on the other hand, a dissociation of osmotic and mechanical sensing³³⁻³⁵. Glr-1 encodes a postsynaptic C. elegans AMPA-type glutamate receptor and is necessary for sensitivity to nose touch only. Osm-10 encodes a cytoplasmatic protein in the ASH neuron and is necessary for osmotic avoidance only. In D. melanogaster, the recently cloned NompC ion channel is a mechanotransductory ion channel³⁶, NompA a transmembrane protein with a large extracellular matrix domain possibly linking ion channel complexes to the extracellular matrix³⁷.

We and others have recently identified a vertebrate ion channel that is specifically gated by osmotic stimuli, the vanilloid receptor related osmotically activated ion channel (VR-OAC; named also OTRPC4, TRP12 and VRL-2)¹⁻⁴. VR-OAC is a non-selective cation channel that belongs to the osm-9 subfamily of the trp ion channel superfamily^{38,39}. VR-OAC is related to the C. elegans channel OSM-9, which is part of the alternate mechanosensory pathway, and to the mammalian channels vanilloid receptor 1 (VR1) and vanilloid receptor like channel (VRL-1)⁴⁰⁻⁴². The latter two are transductory ion channels for noxious heat^{43,44}. VR-OAC can detect changes in osmotic pressure with great sensitivity^{1,2}. In heterologous expression systems it is gated through hypotonicity within the physiological range. VR-OAC is expressed in neurons of the circumventricular organs where there is no functional blood brain barrier¹. These neurons have been shown to sense systemic osmotic pressure, the most aggressively defended homeostatic setpoint value in vertebrates⁴⁵⁻⁴⁸. Through known projections they relay their findings to magnocellular hypothalamic neurons that secrete antidiuretic hormone into the blood. VR-OAC has also been found to be expressed in other mechanotransductory cells, namely inner ear hair cells, trigeminal ganglion intermediate-size neurons, and Merkel cells in close proximity to whiskers¹. In mechanosensory transducer cells, VR-OAC could possibly function in maintaining cellautonomous osmotic and ionic homeostasis. As an alternative, VR-OAC could form a mechanotransductory ion channel, perhaps complexed with other channel components forming heteromultimers. The C. elegans osm-9 mutant is characterized by lack of response to nose touch, lack of avoidance of hyperosmotic stimuli and lack of avoidance of specific volatile odorant repellents, e.g. octanone. We have addressed the question whether VR-OAC is a vertebrate functional orthologue of the C. elegans channel OSM-9 by expressing rat VR-OAC in osm-9 worms.

The coding region of the rat VR-OAC cDNA was cloned into the C. elegans expression vector pPD49.26 that contained the sra6 promotor. This promotor directs expression in the ASH and ASI neurons. Purified DNA was injected into the gonad of hermaphroditic worms, strain N2, together with the marker gene elt-2/green fluorescent protein (GFP) which directs expression of GFP in C. elegans intestinal cells. Transgenic offspring could be identified by GFP fluorescence, and VR-OAC transgenic lines of worms were generated. Likewise, several transgenic lines of worms were established and maintained on an osm-9 (ky-10 allele) genetic background. In these worms, an osmotic avoidance in response to hyperosmotic stimuli was clearly present, as was sensitivity to nose touch (FIGS. 15A and 15B). The avoidance reactions were assayed on single animals as previously described³⁴. Odorant avoidance in response to octanone was not present. This indicates a partial rescue for lack of osmotic avoidance and for lack of response to nose touch, and no rescue for lack of odorant avoidance. In order to confirm that the observed rescue is mediated by the ASH neuron, both ASH neurons were laser ablated in osm-9/VR-OAC transgenic (tg) worms. This procedure completely abolished rescue (FIGS. 15A and 15B). In order to visualize subcellular localization of the VR-OACtg in the ASH neuron, GFP was tagged to the C-terminus of VR-OAC. The tagged protein could be localized to the cilia of the ASH neuron in the worm nose where fluorescence was stronger than in any other part of this neuron. Rescue was not impaired in VR-OAC/GFPtg osm-9 worms (FIGS. 15A and 15B).

In order to understand better the specificity of the observed rescue, VR-OACtg worms were crossed to C. elegans mutants which genetically define the alternate mechanosensory pathway. VR-OACtg worms of the following mutant lines were generated: ocr-2, odr-3, glr-1, osm-10 and double-mutants of the former with osm-9. These lines were tested for osmotic avoidance, nose touch and odorant avoidance. The results of these control experiments indicated that transgenic expression of VR-OAC leads to specific complementation of the osm-9 deficits only (FIGS. 16A and 16B). In another control experiment addressing the specificity of the observed rescue, we expressed rat VR1 under the control of the sra6 promotor in ASH neurons of wild type and osm-9 worms. No rescue was observed. However, in osm-9 as well as in wild type worms tg for VR1 there was a strong avoidance response to vanilloids which was absent in non-tg worms.

Next, we wanted to use the transgenic rescue of osm-9 by VR-OAC as an in-vivo model in order to better understand structure-function relationships of VR-OAC. Gross deletions were constructed lacking either the N-terminal intracellular domain (aminoacid [AA] 1-410 of VR-OAC), the C-terminal intracellular domain (AA 741-781 of VR-OAC) or both. Three lines of worms were established for each of the gross deletions. The line with the most pronounced rescue as an indication of highest expression level of tg was investigated more in depth. The data indicated that VROAC without both its N-terminus and C-terminus still rescues lack of osmotic avoidance and lack of response to nose touch, albeit at a lower level than the intact channel (FIGS. 17A and 17B). The N-terminus and the C-terminus thus appear dispensable for the response to osmotic and mechanical stimuli, and, most likely, for proper intracellular transport in the ASH neuron. In addition to these gross deletions, we wanted to learn more about the structure-function relationship of strategically positioned single amino acid exchanges in the pore-loop domain of VR-OAC. As for the gross deletions, three lines of worms were generated for each point mutation. Whereas an exchange in charge at position 671 and 682 (D671K, D682K) did not alter rescue substantially, introduction of a positive charge at position 680 (M680K) did strikingly reduce rescue (FIGS. 17A and 17B). However, this did not reach the level of uncomplemented osm-9. These findings and previous work on other ion channels ^{49,50} point towards AA680 of VR-OAC and similarly positioned AA residues of family members of the osm-9 ion channel subfamily as a key molecular determinant of pore-loop and thus ion channel functioning. Moreover, our data clearly indicate that VR-OAC functions as an ion channel in-vivo. It strongly argues against a hypothetical scenario where VR-OAC solely functions as a “chaperone” for OCR-2 to reach its appropriate location in the ASH cilium. Whereas a residual rescue could be observed in osm-9/VR-OAC[M680K]tg worms, VR-OAC[M680K] totally lacked function in tissue culture cells. This finding makes it likely that VR-OAC participates in the formation of heteromultimeric complexes together with other ion channels. Assuming that binding partners probably originate from the same subfamily of ion channels, the only possible ion channel binding partner for VR-OAC in osm-9 worms is OCR-2. OCR-2 is the sole other member of the osm-9 subfamily of trp ion channels that is expressed in the ASH neuron, provided there are no additional family members in yetunknown portions of the C. elegans genome. Why did VR-OAC respond to hypotonic osmotic stimuli in heterologous tissue culture expression systems, but complemented lack of osmotic avoidance of hypertonic osmotic stimuli in osm-9 worms ? We co-expressed VR-OAC with OCR-2 and, as an alternate strategy in order to co-express two mammalian genes, with VRsv⁵¹ in three different lines of commonly used tissue culture cells. In none of these experiments could we observe calcium influx in response to hypertonic stimuli (data not shown). In addition, we subjected C. elegans w.t. and osm-9 to hypotonic osmotic stimuli which elicited an avoidance response in both (data not shown). We hypothesize binding partners to VROAC that make the channel respond to the opposite stimulus it responds to in heterologous tissue culture expression systems. Proteins with an extracellular matrix domain that acts as lever and/or “push-pull converter” can be hypothesized ^{37,52}.

Transgenic expression of rat VR-OAC in the C. elegans mutant osm-9 reveals the presence of true functional molecular orthology of VR-OAC to the C. elegans ion channel OSM-9 with respect to sensing osmotic and mechanical stimuli. Rescue of a genetically deficient transductory function by a gene that has evolved over more than 100 million years of molecular evolution is unprecedented. In addition, these results implicate VR-OAC not only to participate in sensing osmotic stimuli in a live organism, but also in mechanotransduction. Rescue is remarkable in view of 30% amino acid identity of the two channels. In an alignment of VR-OAC and OSM-9, identical or conservatively exchanged amino acids are located mainly in the transmembrane domain. In view of this, the fact that both the N-terminal and the Cterminal intracellular domain of VR-OAC are dispensable for rescue becomes less surprising. With respect to the profile of the rescue where sensitivity to nose touch and osmotic avoidance can be reconstituted independent of the N- and C-terminus of VROAC, and where not even intact VR-OAC rescues lack of odorant avoidance, it is attractive to speculate along the following two lines. Either the odorant repellents bind to G-protein coupled receptors and VR-OAC cannot function in downstream signalling instead of OSM-9, possibly because the C- and N-termini are too different. Alternatively, odorant repellents bind to OSM-9 directly and VR-OAC cannot back-up for this function of OSM-9, possibly because the ligand binding sites are intracellular N- and C-terminal domains, which are too different between species. Future studies will address rescue of osm-9 by transgenic expression of OSM-9 and variants of it and by chimeric molecules between OSM-9 and VR-OAC.

With respect to the association/dissociation of osmo- and mechano-sensation in the alternate mechanosensory pathway in C. elegans, our results indicate the following. First, through the specific profile of the rescue it is clear that there are two different signalling pathways that do not function without OSM-9, namely signalling in response to odorant repellents on the one hand and response to hyperosmotic and nose touch stimuli on the other hand. Other mutants which define the alternate mechanosensory pathway imply an association of mechano-osmo-transduction. As for Osm-9, Ocr-2 encodes an ion channel. If mechano- and osmo-transduction are indeed linked and the transduction channel consists of members of the osm-9 subfamily, then this can only be through complexing of OSM-9 with OCR-2 since no other members of this family are expressed in the ASH neuron. In addition, the rescue of osm-9 by ΔN-VR-OAC-ΔC implies that key binding sites do not reside in the N- and C-terminal domains. However, such a working hypothesis has to be reconciled with the phenotypes of the glr-1 and the osm-10 mutants³³⁻³⁵. The postsynaptic C. elegans glutamate receptor GLR-1 is necessary for nose touch only, and the intracellular protein OSM-10 of the ASH neuron is necessary for osmotic avoidance only. One possibility consists of heteromultimeric ion channel complexes that have the same ionophore core, OSM-9 and OCR-2, and a different set of intracellular and extracellular binding partners that define the specificity of the response. In the standard mechanosensory pathway in C. elegans, extracellular matrix proteins are considered likely binding partners to heteromultimeric ion channels. This points towards a specific role for anchoring in the extracellular matrix of mechanoreceptor complexes. VR-OAC's function in a heterologous expression system was significantly altered, though not abrogated, by deletion of the N-terminus containing the ankyrin repeat domains¹. This points toward intracellular anchoring as having an important role in osmosensory heteromultimeric complexes. OSM-10 is a protein that could play exclusively a role in the formation of specifically osmosensory complexes. With respect to the specific mechanosensory defect of glr-1, it is possible that mechanosensory specificity is achieved through the amount of calcium influx into the ASH neuron¹¹. A minor amount of calcium influx, perhaps in a localized, subcellular fashion, in response to a mechanical stimulus could lead to glutamatergic transmitter release only, whereas an osmotic stimulus would lead to a higher amount of calcium influx which would in turn release glutamatergic and other transmitters so that there is no obvious lack of osmotic avoidance in the glr-1 mutant. To obtain clarity, future studies will involve imaging studies of gene expression of other osmoreceptor/mechanoreceptor candidate ion channels in C. elegans, physiological recordings from ASH neurons in live worms in response to osmotic and mechanical stimuli, and recordings from yet-to-be-established heterologous expression systems where an umambiguous response latency upon mechanical stimulation can be measured.

In sum, we have demonstrated the specific rescue of lack of osmotic avoidance and lack of response to nose touch in the C. elegans mutant osm-9 by transgenic expression of rat VR-OAC in the ASH neuron. Lack of odorant avoidance in these worms was not complemented, and an amino acid exchange M680K in the pore-loop domain of VR-OAC drastically reduced rescue. These results indicate that VR-OAC functions as an ion channel in the transduction of osmotic and mechanical stimuli in vivo, and that VR-OAC and OSM-9 are true functional orthologues thus bridging more than 100 million years of molecular evolution. Our results underscore the general relevance of the invertebrate genetic model organism C. elegans in the molecular understanding of mechanotransduction. BNC1 is a mammalian orthologue of the MEC-4/MEC-10 mechanotransductory ion channel identified in touch-insensitivity screens in C. elegans more than a decade ago^{53,54}. In this respect, we did not fail to notice that mice lacking the BNC1 sodium channel have subtle, yet specific mechanosensory defects⁵⁵, and that additional evidence points toward the BNC1 channel being a mechanoreceptor⁵⁶.

Materials and Methods

The generation of transgenic C. elegans and assessments for osmostic avoidance, nose touch and odorant avoidance were done in accordance with the methods reported by Bargmann (Bargmann, C. I. & Kaplan, J. M. Annu Rev Neurosci 21, 279-308 (1998); Bargmann, C. I., Thomas, J. H. & Horvitz, H. R. Cold Spring Harb Symp Quant Biol 55, 529-38 (1990) and Hart (Hart, A. C., Sims, S. & Kaplan, J. M. Nature 378, 82-5 (1995); Hart, A. C., Kass, J., Shapiro, J. E. & Kaplan, J. M. J Neurosci 19, 1952-8 (1999).

### REFERENCES

1. Liedtke, W. et al. Vanilloid receptor-related osmotically activated channel (VR-OAC), a candidate vertebrate osmoreceptor. Cell 103, 525-35 (2000).

2. Strotmann, R., Harteneck, C., Nunnenmacher, K., Schultz, G. & Plant, T. D. OTRPC4, a nonselective cation channel that confers sensitivity to extracellular osmolarity. Nat Cell Biol 2, 695-702. (2000).

3. Wissenbach, U., Bodding, M., Freichel, M. & Flockerzi, V. Trpl2, a novel Trp related protein from kidney. FEBS Lett 485, 127-34. (2000).

4. Delany, N. S. et al. Identification and characterization of a novel human vanilloid receptor-like protein, VRL-2. Physiol Genomics 4, 165-74. 2001).

5. Colbert, H. A., Smith, T. L. & Bargmann, C. I. OSM-9, a novel protein with structural similarity to channels, is required for olfaction, mechanosensation, and olfactory adaptation in Caenorhabditis elegans. J Neurosci 17, 8259-69 (1997).

6. Sackin, H. Mechanosensitive channels. Annu Rev Physiol 57, 333-53 (1995).

7. Garcia-Anoveros, J. & Corey, D. P. The molecules of mechanosensation. Annu Rev Neurosci 20, 567-94 (1997).

8. Hamill, 0. P. & Martinac, B. Molecular basis of mechanotransduction in living cells. Physiol Rev 81, 685-740. (2001).

9. Bourque, C. W. & Oliet, S. H. Osmoreceptors in the central nervous system. Annu Rev Physiol 59, 601-19 (1997).

10. Detweiler, P. B. in Textbook of Physiology: Excitable Cells and Neurophysiology (eds. Patton, H. D., Fuchs, A. F., Hille, B., Sher, A. M. & Streiner, R.) 98-129 (Saunders, Philadelphia, Pa., 1989).

11. Bargmann, C. I. & Kaplan, J. M. Signal transduction in the Caenorhabditis elegans nervous system. Annu Rev Neurosci 21, 279-308 (1998).

12. Hudspeth, A. J. & Logothetis, N. K. Sensory systems. Curr Opin Neurobiol 10, 631-41. (2000).

13. Gardner, E. P., Martin, J. H. & Jessell, T. M. in Principles of Neural Science (ed. Kandel E R, S. J., Jessell T M) 430-450 (2000).

14. Hudspeth, A. J. How the ear's works work. Nature 341, 397-404 (1989).

15. Hudspeth, A. J. & Gillespie, P. G. Pulling springs to tune transduction: adaptation by hair cells. Neuron 12, 1-9 (1994).

16. Gillespie, P. G. Molecular machinery of auditory and vestibular transduction. Curr Opin Neurobiol 5, 449-55 (1995).

17. Richard, D. & Bourque, C. W. Synaptic control of rat supraoptic neurones during osmotic stimulation of the organum vasculosum lamina terminalis in vitro. J Physiol (Lond) 489, 567-77 (1995).

18. Kernan, M., Cowan, D. & Zuker, C. Genetic dissection of mechanosensory transduction: mechanoreception-defective mutations of Drosophila. Neuron 12, 1195-206 (1994).

19. Kernan, M. & Zuker, C. Genetic approaches to mechanosensory transduction. Curr Opin Neurobiol 5, 443-8 (1995).

20. Chalfie, M. & Sulston, J. Developmental genetics of the mechanosensory neurons of Caenorhabditis elegans. Dev Biol 82, 358-70. (1981).

21. Chalfie, M. et al. The neural circuit for touch sensitivity in Caenorhabditis elegans. J Neurosci 5, 956-64. (1985).

22. Duggan, A., Garcia-Anoveros, J. & Corey, D. P. Insect mechanoreception: what a long, strange TRP it's been. Curr Biol 10, R384-7. (2000).

23. Chalfie, M. Touch receptor development and function in Caenorhabditis elegans. J Neurobiol 24, 1433-41. (1993).

24. Chalfie, M. Homeobox genes in Caenorhabditis elegans. Curr Opin Genet Dev 3, 275-7. (1993).

25. Kaplan, J. M. & Horvitz, H. R. A dual mechanosensory and chemosensory neuron in Caenorhabditis elegans. Proc Natl Acad Sci U S A 90, 2227-31 (1993).

26. Bargmann, C. I., Thomas, J. H. & Horvitz, H. R. Chemosensory cell function in the behavior and development of Caenorhabditis elegans. Cold Spring Harb Symp Quant Biol 55, 529-38 (1990).

27. Tavernarakis, N. & Driscoll, M. Molecular modeling of mechanotransduction in the nematode Caenorhabditis elegans. Annu Rev Physiol 59, 659-89 (1997).

28. Huang, M. & Chalfie, M. Gene interactions affecting mechanosensory transduction in Caenorhabditis elegans. Nature 367, 467-70. (1994).

29. Garcia-Anoveros, J., Ma, C. & Chalfie, M. Regulation of Caenorhabditis elegans degenerin proteins by a putative extracellular domain. Curr Biol 5, 441-8. (1995).

30. Thurm, U. et al. Cilia specialized for mechanoreception. J Submicrosc Cytol 15, 151-155 (1983).

31. Roayaie, K., Crump, J. G., Sagasti, A. & Bargmann, C. I. The G alpha protein ODR-3 mediates olfactory and nociceptive function and controls cilium morphogenesis in C. elegans olfactory neurons. Neuron 20, 55-67 (1998).

32. Tobin, D. & Bargmann, C. I. ocr-2 encodes a putative C. elegans ion channel involved in mechanosensation, osmosensation and chemical sensing. Neuron (2001).

33. Hart, A. C., Sims, S. & Kaplan, J. M. Synaptic code for sensory modalities revealed by C. elegans GLR-1 glutamate receptor. Nature 378, 82-5 (1995).

34. Hart, A. C., Kass, J., Shapiro, J. E. & Kaplan, J. M. Distinct signaling pathways mediate touch and osmosensory responses in a polymodal sensory neuron. J Neurosci 19, 1952-8 (1999).

35. Maricq, A. V., Peckol, E., Driscoll, M. & Bargmann, C. I. Mechanosensory signalling in C. elegans mediated by the GLR-1 glutamate receptor [published erratum appears in Nature 1996 February 22;379(6567):749]. Nature 378, 78-81 (1995).

36. Walker, R. G., Willingham, A. T. & Zuker, C. S. A Drosophila mechanosensory transduction channel. Science 287, 2229-34 (2000).

37. Chung, Y. D., Zhu, J., Han, Y. & Kernan, M. J. nompA encodes a PNSspecific, ZP domain protein required to connect mechanosensory dendrites to sensory structures. Neuron 29, 415-28. (2001).

38. Harteneck, C., Plant, T. D. & Schultz, G. From worm to man: three subfamilies of TRP channels. Trends Neurosci 23, 159-166 (2000).

39. Clapham, D. E., Runnels, L. W. & Strubing, C. The TRP ion channel family. Nat Rev Neurosci 2, 387-96. (2001).

40. Caterina, M. J. et al. The capsaicin receptor: a heat-activated ion channel in the pain pathway [see comments]. Nature 389, 816-24 (1997).

41. Caterina, M. J., Rosen, T. A., Tominaga, M., Brake, A. J. & Julius, D. A capsaicin-receptor homologue with a high threshold for noxious heat. Nature 398, 436-41 (1999).

42. Caterina, M. J. et al. Impaired Nociception and Pain Sensation in Mice Lacking the Capsaicin Receptor. Science 288, 306-313 (2000).

43. Caterina, M. J. & Julius, D. Sense and specificity: a molecular identity for nociceptors. Curr Opin Neurobiol 9, 525-30 (1999).

44. Caterina, M. J. & Julius, D. The vanilloid receptor: a molecular gateway to the pain pathway. Annu Rev Neurosci 24, 487-517 (2001).

45. Denton, D. A., McKinley, M. J. & Weisinger, R. S. Hypothalamic integration of body fluid regulation. Proc Natl Acad Sci U S A 93, 7397-404 (1996).

46. Lind, R. W., Van Hoesen, G. W. & Johnson, A. K. An HRP study of the connections of the subformical organ of the rat. J Comp Neurol 210, 265-77 (1982).

47. McKinley, M. J. et al. Efferent neural pathways of the lamina terminalis subserving osmoregulation. Prog Brain Res 91, 395-402 (1992).

48. McKinley, M. J., Mathai, M. L., Pennington, G., Rundgren, M. & Vivas, L. Effect of individual or combined ablation of the nuclear groups of the lamina terminalis on water drinking in sheep. Am J Physiol 276, R673-83 (1999).

49. Nilius, B. et al. The Single Pore Residue Asp542 Determines Ca2 +Permeation and Mg2+ Block of the Epithelial Ca2+ Channel. J Biol Chem 276, 1020-1025 (2001).

50. Garcia-Martinez, C., Morenilla-Palao, C., Planells-Cases, R., Merino, J. M. & Ferrer-Montiel, A. Identification of an aspartic residue in the P-loop of the vanilloid receptor that modulates pore properties. J Biol Chem 275, 32552-8. (2000).

51. Schumacher, M. A., Moff, I., Sudanagunta, S. P. & Levine, J. D. Molecular cloning of an N-terminal splice variant of the capsaicin receptor. Loss of N-terminal domain suggests functional divergence among capsaicin receptor subtypes. J Biol Chem 275, 2756-62 (2000).

52. Du, H., Gu, G., William, C. M. & Chalfie, M. Extracellular proteins needed for C. elegans mechanosensation. Neuron 16, 183-94. (1996).

53. Price, M. P., Snyder, P. M. & Welsh, M. J. Cloning and expression of a novel human brain Na⁺ channel. J Biol Chem 271, 7879-82. (1996).

54. Garcia-Anoveros, J., Derfler, B., Neville-Golden, J., Hyman, B. T. & Corey, D. P. BNaC1 and BNaC2 constitute a new family of human neuronal sodium channels related to degenerins and epithelial sodium channels. Proc Natl Acad Sci U S A 94, 1459-64. (1997).

55. Price, M. P. et al. The mammalian sodium channel BNC1 is required for normal touch sensation. Nature 407, 1007-11. (2000).

56. Garcia-Anoveros, J., Samad, T. A., Woolf, C. J. & Corey, D. P. Transport and localization of the DEG/ENaC ion channel BNaClalpha to peripheral mechanosensory terminals of dorsal root ganglia neurons. J Neurosci 21, 2678-86. (2001).

This invention may be embodied in other forms or carried out in other ways without departing from the spirit or essential characteristics thereof. The present disclosure is therefore to be considered as in all respects illustrative and not restrictive, the scope of the invention being indicated by the appended claims, and all changes which come within the meaning and range of equivalency are intended to be embraced therein.

Various references are cited throughout this specification, each of which is incorporated herein by reference in its entirety.

We claim:

1. A method for modulating mechanoreception or mechanosensation in a mammal comprising administering to said mammal an effective amount of VR-OAC polypeptide, or active fragments or portions thereof.

2. The method of claim 1 wherein said VR-OAC polypeptide comprises the sequence set out in any of SEQ ID NOS: 2, 4, 8 or 9.

3. The method of claim 1 wherein said active fragment or portion of VR-OAC polypeptide comprises the sequence set not in any of SEQ ID NOS: 5, 6, or 7.

4. A method for treating a condition characterized by altered mechanoreception or mechanosensation in a mammal comprising administering to said mammal an effective amount of VR-OAC polypeptide, or active fragments or portions thereof, wherein said VR-OAC polypeptide comprises the sequence set out in any of SEQ ID NOS: 2, 4, 8 or 9.

5. The method of claim 4, wherein said active fragment or portion of VR-OAC polypeptide comprises the sequence set out in any of SEQ ID NOS: 5, 6 or 7.

6. The method of claim 4, wherein said condition characterized by altered mechanoreception or mechanosensation is selected from hearing disorders, vertigo of labyrinthine origin including motion sickness, Meniere disease, neurological disorders (including ataxia due to alterations of afferent input to the CNS, and paraesthesia), male infertility, immune dysfunction with alterations of antigen presentation (including HIV infection), obesity and diabetes mellitus, chronic obstructive lung disorder, bronchial asthma, sexual dysfunction due to sensory deficits, blindness due to corneal or retinal causes, and skin disorders (including psoriasis, pemphigus vulgaris and other forms of pemphigoids, pruritus, allergic skin diseases).

7. A method for modulating mechanoreception or mechanosensation in a mammal comprising introducing to said mammal a nucleic acid vector capable of expressing an effective amount of VR-OAC polypeptide, or active fragments or portions thereof, wherein said VR-OAC polypeptide comprises the amino sequence set out in any of SEQ ID NOS: 2, 4, 8 or 9.

8. The method of claim 7 wherein said active fragment or portion of VR-OAC polypeptide comprises the sequence set not in any of SEQ ID NOS: 5, 6, or 7.

9. A method for determining whether a subject is suffering from altered mechanoreception or mechanosensation comprising determining the expression of VR-OAC polypeptide or ribonucleic acid capable of encoding VR-OAC polypeptide.

10. The method of claim 9 comprising the steps of:
a) contacting a sample from a subject for which altered mechanoreception or mechanosensation is suspected with an antibody to the VROAC polypeptide under conditions that allow binding of the VR-OAC polypeptide to the antibody to occur; and
b) detecting whether binding has occurred between the VR-OAC from the sample and the antibody;
wherein the detection of binding indicates that presence or activity of the VR-OAC polypeptide in the sample.

11. A method of screening for modulators of mechanoreception or mechanosensation comprising the steps of:
a) contacting a sample in the presence of a candidate modulator with an antibody to the VR-OAC polypeptide under conditions that allow binding of the VR-OAC polypeptide to the antibody to occur; and
b) detecting whether binding has occurred between the VR-OAC from the sample and the antibody;
wherein the detection of binding indicates that presence or activity of the VR-OAC polypeptide in the sample.

12. A method of screening for modulators of mechanoreception or mechanosensation comprising the steps of:
a) contacting a C. elegans osm-9 mutant which expresses VR-OAC polypeptide with a candidate modulator; and
b) assessing the activity of VR-OAC in the presence of said modulator by determining nose touch sensitivity and/or osmotic avoidance in said C. elegans mutant.

13. A biosensor or nanotechnological device, which comprises as one of its components the VR-OAC polypeptide or active fragments or portions thereof.

14. The biosensor or technological device of claim 13 wherein said VR-OAC polypeptide or active fragments or portions thereof comprises the amino acid sequence set out in any of SEQ ID NOS: 2, 4, 5, 6, 7, 8 or 9.
